(19) United States
(12) Patent Application Publication
(10) Pub. No.: US 2009/0209598 A1
(43) Pub. Date: Aug. 20, 2009

(54) Resorcine Derivatives and Their Use as Pesticides

(76) Inventors: Michael Puhl, Lampertheim (DE); Markus Kordes, Frankenthal (DE); Matthias Pohlman, Heidelberg (DE); Norbert Götz, Worms (DE); Michael Rack, Eppelheim (DE); Jürgen Langewald, Mannheim (DE); Felix Schneider, Hassloch (DE); Douglas D. Anspaugh, Apex, NC (US); Deborah L. Culbertson, Fuquay Varina, NC (US); Hassan Oloumi-Sadeghi, Raleigh, NC (US); David G. Kuhn, Apex, NC (US)

Correspondence Address: BRINKS, HOFER, GILSON & LIONE, P.O. BOX 1340, MORRISVILLE, NC, 27560, US

(21) Appl. No.: 12/093,543

(22) PCT Filed: Nov. 10, 2006
(86) PCT No.: PCT/EP06/68329

§ 371 Date: Sep. 4, 2008

Related U.S. Application Data
(60) Provisional application No. 60/736,254, filed on Nov. 14, 2005.

(51) Int. Cl. A01N 43/40; A01N 43/38; C07D 401/04; C07D 209/54

(52) U.S. Cl. 514/341; 514/409; 546/275.4; 548/408

(57) ABSTRACT

This invention relates to resorcine derivatives of formula (I) wherein R¹ is phenyl or a 5- to 6-membered heteroaromatic ring which may contain 1 to 3 heteroatoms selected from oxygen, nitrogen and sulfur, wherein phenyl or the heteroaromatic ring may be fused to a ring selected from phenyl and a 5- to 6-membered saturated, partially unsaturated or aromatic heterocyclic ring which may contain 1 to 3 heteroatoms selected from oxygen, nitrogen and sulfur, wherein phenyl or the 5- to 6-membered heteroaromatic ring or the respective fused ring systems may be unsubstituted or substituted by any combination of 1 to 6 optionally substituted groups R³, wherein R³ is halogen, cyano, nitro, hydroxy, mercapto, amino, alkyl, alkenyl, alkynyl, cycloalkyl, cycloalkenyl, haloalkyl, haloalkenyl, haloalkynyl, halocycloalkyl, halocycloalkenyl, alkoxy, alkenyloxy, alkynyloxy, haloalkoxy, haloalkenyloxy, haloalkynyloxy, alkylthio, alkenylthio, alkynylthio, haloalkylthio, haloalkenylthio, haloalkynylthio, alkylamino, alkenylamino, alkynylamino, dialkylamino, dialkenylamino, dialkynylamino, alkyl-alkenylamino, alkyl-alkynylamino, alkenyl-alkynylamino, trialkylsilyl, or phenyl or a 5- to 7-membered saturated or partially unsaturated heterocyclic ring which may contain 1 to 3 heteroatoms selected from oxygen, sulfur and nitrogen or a 5- to 6-membered heteraromatic ring system which may contain 1 to 4 heteroatoms selected from oxygen, nitrogen and sulfur, which phenyl and which heteroaromatic ring may be bonded via an oxygen or a sulfur atom, or —C(=G)Ra, —C(=G)ORa, —C(=G)NRa2, —C(=G)[N═SRa2], —C(═NORa)Ra, —C(═NORa)NRa2, —C(═NNRa2)Ra, —OC(=G)-OC(=G)ORa, N═SRa2, —NRaC(=G)Ra, —N[C(=G)Ra]2, —NRaC(=G)ORa, —C(=G)NRa—NRa2, —C(=G)NRa—NRa[C(=G)Ra], —NRa—C(=G)NRa2, —NRa—NRaC(=G)Ra, —NRa—N[C(=G)Ra]2, —N[(C=G)Ra]—NRa2, —NRa—NRa[(C=G)GRa], —NRa[(C=G)NRa2, —NRa[C═NRa]Ra, —NRa(C═NRa)NRa2, —O—NRa2, —O—NRa(C=G)Ra, —SO2NRa2, —NRaSO2Ra, —S(═O)Ra, —S(═O)2Ra, —SO2ORa, or —OSO2Ra, wherein G is oxygen or sulfur and Ra is as defined in the description, R² is hydrogen, halogen, cyano, alkyl, haloalkyl, alkenyl, haloalkenyl, alkynyl, haloalkynyl, alkoxy, or haloalkoxy, x is 2, 3, 4, 5, 6, or 7, or the diastereomers, enantiomers, salts or N-oxides thereof, with the proviso that R¹ is not 5-chloro-2,2,-dimethyl-2,3-dihydrobenzo[b]furan-7-yl when R² is hydrogen and x is 3 or 4, processes and intermediates for the preparation of compounds I, use of compounds I for combating insects, acarids, or nematodes, and a method for treating, controlling, preventing or protecting animals against infestation or infection by parasites using compounds I.

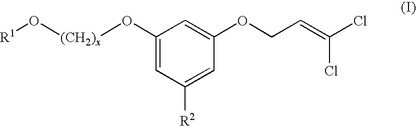

The present invention relates to resorcine derivatives of formula I

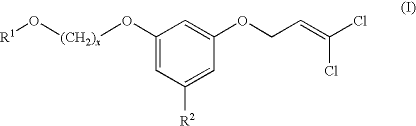

wherein
R¹ is phenyl or a 5- to 6-membered heteroaromatic ring which may contain 1 to 3 heteroatoms selected from oxygen, nitrogen and sulfur, wherein phenyl or the heteroaromatic ring may be fused to a ring selected from phenyl and a 5- to 6-membered saturated, partially unsaturated or aromatic heterocyclic ring which may contain 1 to 3 heteroatoms selected from oxygen, nitrogen and sulfur,
   wherein phenyl or the 5- to 6-membered heteroaromatic ring or the respective fused ring systems may be unsubstituted or substituted by any combination of 1 to 6 groups R³;
   R³ is halogen, cyano, nitro, hydroxy, mercapto, amino, C₁-C₆-alkyl, C₂-C₆-alkenyl, C₂-C₆-alkynyl, C₃-C₆-cycloalkyl, C₃-C₆-cycloalkenyl, C₁-C₆-haloalkyl, C₂-C₆-haloalkenyl, C₂-C₆-haloalkynyl, C₃-C₆-halocycloalkyl, C₃-C₆-halocycloalkenyl, C₁-C₆-alkoxy, C₂-C₆-alkenyloxy, C₃-C₆-alkynyloxy, C₁-C₆-haloalkoxy, C₂-C₆-haloalkenyloxy, C₃-C₆-haloalkynyloxy, C₁-C₆-alkylthio, C₂-C₆-alkenylthio, C₃-C₆-alkynylthio, C₁-C₆-haloalkylthio, C₂-C₆-haloalkenylthio, C₃-C₆-haloalkynylthio C₁-C₆-alkylamino, C₂-C₆-alkenylamino, C₂-C₆-alkynylamino,
      di(C₁-C₆-alkyl)amino, di(C₂-C₆-alkenyl)amino, di(C₂-C₆-alkynyl)amino, C₁-C₆-alkyl-C₂-C₆-alkenylamino, C₁-C₆-alkyl-C₂-C₆-alkynylamino, C₁-C₆-alkenyl-C₂-C₆-alkynylamino, tri(C₁-C₁₀)alkylsilyl, or
      phenyl or a 5- to 7-membered saturated or partially unsaturated heterocyclic ring which may contain 1 to 3 heteroatoms selected from oxygen, sulfur and nitrogen or a 5- to 6-membered heteraromatic ring system which may contain 1 to 4 heteroatoms selected from oxygen, nitrogen and sulfur, which phenyl and which heteroaromatic ring may be bonded via an oxygen or a sulfur atom,
      wherein the above groups R³ are unsubstituted, or the hydrogen atoms in these groups may all or in part be replaced with any combination of groups selected from R^{a}, or
      —C(=G)R^{a}, —C(=G)OR^{a}, —C(=G)NR^{a}₂, —C(=G)[N═SR^{a}₂], —C(═NOR^{a})R^{a}, —C(═NOR^{a})NR^{a}₂, —C(═NNR^{a}₂)R^{a}, —OC(=G)-OC(=G)OR^{a}, N═SR^{a}₂, —NR^{a}C(=G)R^{a}, —N[C(=G)R^{a}]2, —NR^{a}C(=G)OR^{a}, —C(=G)NR^{a}—NR^{a}₂, —C(=G)NR^{a}—NR^{a}[C(=G)R^{a}], —NR^{a}—C(=G)NR^{a}₂, —NR^{a}—NR^{a}C(=G)R^{a}, —NR^{a}—N[C(=G)R^{a}]₂, —N[(C=G)R^{a}]—NR^{a}₂, —NR^{a}—NR^{a}[(C=G)GR^{a}], —NR^{a}[(C=G)NR^{a}₂, —NR^{a}[C═NR^{a}]R^{a}, —NR^{a}(C═NR^{a})NR^{a}₂, —O—NR^{a}₂, —O—NR^{a}(C=G)R^{a}, —SO₂NR^{a}₂, —NR^{a}SO₂R^{a}, —S(═O)R^{a}, —S(═O)₂R^{a}, SO₂OR^{a}, or —OSO₂R^{a};
         G is oxygen or sulfur;
         R^{a} is each independently halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₈-halocycloalkenyl, ORⁱ, SRⁱ, S(═O)Rⁱ, S(═O)₂Rⁱ, NRⁱR^{j}, —S(═O)₂NRⁱR, C(═O)Rⁱ, C(═O)ORⁱ, C(═O)NRⁱR^{j}, C(═NORⁱ)R^{j}, —NRⁱC(=G)R^{j}, —N[C(=G)Rⁱ]2, —NRⁱC(=G)OR^{j}, —C(=G)NRⁱ—NR^{j}₂, —SO2NRⁱR^{j}, —NRⁱSO₂R^{j}, SiRⁱ_{y}R^{j}_{3-y}(y is 0 to 3), or
            phenyl or a 5- to 6-membered heteraromatic ring which may contain 1 to 4 heteroatoms selected from oxygen, nitrogen and sulfur, wherein the carbon atoms in phenyl or in the heteroaromatic ring may be substituted with 1 to 5 halogens;
            Rⁱ, R^{j} are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₆-halocycloalkenyl, C₁-C₆-alkoxy, C₂-C₆-alkenyloxy, C₂-C₆-alkynyloxy, C₁-C₆-haloalkoxy, C₂-C₆-haloalkenyloxy;
R² is hydrogen, halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, or C₁-C₆-haloalkoxy;
x is 2, 3, 4, 5, 6, or 7;
   or the diastereomers, enantiomers, salts or N-oxides thereof, with the proviso that R¹ is not 5-chloro-2,2,-dimethyl-2,3-dihydrobenzo[b]furan-7-yl when R² is hydrogen and x is 3 or 4.

In addition, the present invention relates to processes for preparing the compounds I, pesticidal compositions comprising compounds I and methods for the control of insects, acarids or nematodes by contacting the insect, acarid or nematode or their food supply, habitat or breeding grounds with a pesticidally effective amount of compounds or compositions of formula I.

Moreover, the present invention also relates to a method of protecting growing plants from attack or infestation by insects or acarids by applying to the foliage of the plants, or to the soil or water in which they are growing, with a pesticidally effective amount of compositions or compounds of formula I.

This invention also provides a method for treating, controlling, preventing or protecting animals against infestation or infection by parasites which comprises orally, topically or parenterally administering or applying to the animals a parasiticidally effective amount of compositions or compounds of formula I.

In spite of the commercial insecticides, acaricides and nematicides available today, damage to crops, both growing and harvested, caused by insects and nematodes still occurs. Therefore, there is continuing need to develop new and more effective insecticidal, acaricidal and nematicidal agents.

It was therefore an object of the present invention to provide new pesticidal compositions, new compounds and new methods for the control of insects, acarids or nematodes and of protecting growing plants from attack or infestation by insects, arachnids or nematodes.

We have found that these objects are achieved by the compositions and the compounds of formula I. Furthermore, we have found processes for preparing the compounds of formula I.

Resorcine derivates exhibiting pesticidal activity have been described in various documents: WO 03/042147, WO 04/098283, WO 04/098284, WO 04/099105, WO 04/052816, WO 04/113273, WO 04/020455, WO 04/002943, and WO 05/019147. These documents disclose resorcine derivatives which carry substituents in the 4-position of the resorcine ring. The resorcine derivates of the present invention are unsubstituted in the 4-position of the resorcine ring.

In WO 06/047438, which has been published after the priority date of this application, resorcine derivatives are disclosed which may be unsubstituted in the 4-position of the resorcine ring. Only two examples are given for such compounds (compounds 1 and 3), both of which are not subject to the present invention. WO 06/047438 clearly teaches 4-substituted resorcine derivatives as preferred.

Chinone derivatives exhibiting pesticidal activity have been described in EP-A 785923, JP 401128929, U.S. Pat. No. 5,922,880, WO96/04228, WO 96/11909, and WO 04/099197. In chinones, the two oxygen atoms are bonded to the phenylring in the 1,4-positions, whereas in resorcine derivates the oxygen atoms are in the 1,3-positions of the phenyl ring.

Generally, resorcine derivatives of formula (I) can be obtained by reaction of compounds (II) with compounds (III), wherein the variables in the compounds (II), (III) and (I) have the meaning as defined above for compounds I:

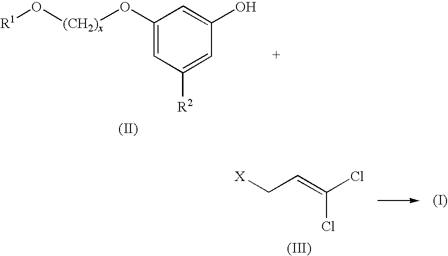

Generally, the reaction is carried out under the conditions of a nucleophilic replacement as described in literature known in the art (e.g. Organikum, VEB Berlin 1988, page 198 ff; JP 5718646; J. Am. Chem. Soc. 1956, 78, p. 6101, or in references cited in these references).

The reaction is preferably carried out under basic conditions. Suitable bases are, for example, alkali hydroxides, alkali hydrogen carbonates, alkali carbonates, alkaline earth metal hydroxides, carbonates or bicarbonates, or else alkali hydrides such as sodium hydride, amines such as triethylamine, diisopropylethylamine, pyridine or lutidine or lithiumamides such as lithiumhexamethyldisilazid or lithiumdiisopropylamine.

The amount of the base that can be used in the reaction is usually 0.9 to 5 moles relative to 1 mole of compound (II).

Suitable leaving groups X in compound (III) are halogen, preferably chloro, bromo or iodo, alkylcarbonylate, benzoate, alkylsulfonate, haloalkylsulfonate or arylsulfonate, most preferably chloro.

The reaction is advantageously carried out in an aprotic solvent such as dichloromethane, chloroform, carbon tetrachloride, benzene, toluene, diethyl ether or tetrahydrofurane, dimethylformamid, or mixtures of these solvents, in a temperature range between 0° C. and 100° C., preferably between 20° C. and 80° C.

If not commercially available, compounds (III) can be obtained according to literature procedures readily available to the artisan.

Compounds (II) can be prepared from reaction of compounds (VI) with alcohols (V) and subsequent cleavage of the protection group PG, wherein the variables in the compounds (VI) and (V) have the meaning as defined above for compounds I:

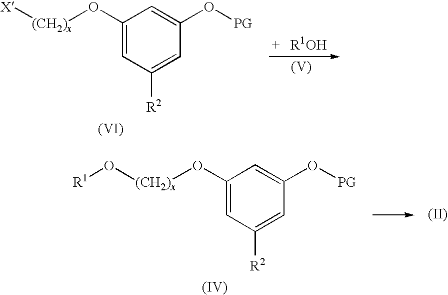

Generally, the reaction of compounds (VI) with alcohols (V) to compounds (IV) is carried out under the conditions of a nucleophilic replacement as described above for the alkylation of compounds (II). For those skilled in the art appropriate conditions can be found in R. C. Larock “Comprehensive organic transformations”, VCH Verlag, 1989, p. 445 ff.

The reaction is preferably carried out under basic conditions. Suitable bases are, for example, alkali hydroxides, alkali hydrogen carbonates, alkali carbonates, alkaline earth metal hydroxides, carbonates or bicarbonates, amines such as triethylamine or diisopropylethylamine, or lithiumamides such as lithiumhexamethyldisilazid or lithiumdiisopropylamine.

The stoechiometry of the reactants (VI) and (V) ranges from 0.8 to 1.5. The amount of the base that can be used in the reaction is usually 0.8 to 5 moles relative to 1 mole of compound (VI).

Suitable leaving groups X′ in compound (VI) are halogen, preferably chloro, bromo or iodo, alkylsulfonate, haloalkylsulfonate or arylsulfonate, most preferably chloro.

The reaction is advantageously carried out in a polar aprotic solvent such as dichloromethane, chloroform, carbon tetrachloride, benzene, toluene, diethyl ether or tetrahydrofurane, dimethylformamid, or mixtures of these solvents, in a temperature range between 0° C. and 100° C., preferably between 20° C. and 80° C.

The protection group PG may be any protection group that is stabile under basic reaction conditions, such as methyl, benzyl, benzyloxycarbonyl, methoxymethyl, or else as described in the appropriate literature. The conditions for the cleavage of the protection group PG may vary depending on the type of protection group and comprise hydrogenolytical, acid catalyzed or transition metal catalyzed methods. They are described in the art, e.g. in “Protecting groups”, Kocienski Thieme Verlag 1994, or in “Protective groups in organic synthesis” (Greene Wuts, Wiley, 1999).

If not commercially available, alcohols (V) can be prepared according to methods described in the art, for example in Joule, Mills, “Heterocyclic Chemistry”, Chapman+Hill 2000; Katritzky, Rees “Comprehensive Heterocyclic Chemistry”, Vol. 1-8, Pergamon Press 1984; “The Chemistry of Heterocyclic Compounds”. Wiley, Vol. 14 (1974), Vol. 29 (1974), 34 (1979), 37 (1981), 41 (1982), 49 (1991) oder Houben Weyl, “The Science of Synthesis”, Vol 9-22, Thieme 2005; or in references cited therein.

Compounds of formula (VI) can be obtained by reaction of compounds (VIII) with compounds (VII), wherein the variables in these compounds have the meaning as defined above for compounds I and PG is a protection group as defined above:

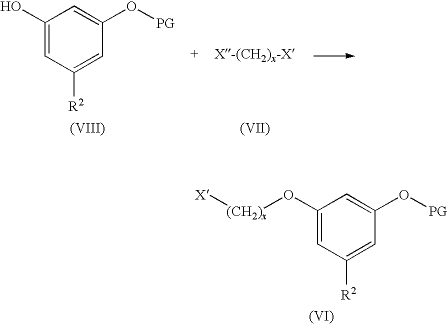

The reaction is carried out under the conditions of a nucleophilic replacement as described above for the alkylation of compounds (II).

Suitable leaving groups X′ and X″ in compound (VII) are each independently halogen, preferably chloro, bromo or iodo, alkylcarbonylate, benzoate, alkylsulfonate, haloalkylsulfonate or arylsulfonate. Preferably, X′ is chloro or bromo and X″ is chloro or bromo.

If not commercially available, resorcine derivatives of formula (VIII) and compounds (VII) can be obtained according to literature procedures known in the art, e.g by cleavage of the methoxy group in the commercially available dimethylethers.

According to a further embodiment of the invention, compound (IV) can be obtained by in a first step reacting compounds (VI) with compounds R′COOH in the presence of a base to give compounds (IX); which in a second step are reacted with compounds (V) in the presence of a base to give compounds (X); which in a third step are reacted with compounds (XI) to give compounds (IV), wherein the variables in these compounds have the meaning as defined above:

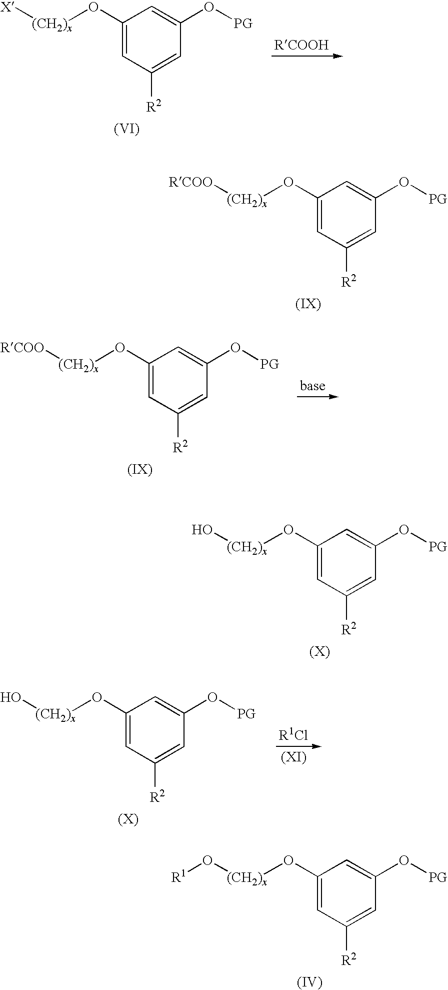

In a first step, compounds (VI) are converted to the alcohol compounds (X) e.g by reaction to the ester compounds (IX) and subsequent saponification of this ester with a base. The esters (IX) are formed by treating compounds (VI) with a carboxylic acid salt of aromatic or aliphatic acids R′COOH, eg. benzoic acid, acetic acid or the like, which are formed in situ in an aprotic solvent. The salt is formed with alkali or earth alkaline metall hydroxides, alkali hydrogen carbonates, alkali carbonates, or amine bases such as triethylamine or diisopropylethylamine, alkyl lithium bases such as butyllithium, or lithium amides such as lithiumdiisopropylamine. The amount of acid salt uses ranges from 0.9 to 3 molar equivalents relative to compounds (VI).

Suitable solvents are aprotic solvent like THF, methylenechlorid, toluene, acetone, dimethylformamide, N-methylpyrrolidone and the like. The reaction is performed in a temperature range from 0 to 100° C., preferably 20 to 80° C.

The basic saponification of this intermediate ester (IX) is carried out according to standard procedures know to those skilled in the art, e.g. as in Kocienski, “Protecting groups”, Thieme 1994, p. 22 ff.

The alcohol compounds (X) are reacted with compounds R¹CI (XI) to form compounds (IV) under the conditions of a nucleophilic replacement as described above for the reaction of compounds (VI) with alcohols (V) to compounds (IV).

The protection group PG is as defined above.

If not commercially available, compounds (XI) can be prepared according to methods described in the art, for example in Joule, Mills, “Heterocyclic Chemistry”, Chapman+Hill 2000; Katritzky, Rees “Comprehensive Heterocyclic Chemistry”, Vol. 1-8, Pergamon Press 1984; “The Chemistry of Heterocyclic Compounds”. Wiley, Vol. 14 (1974), Vol. 29 (1974), 34 (1979), 37 (1981), 41 (1982), 49 (1991) oder Houben Weyl, “The Science of Synthesis”, Vol 9-22, Thieme 2005; or in references cited therein.

The intermediates of formula (IV), (VI), (IX) and (X) are novel. These are also subject of this invention.

After completion of the reaction, the compound of formula (I) can be isolated by employing conventional methods such as adding the reaction mixture to water, extracting with an organic solvent, concentrating the extract an the like. The isolated compound (I) can be purified by a technique such as chromatography, recrystallization and the like, if necessary.

If individual compounds I are not obtainable by the routes described above, they can be prepared by derivatization of other compounds I or by customary modifications of the synthesis routes described.

The preparation of the compounds of formula I may lead to them being obtained as isomer mixtures (stereoisomers, enantiomers). If desired, these can be resolved by the methods customary for this purpose, such as crystallization or chromatography, also on optically active adsorbate, to give the pure isomers.

Agronomically acceptable salts of the compounds I can be formed in a customary manner, e.g. by reaction with an acid of the anion in question.

In this specification and in the claims, reference will be made to a number of terms that shall be defined to have the following meanings:

“Salt” as used herein includes adducts of compounds I with maleic acid, dimaleic acid, fumaric acid, difumaric acid, methane sulfenic acid, methane sulfonic acid, and succinic acid. Moreover, included as “salts” are those that can form with, for example, amines, metals, alkaline earth metal bases or quaternary ammonium bases, including zwitterions. Suitable metal and alkaline earth metal hydroxides as salt formers include the salts of barium, aluminum, nickel, copper, manganese, cobalt zinc, iron, silver, lithium, sodium, potassium, magnesium or calcium. Additional salt formers include chloride, sulfate, acetate, carbonate, hydride, and hydroxide. Desirable salts include adducts of compounds I with maleic acid, dimaleic acid, fumaric acid, difumaric acid, and methane sulfonic acid.

“Halogen” will be taken to mean fluoro, chloro, bromo and iodo.

The term “alkyl” as used herein refers to a branched or unbranched saturated hydrocarbon group having 1 to 6 carbon atoms, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

The term “haloalkyl” as used herein refers to a straight-chain or branched alkyl group having 1 to 6 carbon atoms (as mentioned above), where some or all of the hydrogen atoms in these groups may be replaced by halogen atoms as mentioned above, for example C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl;

Similarly, “alkoxy” and “alkylthio” refer to straight-chain or branched alkyl groups having 1 to 6 carbon atoms (as mentioned above) bonded through oxygen or sulfur linkages, respectively, at any bond in the alkyl group. Examples include methoxy, ethoxy, propoxy, isopropoxy, methylthio, ethylthio, propylthio, isopropylthio, and n-butylthio.

Similarly, “alkylsulfinyl” and “alkylsulfonyl” refer to straight-chain or branched alkyl groups having 1 to 6 carbon atoms (as mentioned above) bonded through —S(═O)— or —S(═O)₂-linkages, respectively, at any bond in the alkyl group. Examples include methylsulfinyl and methylsulfonyl.

Similarly, “alkylamino” refers to a nitrogen atom which carries 1 or 2 straight-chain or branched alkyl groups having 1 to 6 carbon atoms (as mentioned above) which may be the same or different. Examples include methylamino, dirmethylamino, ethylamino, diethylamino, methylethylamino, isopropylamino, or methylisopropylamino.

The term “alkenyl” as used herein intends a branched or unbranched unsaturated hydrocarbon group having 2 to 10 carbon atoms and a double bond in any position, for example C₂-C₆-alkenyl such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

The term “alkynyl” as used herein refers to a branched or unbranched unsaturated hydrocarbon group having 2 to 10 carbon atoms and containing at least one triple bond, such as ethynyl, propynyl, 1-butynyl, 2-butynyl, and the like.

A 5- or 6-membered heteroaromatic ring which contains 1 to 3 heteroatoms selected from oxygen, nitrogen and sulfur may be a 5-membered heteroaromatic ring containing 1 nitrogen atom and 0 to 2 further heteroatoms independently selected from oxygen, nitrogen and sulfur, such as pyrrol, pyrazol, imidazol, triazol, oxazol, isoxazol, oxadiazol, thiazol, isothiazol, thiodiazol; or a 5-membered heteroaromatic ring containing 1 heteroatom selected from oxygen and sulfur, such as furane or thiophen; or a 6-membered heteroaromatic ring containing 1 nitrogen atom and 0 to 2 further heteroatoms independently selected from oxygen, nitrogen and sulfur, preferably from nitrogen, such as pyridine, pyrazine, pyrimidine, pyridazine or triazine.

A 5- to 6-membered saturated, partially unsaturated or aromatic heterocyclic ring which may contain 1 to 3 heteroatoms selected from oxygen, nitrogen and sulfur is e.g. pyridine, pyrimidine, (1,2,4)-oxadiazole, (1,3,4)-oxadiazole, pyrrole, furan, thiophene, oxazole, thiazole, imidazole, pyrazole, isoxazole, 1,2,4-triazole, tetrazole, pyrazine, pyridazine, oxazoline, thiazoline, tetrahydrofuran, tetrahydropyran, morpholine, piperidine, piperazine, pyrroline, pyrrolidine, oxazolidine, thiazolidine. Preferably, this ring system is selected from pyridine, pyrimidine, (1,2,4)-oxadiazole, (1,3,4)-oxadiazole, pyrrole, furan, thiophene, oxazole, thiazole, imidazole, pyrazole, isoxazole, 1,2,4-triazole, tetrazole, pyrazine, pyridazine, oxazoline, thiazoline, tetrahydropyran, morpholine, piperidine, piperazine, pyrroline, pyrrolidine, oxazolidine, thiazolidine. Most preferably, this ring system is dioxolan, furan, oxazol, or thiazol.

A 5- to 7-membered saturated or partially unsaturated heterocyclic ring which may contain 1 to 3 heteroatoms selected from oxygen, sulfur and nitrogen is e.g. a 5- to 7-membered heteroaromatic ring containing 1 nitrogen atom and 0 or 1 further heteroatoms independently selected from oxygen and nitrogen, such as morpholine, piperazin, piperidine, or pyrrolidine.

A 5- or 6-membered heteroaromatic ring which contains 1 to 4 heteroatoms selected from oxygen, nitrogen and sulfur may be a 5-membered heteroaromatic ring containing 1 nitrogen atom and 0 to 2 further heteroatoms independently selected from oxygen, nitrogen and sulfur, preferably from oxygen and nitrogen, such as pyrrol, pyrazol, imidazol, triazol, oxazol, isoxazol, oxadiazol, thiazol, isothiazol, thiodiazol; or a 5-membered heteroaromatic ring containing 1 heteroatom selected from oxygen and sulfur, such as furane or thiophen; or a 6-membered heteroaromatic ring containing 1 nitrogen atom and 0 to 2 further heteroatoms independently selected from oxygen, nitrogen and sulfur, preferably from oxygen and nitrogen, such as pyridine, pyrazine, pyrimidine, pyridazine or triazine.

Cycloalkyl: monocyclic 3- to 6-, 8- or 10-membered saturated carbon atom rings, e.g. C₃-C₈-cycloalkyl such as cyclopropyl, cyclobutyl, cyclopentyl, cyclohexyl, cycloheptyl, or cyclooctyl.

With respect to the intended use of the compounds of formula I, particular preference is given to the following meanings of the substituents, in each case on their own or in combination:

A compound of formula I wherein x is 2, 3, 4, 5, 6 or 7, preferably 3, 4, 5 or 6.

A compound of formula I wherein R¹ is phenyl or a 5- to 6-membered heteroaromatic ring which may contain 1 to 3 heteroatoms selected from 1 to 3 nitrogen atoms or 1 to 2 nitrogen atoms and one oxygen or one sulfur atom, wherein phenyl or the heteroaromatic ring may be fused to a ring selected from phenyl and a 5- to 6-membered saturated, partially unsaturated or aromatic heterocyclic ring which may contain 1 to 3 heteroatoms selected from oxygen, nitrogen and sulfur.

A compound of formula I wherein R¹ is phenyl or a 5- to 6-membered heteroaromatic ring which may contain 1 to 3 heteroatoms selected from 1 to 3 nitrogen atoms or 1 to 2 nitrogen atoms and one oxygen or one sulfur atom, wherein phenyl or the heteroaromatic ring may be fused to a ring selected from phenyl and a 5- to 6-membered partially unsaturated or aromatic heterocyclic ring which may contain 1 to 3 heteroatoms selected from oxygen, nitrogen and sulfur.

A compound of formula I wherein R¹ is phenyl or a 5- to 6-membered heteroaromatic ring which may contain 1 to 3 heteroatoms selected from 1 to 3 nitrogen atoms or 1 to 2 nitrogen atoms and one oxygen or one sulfur atom, wherein phenyl or the heteroaromatic ring may be fused to a ring selected from phenyl and a 5- to 6-membered saturated, partially unsaturated or aromatic heterocyclic ring which may contain 1 to 3 heteroatoms selected from oxygen, nitrogen and sulfur. A compound of formula I wherein R¹ is phenyl or a 5- to 6-membered heteroaromatic ring which may contain 1 to 3 heteroatoms selected from 1 to 3 nitrogen atoms or 1 to 2 nitrogen atoms and one oxygen or one sulfur atom.

A compound of formula I wherein R¹ is substituted by any combination of 1 to 4 groups, preferably 1 or 3 groups R³.

Compounds of formula I wherein R¹ is phenyl or a 5- to 6-membered heteroaromatic ring selected from pyridyl, pyrimidyl, pyridazinyl, triazinyl, pyrazolyl, imidazolyl, thiazolyl, isothiazolyl, oxazolyl, isoxazolyl, oxdiazolyl, thiadiazolyl, and triazinyl, especially pyridyl.

Compounds of formula I wherein R¹ is phenyl, pyridyl, or phenyl fused to a 5-membered saturated, partially unsaturated or aromatic heterocyclic ring which may contain 1 to 3 heteroatoms, preferably 1 to 2 heteroatoms, selected from oxygen, nitrogen and sulfur.

Compounds of formula I wherein R¹ is phenyl, pyridyl, or phenyl fused to oxazole, thiazole, dioxolan, tetrahydrofuran or furanyl.

Compounds of formula I wherein R¹ is phenyl, pyridyl, or phenyl fused to oxazole, thiazole, dioxolan or furanyl.

Compounds of formula I wherein R¹ is a 5- to 6-membered heteroaromatic ring which may contain 1 to 3 heteroatoms selected from 1 to 3 nitrogen atoms or 1 to 2 nitrogen atoms and one oxygen or one sulfur atom, which may be fused to phenyl.

Preferred are compounds of formula I wherein R¹ is phenyl.

Preferred are compounds of formula I wherein R¹ is phenyl or 2-pyridyl.

Also, preferred are compounds of formula I wherein R¹ is 1, 2, 3, or 4 fold, preferably 1 or 3 fold, most preferably 1 fold, substituted pyridyl, preferably 2-pyridyl. The substituents R³ are then preferably selected from halogen, C₁-C₆-alkyl and C₁-C₆-haloalkyl, more preferably from halogen, C₁-C₄-alkyl and C₁-C₄-haloalkyl, even more preferably from fluorine, chlorine, methyl and trifluoromethyl, most preferably chlorine and trifluoromethyl.

A compound of formula I wherein R² is halogen, preferably chlorine or fluorine, methyl, or trifluoromethyl.

A compound of formula I wherein R² is halogen, preferably chlorine or fluorine.

A compound of formula I wherein R³ is halogen, C₁-C₆-alkyl, C₂-C₆-alkenyl, C₂-C₆-alkynyl, C₃-C₆-cycloalkyl, C₁-C₆-alkoxy, C₂-C₆-alkenyloxy, C₃-C₆-alkynyloxy, C₁-C₆-haloalkyl, C₂-C₆-haloalkenyl, C₂-C₆-haloalkynyl, C₁-C₆-haloalkoxy, C₂-C₆-haloalkenyloxy, C₃-C₆-haloalkynyloxy, or phenyl or a 5- to 7-membered saturated or partially unsaturated heterocyclic ring which may contain 1 to 3 heteroatoms selected from oxygen, sulfur or nitrogen or a 5- to 6-membered heteraromatic ring system which may contain 1 to 4 heteroatoms selected from oxygen, nitrogen and sulfur, which phenyl and which heteroaromatic ring may be bonded via an oxygen or a sulfur atom, wherein the above groups R³ are unsubstituted, or the hydrogen atoms in these groups may all or in part be replaced with any combination of groups selected from R^{a}, or —C(=G)R^{a}, —C(=G)OR^{a}, —C(=G)NR^{a}₂, —C(═NOR^{a})R^{a}, —C(═NOR^{a})NR^{a}₂, —NR^{a}C(=G)R^{a}, —NR^{a}C(=G)OR^{a}, —NR^{a}—C(=G)NR^{a}₂, —NR^{a}[(C=G)NR^{a}₂, —NR^{a}SO₂R^{a}, —S(═O)R^{a}, —S(═O)₂R^{a}, —SO₂OR^{a}, or —OSO₂R^{a}.

A compound of formula I wherein R³ is halogen, C₁-C₆-alkyl, C₂-C₆-alkenyl, C₂-C₆-alkynyl, C₁-C₆-alkoxy, C₂-C₆-alkenyloxy, C₃-C₆-alkynyloxy, C₁-C₆-haloalkyl, C₂-C₆-haloalkenyl, C₁-C₆-haloalkoxy, or C₂-C₆-haloalkenyloxy.

A compound of formula I wherein R³ is halogen, preferably chlorine or fluorine, cyano, C₁-C₄-alkyl, or C₁-C₄-haloalkyl, most preferably chlorine or trifluoromethyl

Especially preferred are resorcine derivative compounds of formula I wherein R¹ is selected from a ring system Q of table A.

TABLE A

| No. | R¹ | Subst. |
|---|---|---|
| Q.1 | 2-pyridyl | — |
| Q.2 | 2-pyridyl | 3 |
| Q.3 | 2-pyridyl | 4 |
| Q.4 | 2-pyridyl | 5 |
| Q.5 | 2-pyridyl | 6 |
| Q.6 | 2-pyridyl | 3.5 |
| Q.7 | 2-pyridyl | 3.4 |
| Q.8 | 2-pyridyl | 4.5 |
| Q.9 | 2-pyridyl | 5.6 |
| Q.10 | 2-pyridyl | 3.6 |
| Q.11 | 2-pyridyl | 4.6 |
| Q.12 | 3-pyridyl | — |
| Q.13 | 3-pyridyl | 2 |
| Q.14 | 3-pyridyl | 4 |
| Q.15 | 3-pyridyl | 5 |
| Q.16 | 3-pyridyl | 6 |
| Q.17 | 3-pyridyl | 4.6 |
| Q.18 | 3-pyridyl | 5.6 |
| Q.19 | 3-pyridyl | 2.6 |
| Q.20 | 3-pyridyl | 2.5 |
| Q.21 | 3-pyridyl | 4.5 |
| Q.22 | 4-pyridyl | — |
| Q.23 | 4-pyridyl | 2 |
| Q.24 | 4-pyridyl | 3 |
| Q.25 | 4-pyridyl | 5 |
| Q.26 | 4-pyridyl | 6 |
| Q.27 | 4-pyridyl | 3.6 |
| Q.28 | 4-pyridyl | 2.5 |
| Q.29 | 4-pyridyl | 2.6 |
| Q.30 | 3-pyridazinyl | — |
| Q.31 | 3-pyridazinyl | 4 |
| Q.32 | 3-pyridazinyl | 5 |
| Q.33 | 3-pyridazinyl | 6 |
| Q.34 | 3-pyridazinyl | 4.6 |
| Q.35 | 3-pyridazinyl | 4.5 |
| Q.36 | 3-pyridazinyl | 5.6 |
| Q.37 | 4-pyridazinyl | — |
| Q.38 | 4-pyridazinyl | 3 |
| Q.39 | 4-pyridazinyl | 5 |
| Q.40 | 4-pyridazinyl | 6 |
| Q.41 | 4-pyridazinyl | 3.5 |
| Q.42 | 4-pyridazinyl | 3.6 |
| Q.43 | 4-pyridazinyl | 5.6 |
| Q.44 | 2-pyrimidinyl | — |
| Q.45 | 2-pyrimidinyl | 4 |
| Q.46 | 2-pyrimidinyl | 5 |
| Q.47 | 2-pyrimidinyl | 6 |
| Q.48 | 2-pyrimidinyl | 4.5 |
| Q.49 | 2-pyrimidinyl | 4.6 |
| Q.50 | 4-pyrimidinyl | — |
| Q.51 | 4-pyrimidinyl | 2 |
| Q.52 | 4-pyrimidinyl | 5 |
| Q.53 | 4-pyrimidinyl | 6 |
| Q.54 | 4-pyrimidinyl | 2.6 |
| Q.55 | 4-pyrimidinyl | 5.6 |
| Q.56 | 4-pyrimidinyl | 2.5 |
| Q.57 | 2-pyrazinyl | — |
| Q.58 | 2-pyrazinyl | 3 |
| Q.59 | 2-pyrazinyl | 5 |
| Q.60 | 2-pyrazinyl | 6 |
| Q.61 | 2-pyrazinyl | 3.5 |
| Q.62 | 2-pyrazinyl | 5.6 |
| Q.63 | 3-pyrazolyl | — |
| Q.64 | 3-pyrazolyl | 4 |
| Q.65 | 3-pyrazolyl | 5 |
| Q.66 | 3-pyrazolyl | 4.5 |
| Q.67 | 3-(1-methyl)pyrazolyl | — |
| Q.68 | 3-(1-methyl)pyrazolyl | 4 |
| Q.69 | 3-(1-methyl)pyrazolyl | 5 |
| Q.70 | 3-(1-methyl)pyrazolyl | 4.5 |
| Q.71 | 4-pyrazolyl | — |
| Q.72 | 4-pyrazolyl | 3 |
| Q.73 | 4-pyrazolyl | 5 |
| Q.74 | 4-pyrazolyl | 3.5 |
| Q.75 | 4-(1-methyl)pyrazolyl | — |
| Q.76 | 4-(1-methyl)pyrazolyl | 3 |
| Q.77 | 4-(1-methyl)pyrazolyl | 5 |
| Q.78 | 4-(1-methyl)pyrazolyl | 3.5 |
| Q.79 | 4-imidazolyl | — |
| Q.80 | 4-imidazolyl | 2 |
| Q.81 | 4-imidazolyl | 5 |
| Q.82 | 4-imidazolyl | 2.5 |
| Q.83 | 4-(1-methyl)imidazolyl | — |
| Q.84 | 4-(1-methyl)imidazolyl | 2 |
| Q.85 | 4-(1 -methyl)imidazolyl | 5 |
| Q.86 | 4-(1-methyl)imidazolyl | 2.5 |
| Q.87 | 2-imidazolyl | — |
| Q.88 | 2-imidazolyl | 4 |
| Q.89 | 2-imidazolyl | 5 |
| Q.90 | 2-imidazolyl | 4.5 |
| Q.91 | 2-(1-methyl)imidazolyl | — |
| Q.92 | 2-(1-methyl)imidazolyl | 4 |
| Q.93 | 2-(1-methyl)imidazolyl | 5 |
| Q.94 | 2-(1-methyl)imidazolyl | 4.5 |
| Q.95 | 2-oxazolyl | — |
| Q.96 | 2-oxazolyl | 4 |
| Q.97 | 2-oxazolyl | 5 |
| Q.98 | 2-oxazolyl | 4.5 |
| Q.99 | 4-oxazolyl | — |
| Q.100 | 4-oxazolyl | 2 |
| Q.101 | 4-oxazolyl | 5 |
| Q.102 | 4-oxazolyl | 2.5 |
| Q.103 | 5-oxazolyl | — |
| Q.104 | 5-oxazolyl | 2 |
| Q.105 | 5-oxazolyl | 4 |
| Q.106 | 5-oxazolyl | 2.4 |
| Q.107 | 2-thiazolyl | — |
| Q.108 | 2-thiazolyl | 4 |
| Q.109 | 2-thiazolyl | 5 |
| Q.110 | 2-thiazolyl | 4.5 |
| Q.111 | 4-thiazolyl | — |
| Q.112 | 4-thiazolyl | 2 |
| Q.113 | 4-thiazolyl | 5 |
| Q.114 | 4-thiazolyl | 2.5 |
| Q.115 | 5-thiazolyl | — |
| Q.116 | 5-thiazolyl | 2 |
| Q.117 | 5-thiazolyl | 4 |
| Q.118 | 5-thiazolyl | 2.4 |
| Q.119 | 3-isoxazolyl | — |
| Q.120 | 3-isoxazolyl | 4 |
| Q.121 | 3-isoxazolyl | 5 |
| Q.122 | 3-isoxazolyl | 4.5 |
| Q.123 | 5-isoxazolyl | — |
| Q.124 | 5-isoxazolyl | 3 |
| Q.125 | 5-isoxazolyl | 4 |
| Q.126 | 5-isoxazolyl | 3.4 |
| Q.127 | 4-isoxazolyl | — |
| Q.128 | 4-isoxazolyl | 3 |
| Q.129 | 4-isoxazolyl | 5 |
| Q.130 | 4-isoxazolyl | 3.5 |
| Q.131 | 3-isothiazolyl | — |
| Q.132 | 3-isothiazolyl | 4 |
| Q.133 | 3-isothiazolyl | 5 |
| Q.134 | 3-isothiazolyl | 4.5 |
| Q.135 | 5-isothiazolyl | — |
| Q.136 | 5-isothiazolyl | 3 |
| Q.137 | 5-isothiazolyl | 4 |
| Q.138 | 5-isothiazolyl | 3.4 |
| Q.139 | 4-isothiazolyl | — |
| Q.140 | 4-isothiazolyl | 3 |
| Q.141 | 4-isothiazolyl | 5 |
| Q.142 | 4-isothiazolyl | 3.5 |
| Q.143 | 2-oxadiazole | — |
| Q.144 | 2-oxadiazole | 5 |
| Q.145 | 2-thiadiazole | — |
| Q.146 | 2-thiadiazole | 5 |
| Q.147 | 2-furanyl | — |
| Q.148 | 2-furanyl | 3 |
| Q.149 | 2-furanyl | 4 |
| Q.150 | 2-furanyl | 5 |
| Q.151 | 3-furanyl | — |
| Q.152 | 3-furanyl | 2 |
| Q.153 | 3-furanyl | 4 |
| Q.154 | 3-furanyl | 5 |
| Q.155 | 2-thiophenyl | — |
| Q.156 | 2-thiophenyl | 3 |
| Q.157 | 2-thiophenyl | 4 |
| Q.158 | 2-thiophenyl | 5 |
| Q.159 | 3-thiophenyl | — |
| Q.160 | 3-thiophenyl | 2 |
| Q.161 | 3-thiophenyl | 4 |
| Q.162 | 3-thiophenyl | 5 |
| Q.163 | phenyl | — |
| Q.164 | phenyl | 2 |
| Q.165 | phenyl | 3 |
| Q.166 | phenyl | 4 |
| Q.167 | phenyl | 2.3 |
| Q.168 | phenyl | 2.4 |
| Q.169 | phenyl | 2.5 |
| Q.170 | phenyl | 2.6 |
| Q.171 | phenyl | 3.4 |
| Q.172 | phenyl | 3.5 |
| Q.173 | phenyl | 4.5 |
| Q.174 | 4-allyl-phenyl | — |
| Q.175 | 4-allyl-phenyl | 2 |
| Q.176 | 4-allyl-phenyl | 3 |
| Q.177 | 4-(2-cyanoallyl)- | — |
| | phenyl | |
| Q.178 | 4-(2-cyanoallyl)- | 2 |
| | phenyl | |
| Q.179 | 4-(2-cyanoallyl)- | 3 |
| | phenyl | |
| Q.180 | 4-(2,2-dicyanoallyl)- | — |
| | phenyl | |
| Q.181 | 4-(2,2-dicyanoallyl)- | 2 |
| | phenyl | |
| Q.182 | 4-(2,2-dicyanoallyl)- | 3 |
| | phenyl | |
| Q.183 | 2-benzothiazolyl | — |
| Q.184 | 2-benzothiazolyl | 4 |
| Q.185 | 2-benzothiazolyl | 5 |
| Q.186 | 2-benzothiazolyl | 6 |
| Q.187 | 2-benzothiazolyl | 7 |
| Q.188 | 2-benzooxazolyl | — |
| Q.189 | 2-benzooxazolyl | 4 |
| Q.190 | 2-benzooxazolyl | 5 |
| Q.191 | 2-benzooxazolyl | 6 |
| Q.192 | 2-benzooxazolyl | 7 |
| Q.193 | 4-benzothiazolyl | — |
| Q.194 | 4-benzothiazolyl | 2 |
| Q.195 | 5-benzothiazolyl | — |
| Q.196 | 5-benzothiazolyl | 2 |
| Q.197 | 6-benzothiazolyl | — |
| Q.198 | 6-benzothiazolyl | 2 |
| Q.199 | 7-benzothiazolyl | — |
| Q.200 | 7-benzothiazolyl | 2 |
| Q.201 | 4-benzooxazolyl | — |
| Q.202 | 4-benzooxazolyl | 2 |
| Q.203 | 5-benzooxazolyl | — |
| Q.204 | 5-benzooxazolyl | 2 |
| Q.205 | 6-benzooxazolyl | — |
| Q.206 | 6-benzooxazolyl | 2 |
| Q.207 | 7-benzooxazolyl | — |
| Q.208 | 7-benzooxazolyl | 2 |
| Q.209 | 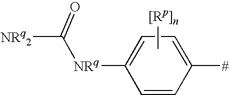 | 2 |
| Q.210 | 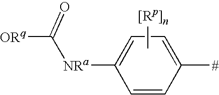 | 2 |
| Q.211 | 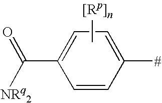 | 2 |
| Q.212 | 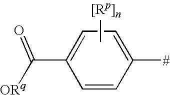 | 2 |

wherein
subst. means substitution pattern of the ring system and # denotes the binding site; the substitutents of the ring systems Q.1 to Q.65 and Q.72 to Q.87 preferably are each independently selected from fluorine, chlorine, bromine, cyano, methyl, methoxy, trifluoromethyl, trifluoromethoxy, trifluoromethylthio, S(═O)₂CH₃ and S(═O)₂CF₃; the substituents R^{q} in the ring systems Q.209 to Q.212 preferably are each independently selected from hydrogen, methyl, ethyl, isopropyl and allyl; the substituents R^{P} in the ring systems Q.209 to Q.212 preferably are each independently selected from hydrogen, fluorine, chlorine, bromine, methoxy, trifluoromethyl and trifluoromethoxy.

Preferred are compound of formula I wherein Q is selected from the group Q.2 to Q.11. Preferred substituents R³ are as described above, preferably chosen from the group halogen, preferably chlorine or fluorine, cyano, C₁-C₄-alkyl, or C₁-C₄-haloalkyl, most preferably chlorine or trifluoromethyl.

With respect to their use, particular preference is given to the compounds I compiled in the tables below. Moreover, the groups mentioned for a substituent in the tables are on their own, independently of the combination in which they are mentioned, a particularly preferred embodiment of the substituent in question.

### Table 1

Compounds of the formula I wherein R² denotes chlorine, x denotes 2 and the combination of R¹ and R³ in each case corresponds to a row of Table B.

### Table 2

Compounds of the formula I wherein R² denotes fluorine, x denotes 2 and the combination of R¹ and R³ in each case corresponds to a row of Table B.

### Table 3

Compounds of the formula I wherein R² denotes bromine, x denotes 2 and the combination of R¹ and R³ in each case corresponds to a row of Table B.

### Table 4

Compounds of the formula I wherein R² denotes cyano, x denotes 2 and the combination of R¹ and R³ in each case corresponds to a row of Table B.

### Table 5

Compounds of the formula I wherein R² denotes methyl, x denotes 2 and the combination of R¹ and R³ in each case corresponds to a row of Table B.

### Table 6

Compounds of the formula I wherein R² denotes trifluoromethyl, x denotes 2 and the combination of R¹ and R³ in each case corresponds to a row of Table B.

### Table 7

Compounds of the formula I wherein R² denotes methoxy, x denotes 2 and the combination of R¹ and R³ in each case corresponds to a row of Table B.

### Table 8

Compounds of the formula I wherein R² denotes chlorine, x denotes 3 and the combination of R¹ and R³ in each case corresponds to a row of Table B.

### Table 9

Compounds of the formula I wherein R² denotes fluorine, x denotes 3 and the combination of R¹ and R³ in each case corresponds to a row of Table B.

### Table 10

Compounds of the formula I wherein R² denotes bromine, x denotes 3 and the combination of R¹ and R³ in each case corresponds to a row of Table B.

### Table 11

Compounds of the formula I wherein R² denotes cyano, x denotes 3 and the combination of R¹ and R³ in each case corresponds to a row of Table B.

### Table 12

Compounds of the formula I wherein R² denotes methyl, x denotes 3 and the combination of R¹ and R³ in each case corresponds to a row of Table B.

### Table 13

Compounds of the formula I wherein R² denotes trifluoromethyl, x denotes 3 and the combination of R¹ and R³ in each case corresponds to a row of Table B.

### Table 14

Compounds of the formula I wherein R² denotes methoxy, x denotes 3 and the combination of R¹ and R³ in each case corresponds to a row of Table B.

### Table 15

Compounds of the formula I wherein R² denotes chlorine, x denotes 4 and the combination of R¹ and R³ in each case corresponds to a row of Table B.

### Table 16

Compounds of the formula I wherein R² denotes fluorine, x denotes 4 and the combination of R¹ and R³ in each case corresponds to a row of Table B.

### Table 17

Compounds of the formula I wherein R² denotes bromine, x denotes 4 and the combination of R¹ and R³ in each case corresponds to a row of Table B.

### Table 18

Compounds of the formula I wherein R² denotes cyano, x denotes 4 and the combination of R¹ and R³ in each case corresponds to a row of Table B.

### Table 19

Compounds of the formula I wherein R² denotes methyl, x denotes 4 and the combination of R¹ and R³ in each case corresponds to a row of Table B.

### Table 20

Compounds of the formula I wherein R² denotes trifluoromethyl, x denotes 4 and the combination of R¹ and R³ in each case corresponds to a row of Table B.

### Table 21

Compounds of the formula I wherein R² denotes methoxy, x denotes 4 and the combination of R¹ and R³ in each case corresponds to a row of Table B.

### Table 22

Compounds of the formula I wherein R² denotes chlorine, x denotes 5 and the combination of R¹ and R³ in each case corresponds to a row of Table B.

### Table 23

Compounds of the formula I wherein R² denotes fluorine, x denotes 5 and the combination of R¹ and R³ in each case corresponds to a row of Table B.

### Table 24

Compounds of the formula I wherein R² denotes bromine, x denotes 5 and the combination of R¹ and R³ in each case corresponds to a row of Table B.

### Table 25

Compounds of the formula I wherein R² denotes cyano, x denotes 5 and the combination of R¹ and R³ in each case corresponds to a row of Table B.

### Table 26

Compounds of the formula I wherein R² denotes methyl, x denotes 5 and the combination of R¹ and R³ in each case corresponds to a row of Table B.

### Table 27

Compounds of the formula I wherein R² denotes trifluoromethyl, x denotes 5 and the combination of R¹ and R³ in each case corresponds to a row of Table B.

### Table 28

Compounds of the formula I wherein R² denotes methoxy, x denotes 5 and the combination of R¹ and R³ in each case corresponds to a row of Table B.

### Table 29

Compounds of the formula I wherein R² denotes chlorine, x denotes 6 and the combination of R¹ and R³ in each case corresponds to a row of Table B.

### Table 30

Compounds of the formula I wherein R² denotes fluorine, x denotes 6 and the combination of R¹ and R³ in each case corresponds to a row of Table B.

### Table 31

Compounds of the formula I wherein R² denotes bromine, x denotes 6 and the combination of R¹ and R³ in each case corresponds to a row of Table B.

### Table 32

Compounds of the formula I wherein R² denotes cyano, x denotes 6 and the combination of R¹ and R³ in each case corresponds to a row of Table B.

### Table 33

Compounds of the formula I wherein R² denotes methyl, x denotes 6 and the combination of R¹ and R³ in each case corresponds to a row of Table B.

### Table 34

Compounds of the formula I wherein R² denotes trifluoromethyl, x denotes 6 and the combination of R¹ and R³ in each case corresponds to a row of Table B.

### Table 35

Compounds of the formula I wherein R² denotes methoxy, x denotes 6 and the combination of R¹ and R³ in each case corresponds to a row of Table B.

### Table 36

Compounds of the formula I wherein R² denotes chlorine, x denotes 7 and the combination of R¹ and R³ in each case corresponds to a row of Table B.

### Table 37

Compounds of the formula I wherein R² denotes fluorine, x denotes 7 and the combination of R¹ and R³ in each case corresponds to a row of Table B.

### Table 38

Compounds of the formula I wherein R² denotes bromine, x denotes 7 and the combination of R¹ and R³ in each case corresponds to a row of Table B.

### Table 39

Compounds of the formula I wherein R² denotes cyano, x denotes 7 and the combination of R¹ and R³ in each case corresponds to a row of Table B.

### Table 40

Compounds of the formula I wherein R² denotes methyl, x denotes 7 and the combination of R¹ and R³ in each case corresponds to a row of Table B.

### Table 41

Compounds of the formula I wherein R² denotes trifluoromethyl, x denotes 7 and the combination of R¹ and R³ in each case corresponds to a row of Table B.

### Table 42

Compounds of the formula I wherein R² denotes methoxy, x denotes 7 and the combination of R¹ and R³ in each case corresponds to a row of Table B.

TABLE B

| No. | R¹ | R³ |
|---|---|---|
| I-1 | Q.1 | — |
| I-2 | Q.2 | 3-F |
| I-3 | Q.2 | 3-Cl |
| I-4 | Q.2 | 3-Br |
| I-5 | Q.2 | 3-CH₃ |
| I-6 | Q.2 | 3-OCH₃ |
| I-7 | Q.2 | 3-CF₃ |
| I-8 | Q.3 | 4-F |
| I-9 | Q.3 | 4-Cl |
| I-10 | Q.3 | 4-Br |
| I-11 | Q.3 | 4-CH₃ |
| I-12 | Q.3 | 4-OCH₃ |
| I-13 | Q.3 | 4-CF₃ |
| I-14 | Q.4 | 5-F |
| I-15 | Q.4 | 5-Cl |
| I-16 | Q.4 | 5-Br |
| I-17 | Q.4 | 5-CH₃ |
| I-18 | Q.4 | 5-OCH₃ |
| I-19 | Q.4 | 5-CF₃ |
| I-20 | Q.4 | 5-SO₂CH₃ |
| I-21 | Q.4 | 5-SCF₃ |
| I-22 | Q.4 | 5-SO₂CF₃ |
| I-23 | Q.4 | 5-CN |
| I-24 | Q.4 | 5-OCF₃ |
| I-25 | Q.5 | 6-F |
| I-26 | Q.5 | 6-Cl |
| I-27 | Q.5 | 6-Br |
| I-28 | Q.5 | 6-CH₃ |
| I-29 | Q.5 | 6-OCH₃ |
| I-30 | Q.5 | 6-CF₃ |
| I-31 | Q.5 | 6-SO₂CH₃ |
| I-32 | Q.5 | 6-SCF₃ |
| I-33 | Q.5 | 6-SO₂CF₃ |
| I-34 | Q.5 | 6-CN |
| I-35 | Q.5 | 6-OCF₃ |
| I-36 | Q.6 | 3-F, 5-F |
| I-37 | Q.6 | 3-F, 5-Cl |
| I-38 | Q.6 | 3-F, 5-Br |
| I-39 | Q.6 | 3-F, 5-CH₃ |
| I-40 | Q.6 | 3-F, 5-OCH₃ |
| I-41 | Q.6 | 3-F, 5-CF₃ |
| I-42 | Q.6 | 3-F, 5-SO₂CH₃ |
| I-43 | Q.6 | 3-F, 5-SCF₃ |
| I-44 | Q.6 | 3-F, 5-SO₂CF₃ |
| I-45 | Q.6 | 3-F, 5-CN |
| I-46 | Q.6 | 3-F, 5-OCF₃ |
| I-47 | Q.6 | 3-Cl, 5-F |
| I-48 | Q.6 | 3-Cl, 5-Cl |
| I-49 | Q.6 | 3-Cl, 5-Br |
| I-50 | Q.6 | 3-Cl, 5-CH₃ |
| I-51 | Q.6 | 3-Cl, 5-OCH₃ |
| I-52 | Q.6 | 3-Cl, 5-CF₃ |
| I-53 | Q.6 | 3-Cl, 5-SO₂CH₃ |
| I-54 | Q.6 | 3-Cl, 5-SCF₃ |
| I-55 | Q.6 | 3-Cl, 5-SO₂CF₃ |
| I-56 | Q.6 | 3-Cl, 5-CN |
| I-57 | Q.6 | 3-Cl, 5-OCF₃ |
| I-58 | Q.6 | 3-Br, 5-F |
| I-59 | Q.6 | 3-Br, 5-Cl |
| I-60 | Q.6 | 3-Br, 5-Br |
| I-61 | Q.6 | 3-Br, 5-CH₃ |
| I-62 | Q.6 | 3-Br, 5-OCH₃ |
| I-63 | Q.6 | 3-Br, 5-CF₃ |
| I-64 | Q.6 | 3-Br, 5-SO₂CH₃ |
| I-65 | Q.6 | 3-Br, 5-SCF₃ |
| I-66 | Q.6 | 3-Br, 5-SO₂CF₃ |
| I-67 | Q.6 | 3-Br, 5-CN |
| I-68 | Q.6 | 3-Br, 5-OCF₃ |
| I-69 | Q.6 | 3-CH₃, 5-F |
| I-70 | Q.6 | 3-CH₃, 5-Cl |
| I-71 | Q.6 | 3-CH₃, 5-Br |
| I-72 | Q.6 | 3-CH₃, 5-CH₃ |
| I-73 | Q.6 | 3-CH₃, 5-OCH₃ |
| I-74 | Q.6 | 3-CH₃, 5-CF₃ |
| I-75 | Q.6 | 3-CH₃, 5-SO₂CH₃ |
| I-76 | Q.6 | 3-CH₃, 5-SCF₃ |
| I-77 | Q.6 | 3-CH₃, 5-SO₂CF₃ |
| I-78 | Q.6 | 3-CH₃, 5-CN |
| I-79 | Q.6 | 3-CH₃, 5-OCF₃ |
| I-80 | Q.6 | 3-OCH₃, 5-F |
| I-81 | Q.6 | 3-OCH₃, 5-Cl |
| I-82 | Q.6 | 3-OCH₃, 5-Br |
| I-83 | Q.6 | 3-OCH₃, 5-CH₃ |
| I-84 | Q.6 | 3-OCH₃, 5-OCH₃ |
| I-85 | Q.6 | 3-OCH₃, 5-CF₃ |
| I-86 | Q.6 | 3-OCH₃, 5-SO₂CH₃ |
| I-87 | Q.6 | 3-OCH₃, 5-SCF₃ |
| I-88 | Q.6 | 3-OCH₃, 5-SO₂CF₃ |
| I-89 | Q.6 | 3-OCH₃, 5-CN |
| I-90 | Q.6 | 3-OCH₃, 5-OCF₃ |
| I-91 | Q.6 | 3-CF₃, 5-F |
| I-92 | Q.6 | 3-CF₃, 5-Cl |
| I-93 | Q.6 | 3-CF₃, 5-Br |
| I-94 | Q.6 | 3-CF₃, 5-CH₃ |
| I-95 | Q.6 | 3-CF₃, 5-OCH₃ |
| I-96 | Q.6 | 3-CF₃, 5-CF₃ |
| I-97 | Q.6 | 3-CF₃, 5-SO₂CH₃ |
| I-98 | Q.6 | 3-CF₃, 5-SCF₃ |
| I-99 | Q.6 | 3-CF₃, 5-SO₂CF₃ |
| I-100 | Q.6 | 3-CF₃, 5-CN |
| I-101 | Q.6 | 3-CF₃, 5-OCF₃ |
| I-102 | Q.7 | 3-F, 4-F |
| I-103 | Q.7 | 3-F, 4-Cl |
| I-104 | Q.7 | 3-F, 4-Br |
| I-105 | Q.7 | 3-F, 4-CH₃ |
| I-106 | Q.7 | 3-F, 4-OCH₃ |
| I-107 | Q.7 | 3-F, 4-CF₃ |
| I-108 | Q.7 | 3-F, 4-SO₂CH₃ |
| I-109 | Q.7 | 3-F, 4-SCF₃ |
| I-110 | Q.7 | 3-F, 4-SO₂CF₃ |
| I-111 | Q.7 | 3-F, 4-CN |
| I-112 | Q.7 | 3-F, 4-OCF₃ |
| I-113 | Q.7 | 3-Cl, 4-F |
| I-114 | Q.7 | 3-Cl, 4-Cl |
| I-115 | Q.7 | 3-Cl, 4-Br |
| I-116 | Q.7 | 3-Cl, 4-CH₃ |
| I-117 | Q.7 | 3-Cl, 4-OCH₃ |
| I-118 | Q.7 | 3-Cl, 4-CF₃ |
| I-119 | Q.7 | 3-Cl, 4-SO₂CH₃ |
| I-120 | Q.7 | 3-Cl, 4-SCF₃ |
| I-121 | Q.7 | 3-Cl, 4-SO₂CF₃ |
| I-122 | Q.7 | 3-Cl, 4-CN |
| I-123 | Q.7 | 3-Cl, 4-OCF₃ |
| I-124 | Q.7 | 3-Br, 4-F |
| I-125 | Q.7 | 3-Br, 4-Cl |
| I-126 | Q.7 | 3-Br, 4-Br |
| I-127 | Q.7 | 3-Br, 4-CH₃ |
| I-128 | Q.7 | 3-Br, 4-OCH₃ |
| I-129 | Q.7 | 3-Br, 4-CF₃ |
| I-130 | Q.7 | 3-Br, 4-SO₂CH₃ |
| I-131 | Q.7 | 3-Br, 4-SCF₃ |
| I-132 | Q.7 | 3-Br, 4-SO₂CF₃ |
| I-133 | Q.7 | 3-Br, 4-CN |
| I-134 | Q.7 | 3-Br, 4-OCF₃ |
| I-135 | Q.7 | 3-CH₃, 4-F |
| I-136 | Q.7 | 3-CH₃, 4-Cl |
| I-137 | Q.7 | 3-CH₃, 4-Br |
| I-138 | Q.7 | 3-CH₃, 4-CH₃ |
| I-139 | Q.7 | 3-CH₃, 4-OCH₃ |
| I-140 | Q.7 | 3-CH₃, 4-CF₃ |
| I-141 | Q.7 | 3-CH₃, 4-SO₂CH₃ |
| I-142 | Q.7 | 3-CH₃, 4-SCF₃ |
| I-143 | Q.7 | 3-CH₃, 4-SO₂CF₃ |
| I-144 | Q.7 | 3-CH₃, 4-CN |
| I-145 | Q.7 | 3-CH₃, 4-OCF₃ |
| I-146 | Q.7 | 3-OCH₃, 4-F |
| I-147 | Q.7 | 3-OCH₃, 4-Cl |
| I-148 | Q.7 | 3-OCH₃, 4-Br |
| I-149 | Q.7 | 3-OCH₃, 4-CH₃ |
| I-150 | Q.7 | 3-OCH₃, 4-OCH₃ |
| I-151 | Q.7 | 3-OCH₃, 4-CF₃ |
| I-152 | Q.7 | 3-OCH₃, 4-SO₂CH₃ |
| I-153 | Q.7 | 3-OCH₃, 4-SCF₃ |
| I-154 | Q.7 | 3-OCH₃, 4-SO₂CF₃ |
| I-155 | Q.7 | 3-OCH₃, 4-CN |
| I-156 | Q.7 | 3-OCH₃, 4-OCF₃ |
| I-157 | Q.7 | 3-CF₃, 4-F |
| I-158 | Q.7 | 3-CF₃, 4-Cl |
| I-159 | Q.7 | 3-CF₃, 4-Br |
| I-160 | Q.7 | 3-CF₃, 4-CH₃ |
| I-161 | Q.7 | 3-CF₃, 4-OCH₃ |
| I-162 | Q.7 | 3-CF₃, 4-CF₃ |
| I-163 | Q.7 | 3-CF₃, 4-SO₂CH₃ |
| I-164 | Q.7 | 3-CF₃, 4-SCF₃ |
| I-165 | Q.7 | 3-CF₃, 4-SO₂CF₃ |
| I-166 | Q.7 | 3-CF₃, 4-CN |
| I-167 | Q.7 | 3-CF₃, 4-OCF₃ |
| I-168 | Q.8 | 4-F, 5-F |
| I-169 | Q.8 | 4-F, 5-Cl |
| I-170 | Q.8 | 4-F, 5-Br |
| I-171 | Q.8 | 4-F, 5-CH₃ |
| I-172 | Q.8 | 4-F, 5-OCH₃ |
| I-173 | Q.8 | 4-F, 5-CF₃ |
| I-174 | Q.8 | 4-F, 5-SO₂CH₃ |
| I-175 | Q.8 | 4-F, 5-SCF₃ |
| I-176 | Q.8 | 4-F, 5-SO₂CF₃ |
| I-177 | Q.8 | 4-F, 5-CN |
| I-178 | Q.8 | 4-F, 5-OCF₃ |
| I-179 | Q.8 | 4-Cl, 5-F |
| I-180 | Q.8 | 4-Cl, 5-Cl |
| I-181 | Q.8 | 4-Cl, 5-Br |
| I-182 | Q.8 | 4-Cl, 5-CH₃ |
| I-183 | Q.8 | 4-Cl, 5-OCH₃ |
| I-184 | Q.8 | 4-Cl, 5-CF₃ |
| I-185 | Q.8 | 4-Cl, 5-SO₂CH₃ |
| I-186 | Q.8 | 4-Cl, 5-SCF₃ |
| I-187 | Q.8 | 4-Cl, 5-SO₂CF₃ |
| I-188 | Q.8 | 4-Cl, 5-CN |
| I-189 | Q.8 | 4-Cl, 5-OCF₃ |
| I-190 | Q.8 | 4-Br, 5-F |
| I-191 | Q.8 | 4-Br, 5-Cl |
| I-192 | Q.8 | 4-Br, 5-Br |
| I-193 | Q.8 | 4-Br, 5-CH₃ |
| I-194 | Q.8 | 4-Br, 5-OCH₃ |
| I-195 | Q.8 | 4-Br, 5-CF₃ |
| I-196 | Q.8 | 4-Br, 5-SO₂CH₃ |
| I-197 | Q.8 | 4-Br, 5-SCF₃ |
| I-198 | Q.8 | 4-Br, 5-SO₂CF₃ |
| I-199 | Q.8 | 4-Br, 5-CN |
| I-200 | Q.8 | 4-Br, 5-OCF₃ |
| I-201 | Q.8 | 4-CH₃, 5-F |
| I-202 | Q.8 | 4-CH₃, 5-Cl |
| I-203 | Q.8 | 4-CH₃, 5-Br |
| I-204 | Q.8 | 4-CH₃, 5-CH₃ |
| I-205 | Q.8 | 4-CH₃, 5-OCH₃ |
| I-206 | Q.8 | 4-CH₃, 5-CF₃ |
| I-207 | Q.8 | 4-CH₃, 5-SO₂CH₃ |
| I-208 | Q.8 | 4-CH₃, 5-SCF₃ |
| I-209 | Q.8 | 4-CH₃, 5-SO₂CF₃ |
| I-210 | Q.8 | 4-CH₃, 5-CN |
| I-211 | Q.8 | 4-CH₃, 5-OCF₃ |
| I-212 | Q.8 | 4-OCH₃, 5-F |
| I-213 | Q.8 | 4-OCH₃, 5-Cl |
| I-214 | Q.8 | 4-OCH₃, 5-Br |
| I-215 | Q.8 | 4-OCH₃, 5-CH₃ |
| I-216 | Q.8 | 4-OCH₃, 5-OCH₃ |
| I-217 | Q.8 | 4-OCH₃, 5-CF₃ |
| I-218 | Q.8 | 4-OCH₃, 5-SO₂CH₃ |
| I-219 | Q.8 | 4-OCH₃, 5-SCF₃ |
| I-220 | Q.8 | 4-OCH₃, 5-SO₂CF₃ |
| I-221 | Q.8 | 4-OCH₃, 5-CN |
| I-222 | Q.8 | 4-OCH₃, 5-OCF₃ |
| I-223 | Q.8 | 4-CF₃, 5-F |
| I-224 | Q.8 | 4-CF₃, 5-Cl |
| I-225 | Q.8 | 4-CF₃, 5-Br |
| I-226 | Q.8 | 4-CF₃, 5-CH₃ |
| I-227 | Q.8 | 4-CF₃, 5-OCH₃ |
| I-228 | Q.8 | 4-CF₃, 5-CF₃ |
| I-229 | Q.8 | 4-CF₃, 5-SO₂CH₃ |
| I-230 | Q.8 | 4-CF₃, 5-SCF₃ |
| I-231 | Q.8 | 4-CF₃, 5-SO₂CF₃ |
| I-232 | Q.8 | 4-CF₃, 5-CN |
| I-233 | Q.8 | 4-CF₃, 5-OCF₃ |
| I-234 | Q.9 | 6-F, 5-F |
| I-235 | Q.9 | 6-F, 5-Cl |
| I-236 | Q.9 | 6-F, 5-Br |
| I-237 | Q.9 | 6-F, 5-CH₃ |
| I-238 | Q.9 | 6-F, 5-OCH₃ |
| I-239 | Q.9 | 6-F, 5-CF₃ |
| I-240 | Q.9 | 6-F, 5-SO₂CH₃ |
| I-241 | Q.9 | 6-F, 5-SCF₃ |
| I-242 | Q.9 | 6-F, 5-SO₂CF₃ |
| I-243 | Q.9 | 6-F, 5-CN |
| I-244 | Q.9 | 6-F, 5-OCF₃ |
| I-245 | Q.9 | 6-Cl, 5-F |
| I-246 | Q.9 | 6-Cl, 5-Cl |
| I-247 | Q.9 | 6-Cl, 5-Br |
| I-248 | Q.9 | 6-Cl, 5-CH₃ |
| I-249 | Q.9 | 6-Cl, 5-OCH₃ |
| I-250 | Q.9 | 6-Cl, 5-CF₃ |
| I-251 | Q.9 | 6-Cl, 5-SO₂CH₃ |
| I-252 | Q.9 | 6-Cl, 5-SCF₃ |
| I-253 | Q.9 | 6-Cl, 5-SO₂CF₃ |
| I-254 | Q.9 | 6-Cl, 5-CN |
| I-255 | Q.9 | 6-Cl, 5-OCF₃ |
| I-256 | Q.9 | 6-Br, 5-F |
| I-257 | Q.9 | 6-Br, 5-Cl |
| I-258 | Q.9 | 6-Br, 5-Br |
| I-259 | Q.9 | 6-Br, 5-CH₃ |
| I-260 | Q.9 | 6-Br, 5-OCH₃ |
| I-261 | Q.9 | 6-Br, 5-CF₃ |
| I-262 | Q.9 | 6-Br, 5-SO₂CH₃ |
| I-263 | Q.9 | 6-Br, 5-SCF₃ |
| I-264 | Q.9 | 6-Br, 5-SO₂CF₃ |
| I-265 | Q.9 | 6-Br, 5-CN |
| I-266 | Q.9 | 6-Br, 5-OCF₃ |
| I-267 | Q.9 | 6-CH₃, 5-F |
| I-268 | Q.9 | 6-CH₃, 5-Cl |
| I-269 | Q.9 | 6-CH₃, 5-Br |
| I-270 | Q.9 | 6-CH₃, 5-CH₃ |
| I-271 | Q.9 | 6-CH₃, 5-OCH₃ |
| I-272 | Q.9 | 6-CH₃, 5-CF₃ |
| I-273 | Q.9 | 6-CH₃, 5-SO₂CH₃ |
| I-274 | Q.9 | 6-CH₃, 5-SCF₃ |
| I-275 | Q.9 | 6-CH₃, 5-SO₂CF₃ |
| I-276 | Q.9 | 6-CH₃, 5-CN |
| I-277 | Q.9 | 6-CH₃, 5-OCF₃ |
| I-278 | Q.9 | 6-OCH₃, 5-F |
| I-279 | Q.9 | 6-OCH₃, 5-Cl |
| I-280 | Q.9 | 6-OCH₃, 5-Br |
| I-281 | Q.9 | 6-OCH₃, 5-CH₃ |
| I-282 | Q.9 | 6-OCH₃, 5-OCH₃ |
| I-283 | Q.9 | 6-OCH₃, 5-CF₃ |
| I-284 | Q.9 | 6-OCH₃, 5-SO₂CH₃ |
| I-285 | Q.9 | 6-OCH₃, 5-SCF₃ |
| I-286 | Q.9 | 6-OCH₃, 5-SO₂CF₃ |
| I-287 | Q.9 | 6-OCH₃, 5-CN |
| I-288 | Q.9 | 6-OCH₃, 5-OCF₃ |
| I-289 | Q.9 | 6-CF₃, 5-F |
| I-290 | Q.9 | 6-CF₃, 5-Cl |
| I-291 | Q.9 | 6-CF₃, 5-Br |
| I-292 | Q.9 | 6-CF₃, 5-CH₃ |
| I-293 | Q.9 | 6-CF₃, 5-OCH₃ |
| I-294 | Q.9 | 6-CF₃, 5-CF₃ |
| I-295 | Q.9 | 6-CF₃, 5-SO₂CH₃ |
| I-296 | Q.9 | 6-CF₃, 5-SCF₃ |
| I-297 | Q.9 | 6-CF₃, 5-SO₂CF₃ |
| I-298 | Q.9 | 6-CF₃, 5-CN |
| I-299 | Q.9 | 6-CF₃, 5-OCF₃ |
| I-300 | Q.10 | 3-F, 6-F |
| I-301 | Q.10 | 3-F, 6-Cl |
| I-302 | Q.10 | 3-F, 6-Br |
| I-303 | Q.10 | 3-F, 6-CH₃ |
| I-304 | Q.10 | 3-F, 6-OCH₃ |
| I-305 | Q.10 | 3-F, 6-CF₃ |
| I-306 | Q.10 | 3-F, 6-SO₂CH₃ |
| I-307 | Q.10 | 3-F, 6-SCF₃ |
| I-308 | Q.10 | 3-F, 6-SO₂CF₃ |
| I-309 | Q.10 | 3-F, 6-CN |
| I-310 | Q.10 | 3-F, 6-OCF₃ |
| I-311 | Q.10 | 3-Cl, 6-F |
| I-312 | Q.10 | 3-Cl, 6-Cl |
| I-313 | Q.10 | 3-Cl, 6-Br |
| I-314 | Q.10 | 3-Cl, 6-CH₃ |
| I-315 | Q.10 | 3-Cl, 6-OCH₃ |
| I-316 | Q.10 | 3-Cl, 6-CF₃ |
| I-317 | Q.10 | 3-Cl, 6-SO₂CH₃ |
| I-318 | Q.10 | 3-Cl, 6-SCF₃ |
| I-319 | Q.10 | 3-Cl, 6-SO₂CF₃ |
| I-320 | Q.10 | 3-Cl, 6-CN |
| I-321 | Q.10 | 3-Cl, 6-OCF₃ |
| I-322 | Q.10 | 3-Br, 6-F |
| I-323 | Q.10 | 3-Br, 6-Cl |
| I-324 | Q.10 | 3-Br, 6-Br |
| I-325 | Q.10 | 3-Br, 6-CH₃ |
| I-326 | Q.10 | 3-Br, 6-OCH₃ |
| I-327 | Q.10 | 3-Br, 6-CF₃ |
| I-328 | Q.10 | 3-Br, 6-SO₂CH₃ |
| I-329 | Q.10 | 3-Br, 6-SCF₃ |
| I-330 | Q.10 | 3-Br, 6-SO₂CF₃ |
| I-331 | Q.10 | 3-Br, 6-CN |
| I-332 | Q.10 | 3-Br, 6-OCF₃ |
| I-333 | Q.10 | 3-CH₃, 6-F |
| I-334 | Q.10 | 3-CH₃, 6-Cl |
| I-335 | Q.10 | 3-CH₃, 6-Br |
| I-336 | Q.10 | 3-CH₃, 6-CH₃ |
| I-337 | Q.10 | 3-CH₃, 6-OCH₃ |
| I-338 | Q.10 | 3-CH₃, 6-CF₃ |
| I-339 | Q.10 | 3-CH₃, 6-SO₂CH₃ |
| I-340 | Q.10 | 3-CH₃, 6-SCF₃ |
| I-341 | Q.10 | 3-CH₃, 6-SO₂CF₃ |
| I-342 | Q.10 | 3-CH₃, 6-CN |
| I-343 | Q.10 | 3-CH₃, 6-OCF₃ |
| I-344 | Q.10 | 3-OCH₃, 6-F |
| I-345 | Q.10 | 3-OCH₃, 6-Cl |
| I-346 | Q.10 | 3-OCH₃, 6-Br |
| I-347 | Q.10 | 3-OCH₃, 6-CH₃ |
| I-348 | Q.10 | 3-OCH₃, 6-OCH₃ |
| I-349 | Q.10 | 3-OCH₃, 6-CF₃ |
| I-350 | Q.10 | 3-OCH₃, 6-SO₂CH₃ |
| I-351 | Q.10 | 3-OCH₃, 6-SCF₃ |
| I-352 | Q.10 | 3-OCH₃, 6-SO₂CF₃ |
| I-353 | Q.10 | 3-OCH₃, 6-CN |
| I-354 | Q.10 | 3-OCH₃, 6-OCF₃ |
| I-355 | Q.10 | 3-CF₃, 6-F |
| I-356 | Q.10 | 3-CF₃, 6-Cl |
| I-357 | Q.10 | 3-CF₃, 6-Br |
| I-358 | Q.10 | 3-CF₃, 6-CH₃ |
| I-359 | Q.10 | 3-CF₃, 6-OCH₃ |
| I-360 | Q.10 | 3-CF₃, 6-CF₃ |
| I-361 | Q.10 | 3-CF₃, 6-SO₂CH₃ |
| I-362 | Q.10 | 3-CF₃, 6-SCF₃ |
| I-363 | Q.10 | 3-CF₃, 6-SO₂CF₃ |
| I-364 | Q.10 | 3-CF₃, 6-CN |
| I-365 | Q.10 | 3-CF₃, 6-OCF₃ |
| I-366 | Q.11 | 4-F, 6-F |
| I-367 | Q.11 | 4-F, 6-Cl |
| I-368 | Q.11 | 4-F, 6-Br |
| I-369 | Q.11 | 4-F, 6-CH₃ |
| I-370 | Q.11 | 4-F, 6-OCH₃ |
| I-371 | Q.11 | 4-F, 6-CF₃ |
| I-372 | Q.11 | 4-F, 6-SO₂CH₃ |
| I-373 | Q.11 | 4-F, 6-SCF₃ |
| I-374 | Q.11 | 4-F, 6-SO₂CF₃ |
| I-375 | Q.11 | 4-F, 6-CN |
| I-376 | Q.11 | 4-F, 6-OCF₃ |
| I-377 | Q.11 | 4-Cl, 6-F |
| I-378 | Q.11 | 4-Cl, 6-Cl |
| I-379 | Q.11 | 4-Cl, 6-Br |
| I-380 | Q.11 | 4-Cl, 6-CH₃ |
| I-381 | Q.11 | 4-Cl, 6-OCH₃ |
| I-382 | Q.11 | 4-Cl, 6-CF₃ |
| I-383 | Q.11 | 4-Cl, 6-SO₂CH₃ |
| I-384 | Q.11 | 4-Cl, 6-SCF₃ |
| I-385 | Q.11 | 4-Cl, 6-SO₂CF₃ |
| I-386 | Q.11 | 4-Cl, 6-CN |
| I-387 | Q.11 | 4-Cl, 6-OCF₃ |
| I-388 | Q.11 | 4-Br, 6-F |
| I-389 | Q.11 | 4-Br, 6-Cl |
| I-390 | Q.11 | 4-Br, 6-Br |
| I-391 | Q.11 | 4-Br, 6-CH₃ |
| I-392 | Q.11 | 4-Br, 6-OCH₃ |
| I-393 | Q.11 | 4-Br, 6-CF₃ |
| I-394 | Q.11 | 4-Br, 6-SO₂CH₃ |
| I-395 | Q.11 | 4-Br, 6-SCF₃ |
| I-396 | Q.11 | 4-Br, 6-SO₂CF₃ |
| I-397 | Q.11 | 4-Br, 6-CN |
| I-398 | Q.11 | 4-Br, 6-OCF₃ |
| I-399 | Q.11 | 4-CH₃, 6-F |
| I-400 | Q.11 | 4-CH₃, 6-Cl |
| I-401 | Q.11 | 4-CH₃, 6-Br |
| I-402 | Q.11 | 4-CH₃, 6-CH₃ |
| I-403 | Q.11 | 4-CH₃, 6-OCH₃ |
| I-404 | Q.11 | 4-CH₃, 6-CF₃ |
| I-405 | Q.11 | 4-CH₃, 6-SO₂CH₃ |
| I-406 | Q.11 | 4-CH₃, 6-SCF₃ |
| I-407 | Q.11 | 4-CH₃, 6-SO₂CF₃ |
| I-408 | Q.11 | 4-CH₃, 6-CN |
| I-409 | Q.11 | 4-CH₃, 6-OCF₃ |
| I-410 | Q.11 | 4-OCH₃, 6-F |
| I-411 | Q.11 | 4-OCH₃, 6-Cl |
| I-412 | Q.11 | 4-OCH₃, 6-Br |
| I-413 | Q.11 | 4-OCH₃, 6-CH₃ |
| I-414 | Q.11 | 4-OCH₃, 6-OCH₃ |
| I-415 | Q.11 | 4-OCH₃, 6-CF₃ |
| I-416 | Q.11 | 4-OCH₃, 6-SO₂CH₃ |
| I-417 | Q.11 | 4-OCH₃, 6-SCF₃ |
| I-418 | Q.11 | 4-OCH₃, 6-SO₂CF₃ |
| I-419 | Q.11 | 4-OCH₃, 6-CN |
| I-420 | Q.11 | 4-OCH₃, 6-OCF₃ |
| I-421 | Q.11 | 4-CF₃, 6-F |
| I-422 | Q.11 | 4-CF₃, 6-Cl |
| I-423 | Q.11 | 4-CF₃, 6-Br |
| I-424 | Q.11 | 4-CF₃, 6-CH₃ |
| I-425 | Q.11 | 4-CF₃, 6-OCH₃ |
| I-426 | Q.11 | 4-CF₃, 6-CF₃ |
| I-427 | Q.11 | 4-CF₃, 6-SO₂CH₃ |
| I-428 | Q.11 | 4-CF₃, 6-SCF₃ |
| I-429 | Q.11 | 4-CF₃, 6-SO₂CF₃ |
| I-430 | Q.11 | 4-CF₃, 6-CN |
| I-431 | Q.11 | 4-CF₃, 6-OCF₃ |
| I-432 | Q.30 | — |
| I-433 | Q.31 | 4-F |
| I-434 | Q.31 | 4-Cl |
| I-435 | Q.31 | 4-Br |
| I-436 | Q.31 | 4-CH₃ |
| I-437 | Q.31 | 4-OCH₃ |
| I-438 | Q.31 | 4-CF₃ |
| I-439 | Q.32 | 5-F |
| I-440 | Q.32 | 5-Cl |
| I-441 | Q.32 | 5-Br |
| I-442 | Q.32 | 5-CH₃ |
| I-443 | Q.32 | 5-OCH₃ |
| I-444 | Q.32 | 5-CF₃ |
| I-445 | Q.32 | 5-SO₂CH₃ |
| I-446 | Q.32 | 5-SCF₃ |
| I-447 | Q.32 | 5-SO₂CF₃ |
| I-448 | Q.32 | 5-CN |
| I-449 | Q.32 | 5-OCF₃ |
| I-450 | Q.33 | 6-F |
| I-451 | Q.33 | 6-Cl |
| I-452 | Q.33 | 6-Br |
| I-453 | Q.33 | 6-CH₃ |
| I-454 | Q.33 | 6-OCH₃ |
| I-455 | Q.33 | 6-CF₃ |
| I-456 | Q.33 | 6-SO₂CH₃ |
| I-457 | Q.33 | 6-SCF₃ |
| I-458 | Q.33 | 6-SO₂CF₃ |
| I-459 | Q.33 | 6-CN |
| I-460 | Q.33 | 6-OCF₃ |
| I-461 | Q.34 | 4-F, 6-F |
| I-462 | Q.34 | 4-F, 6-Cl |
| I-463 | Q.34 | 4-F, 6-Br |
| I-464 | Q.34 | 4-F, 6-CH₃ |
| I-465 | Q.34 | 4-F, 6-OCH₃ |
| I-466 | Q.34 | 4-F, 6-CF₃ |
| I-467 | Q.34 | 4-F, 6-SO₂CH₃ |
| I-468 | Q.34 | 4-F, 6-SCF₃ |
| I-469 | Q.34 | 4-F, 6-SO₂CF₃ |
| I-470 | Q.34 | 4-F, 6-CN |
| I-471 | Q.34 | 4-F, 6-OCF₃ |
| I-472 | Q.34 | 4-Cl, 6-F |
| I-473 | Q.34 | 4-Cl, 6-Cl |
| I-474 | Q.34 | 4-Cl, 6-Br |
| I-475 | Q.34 | 4-Cl, 6-CH₃ |
| I-476 | Q.34 | 4-Cl, 6-OCH₃ |
| I-477 | Q.34 | 4-Cl, 6-CF₃ |
| I-478 | Q.34 | 4-Cl, 6-SO₂CH₃ |
| I-479 | Q.34 | 4-Cl, 6-SCF₃ |
| I-480 | Q.34 | 4-Cl, 6-SO₂CF₃ |
| I-481 | Q.34 | 4-Cl, 6-CN |
| I-482 | Q.34 | 4-Cl, 6-OCF₃ |
| I-483 | Q.34 | 4-Br, 6-F |
| I-484 | Q.34 | 4-Br, 6-Cl |
| I-485 | Q.34 | 4-Br, 6-Br |
| I-486 | Q.34 | 4-Br, 6-CH₃ |
| I-487 | Q.34 | 4-Br, 6-OCH₃ |
| I-488 | Q.34 | 4-Br, 6-CF₃ |
| I-489 | Q.34 | 4-Br, 6-SO₂CH₃ |
| I-490 | Q.34 | 4-Br, 6-SCF₃ |
| I-491 | Q.34 | 4-Br, 6-SO₂CF₃ |
| I-492 | Q.34 | 4-Br, 6-CN |
| I-493 | Q.34 | 4-Br, 6-OCF₃ |
| I-494 | Q.34 | 4-CH₃, 6-F |
| I-495 | Q.34 | 4-CH₃, 6-Cl |
| I-496 | Q.34 | 4-CH₃, 6-Br |
| I-497 | Q.34 | 4-CH₃, 6-CH₃ |
| I-498 | Q.34 | 4-CH₃, 6-OCH₃ |
| I-499 | Q.34 | 4-CH₃, 6-CF₃ |
| I-500 | Q.34 | 4-CH₃, 6-SO₂CH₃ |
| I-501 | Q.34 | 4-CH₃, 6-SCF₃ |
| I-502 | Q.34 | 4-CH₃, 6-SO₂CF₃ |
| I-503 | Q.34 | 4-CH₃, 6-CN |
| I-504 | Q.34 | 4-CH₃, 6-OCF₃ |
| I-505 | Q.34 | 4-OCH₃, 6-F |
| I-506 | Q.34 | 4-OCH₃, 6-Cl |
| I-507 | Q.34 | 4-OCH₃, 6-Br |
| I-508 | Q.34 | 4-OCH₃, 6-CH₃ |
| I-509 | Q.34 | 4-OCH₃, 6-OCH₃ |
| I-510 | Q.34 | 4-OCH₃, 6-CF₃ |
| I-511 | Q.34 | 4-OCH₃, 6-SO₂CH₃ |
| I-512 | Q.34 | 4-OCH₃, 6-SCF₃ |
| I-513 | Q.34 | 4-OCH₃, 6-SO₂CF₃ |
| I-514 | Q.34 | 4-OCH₃, 6-CN |
| I-515 | Q.34 | 4-OCH₃, 6-OCF₃ |
| I-516 | Q.34 | 4-CF₃, 6-F |
| I-517 | Q.34 | 4-CF₃, 6-Cl |
| I-518 | Q.34 | 4-CF₃, 6-Br |
| I-519 | Q.34 | 4-CF₃, 6-CH₃ |
| I-520 | Q.34 | 4-CF₃, 6-OCH₃ |
| I-521 | Q.34 | 4-CF₃, 6-CF₃ |
| I-522 | Q.34 | 4-CF₃, 6-SO₂CH₃ |
| I-523 | Q.34 | 4-CF₃, 6-SCF₃ |
| I-524 | Q.34 | 4-CF₃, 6-SO₂CF₃ |
| I-525 | Q.34 | 4-CF₃, 6-CN |
| I-526 | Q.34 | 4-CF₃, 6-OCF₃ |
| I-527 | Q.35 | 4-F, 5-F |
| I-528 | Q.35 | 4-F, 5-Cl |
| I-529 | Q.35 | 4-F, 5-Br |
| I-530 | Q.35 | 4-F, 5-CH₃ |
| I-531 | Q.35 | 4-F, 5-OCH₃ |
| I-532 | Q.35 | 4-F, 5-CF₃ |
| I-533 | Q.35 | 4-F, 5-SO₂CH₃ |
| I-534 | Q.35 | 4-F, 5-SCF₃ |
| I-535 | Q.35 | 4-F, 5-SO₂CF₃ |
| I-536 | Q.35 | 4-F, 5-CN |
| I-537 | Q.35 | 4-F, 5-OCF₃ |
| I-538 | Q.35 | 4-Cl, 5-F |
| I-539 | Q.35 | 4-Cl, 5-Cl |
| I-540 | Q.35 | 4-Cl, 5-Br |
| I-541 | Q.35 | 4-Cl, 5-CH₃ |
| I-542 | Q.35 | 4-Cl, 5-OCH₃ |
| I-543 | Q.35 | 4-Cl, 5-CF₃ |
| I-544 | Q.35 | 4-Cl, 5-SO₂CH₃ |
| I-545 | Q.35 | 4-Cl, 5-SCF₃ |
| I-546 | Q.35 | 4-Cl, 5-SO₂CF₃ |
| I-547 | Q.35 | 4-Cl, 5-CN |
| I-548 | Q.35 | 4-Cl, 5-OCF₃ |
| I-549 | Q.35 | 4-Br, 5-F |
| I-550 | Q.35 | 4-Br, 5-Cl |
| I-551 | Q.35 | 4-Br, 5-Br |
| I-552 | Q.35 | 4-Br, 5-CH₃ |
| I-553 | Q.35 | 4-Br, 5-OCH₃ |
| I-554 | Q.35 | 4-Br, 5-CF₃ |
| I-555 | Q.35 | 4-Br, 5-SO₂CH₃ |
| I-556 | Q.35 | 4-Br, 5-SCF₃ |
| I-557 | Q.35 | 4-Br, 5-SO₂CF₃ |
| I-558 | Q.35 | 4-Br, 5-CN |
| I-559 | Q.35 | 4-Br, 5-OCF₃ |
| I-560 | Q.35 | 4-CH₃, 5-F |
| I-561 | Q.35 | 4-CH₃, 5-Cl |
| I-562 | Q.35 | 4-CH₃, 5-Br |
| I-563 | Q.35 | 4-CH₃, 5-CH₃ |
| I-564 | Q.35 | 4-CH₃, 5-OCH₃ |
| I-565 | Q.35 | 4-CH₃, 5-CF₃ |
| I-566 | Q.35 | 4-CH₃, 5-SO₂CH₃ |
| I-567 | Q.35 | 4-CH₃, 5-SCF₃ |
| I-568 | Q.35 | 4-CH₃, 5-SO₂CF₃ |
| I-569 | Q.35 | 4-CH₃, 5-CN |
| I-570 | Q.35 | 4-CH₃, 5-OCF₃ |
| I-571 | Q.35 | 4-OCH₃, 5-F |
| I-572 | Q.35 | 4-OCH₃, 5-Cl |
| I-573 | Q.35 | 4-OCH₃, 5-Br |
| I-574 | Q.35 | 4-OCH₃, 5-CH₃ |
| I-575 | Q.35 | 4-OCH₃, 5-OCH₃ |
| I-576 | Q.35 | 4-OCH₃, 5-CF₃ |
| I-577 | Q.35 | 4-OCH₃, 5-SO₂CH₃ |
| I-578 | Q.35 | 4-OCH₃, 5-SCF₃ |
| I-579 | Q.35 | 4-OCH₃, 5-SO₂CF₃ |
| I-580 | Q.35 | 4-OCH₃, 5-CN |
| I-581 | Q.35 | 4-OCH₃, 5-OCF₃ |
| I-582 | Q.35 | 4-CF₃, 5-F |
| I-583 | Q.35 | 4-CF₃, 5-Cl |
| I-584 | Q.35 | 4-CF₃, 5-Br |
| I-585 | Q.35 | 4-CF₃, 5-CH₃ |
| I-586 | Q.35 | 4-CF₃, 5-OCH₃ |
| I-587 | Q.35 | 4-CF₃, 5-CF₃ |
| I-588 | Q.35 | 4-CF₃, 5-SO₂CH₃ |
| I-589 | Q.35 | 4-CF₃, 5-SCF₃ |
| I-590 | Q.35 | 4-CF₃, 5-SO₂CF₃ |
| I-591 | Q.35 | 4-CF₃, 5-CN |
| I-592 | Q.35 | 4-CF₃, 5-OCF₃ |
| I-593 | Q.36 | 5-F, 6-F |
| I-594 | Q.36 | 5-F, 6-Cl |
| I-595 | Q.36 | 5-F, 6-Br |
| I-596 | Q.36 | 5-F, 6-CH₃ |
| I-597 | Q.36 | 5-F, 6-OCH₃ |
| I-598 | Q.36 | 5-F, 6-CF₃ |
| I-599 | Q.36 | 5-F, 6-SO₂CH₃ |
| I-600 | Q.36 | 5-F, 6-SCF₃ |
| I-601 | Q.36 | 5-F, 6-SO₂CF₃ |
| I-602 | Q.36 | 5-F, 6-CN |
| I-603 | Q.36 | 5-F, 6-OCF₃ |
| I-604 | Q.36 | 5-Cl, 6-F |
| I-605 | Q.36 | 5-Cl, 6-Cl |
| I-606 | Q.36 | 5-Cl, 6-Br |
| I-607 | Q.36 | 5-Cl, 6-CH₃ |
| I-608 | Q.36 | 5-Cl, 6-OCH₃ |
| I-609 | Q.36 | 5-Cl, 6-CF₃ |
| I-610 | Q.36 | 5-Cl, 6-SO₂CH₃ |
| I-611 | Q.36 | 5-Cl, 6-SCF₃ |
| I-612 | Q.36 | 5-Cl, 6-SO₂CF₃ |
| I-613 | Q.36 | 5-Cl, 6-CN |
| I-614 | Q.36 | 5-Cl, 6-OCF₃ |
| I-615 | Q.36 | 5-Br, 6-F |
| I-616 | Q.36 | 5-Br, 6-Cl |
| I-617 | Q.36 | 5-Br, 6-Br |
| I-618 | Q.36 | 5-Br, 6-CH₃ |
| I-619 | Q.36 | 5-Br, 6-OCH₃ |
| I-620 | Q.36 | 5-Br, 6-CF₃ |
| I-621 | Q.36 | 5-Br, 6-SO₂CH₃ |
| I-622 | Q.36 | 5-Br, 6-SCF₃ |
| I-623 | Q.36 | 5-Br, 6-SO₂CF₃ |
| I-624 | Q.36 | 5-Br, 6-CN |
| I-625 | Q.36 | 5-Br, 6-OCF₃ |
| I-626 | Q.36 | 5-CH₃, 6-F |
| I-627 | Q.36 | 5-CH₃, 6-Cl |
| I-628 | Q.36 | 5-CH₃, 6-Br |
| I-629 | Q.36 | 5-CH₃, 6-CH₃ |
| I-630 | Q.36 | 5-CH₃, 6-OCH₃ |
| I-631 | Q.36 | 5-CH₃, 6-CF₃ |
| I-632 | Q.36 | 5-CH₃, 6-SO₂CH₃ |
| I-633 | Q.36 | 5-CH₃, 6-SCF₃ |
| I-634 | Q.36 | 5-CH₃, 6-SO₂CF₃ |
| I-635 | Q.36 | 5-CH₃, 6-CN |
| I-636 | Q.36 | 5-CH₃, 6-OCF₃ |
| I-637 | Q.36 | 5-OCH₃, 6-F |
| I-638 | Q.36 | 5-OCH₃, 6-Cl |
| I-639 | Q.36 | 5-OCH₃, 6-Br |
| I-640 | Q.36 | 5-OCH₃, 6-CH₃ |
| I-641 | Q.36 | 5-OCH₃, 6-OCH₃ |
| I-642 | Q.36 | 5-OCH₃, 6-CF₃ |
| I-643 | Q.36 | 5-OCH₃, 6-SO₂CH₃ |
| I-644 | Q.36 | 5-OCH₃, 6-SCF₃ |
| I-645 | Q.36 | 5-OCH₃, 6-SO₂CF₃ |
| I-646 | Q.36 | 5-OCH₃, 6-CN |
| I-647 | Q.36 | 5-OCH₃, 6-OCF₃ |
| I-648 | Q.36 | 5-CF₃, 6-F |
| I-649 | Q.36 | 5-CF₃, 6-Cl |
| I-650 | Q.36 | 5-CF₃, 6-Br |
| I-651 | Q.36 | 5-CF₃, 6-CH₃ |
| I-652 | Q.36 | 5-CF₃, 6-OCH₃ |
| I-653 | Q.36 | 5-CF₃, 6-CF₃ |
| I-654 | Q.36 | 5-CF₃, 6-SO₂CH₃ |
| I-655 | Q.36 | 5-CF₃, 6-SCF₃ |
| I-656 | Q.36 | 5-CF₃, 6-SO₂CF₃ |
| I-657 | Q.36 | 5-CF₃, 6-CN |
| I-658 | Q.36 | 5-CF₃, 6-OCF₃ |
| I-659 | Q.44 | — |
| I-660 | Q.45 | 4-F |
| I-661 | Q.45 | 4-Cl |
| I-662 | Q.45 | 4-Br |
| I-663 | Q.45 | 4-CH₃ |
| I-664 | Q.45 | 4-OCH₃ |
| I-665 | Q.45 | 4-CF₃ |
| I-666 | Q.46 | 5-F |
| I-667 | Q.46 | 5-Cl |
| I-668 | Q.46 | 5-Br |
| I-669 | Q.46 | 5-CH₃ |
| I-670 | Q.46 | 5-OCH₃ |
| I-671 | Q.46 | 5-CF₃ |
| I-672 | Q.46 | 5-SO₂CH₃ |
| I-673 | Q.46 | 5-SCF₃ |
| I-674 | Q.46 | 5-SO₂CF₃ |
| I-675 | Q.46 | 5-CN |
| I-676 | Q.46 | 5-OCF₃ |
| I-677 | Q.47 | 6-F |
| I-678 | Q.47 | 6-Cl |
| I-679 | Q.47 | 6-Br |
| I-680 | Q.47 | 6-CH₃ |
| I-681 | Q.47 | 6-OCH₃ |
| I-682 | Q.47 | 6-CF₃ |
| I-683 | Q.47 | 6-SO₂CH₃ |
| I-684 | Q.47 | 6-SCF₃ |
| I-685 | Q.47 | 6-SO₂CF₃ |
| I-686 | Q.47 | 6-CN |
| I-687 | Q.47 | 6-OCF₃ |
| I-688 | Q.48 | 4-F, 5-F |
| I-689 | Q.48 | 4-F, 5-Cl |
| I-690 | Q.48 | 4-F, 5-Br |
| I-691 | Q.48 | 4-F, 5-CH₃ |
| I-692 | Q.48 | 4-F, 5-OCH₃ |
| I-693 | Q.48 | 4-F, 5-CF₃ |
| I-694 | Q.48 | 4-F, 5-SO₂CH₃ |
| I-695 | Q.48 | 4-F, 5-SCF₃ |
| I-696 | Q.48 | 4-F, 5-SO₂CF₃ |
| I-697 | Q.48 | 4-F, 5-CN |
| I-698 | Q.48 | 4-F, 5-OCF₃ |
| I-699 | Q.48 | 4-Cl, 5-F |
| I-700 | Q.48 | 4-Cl, 5-Cl |
| I-701 | Q.48 | 4-Cl, 5-Br |
| I-702 | Q.48 | 4-Cl, 5-CH₃ |
| I-703 | Q.48 | 4-Cl, 5-OCH₃ |
| I-704 | Q.48 | 4-Cl, 5-CF₃ |
| I-705 | Q.48 | 4-Cl, 5-SO₂CH₃ |
| I-706 | Q.48 | 4-Cl, 5-SCF₃ |
| I-707 | Q.48 | 4-Cl, 5-SO₂CF₃ |
| I-708 | Q.48 | 4-Cl, 5-CN |
| I-709 | Q.48 | 4-Cl, 5-OCF₃ |
| I-710 | Q.48 | 4-Br, 5-F |
| I-711 | Q.48 | 4-Br, 5-Cl |
| I-712 | Q.48 | 4-Br, 5-Br |
| I-713 | Q.48 | 4-Br, 5-CH₃ |
| I-714 | Q.48 | 4-Br, 5-OCH₃ |
| I-715 | Q.48 | 4-Br, 5-CF₃ |
| I-716 | Q.48 | 4-Br, 5-SO₂CH₃ |
| I-717 | Q.48 | 4-Br, 5-SCF₃ |
| I-718 | Q.48 | 4-Br, 5-SO₂CF₃ |
| I-719 | Q.48 | 4-Br, 5-CN |
| I-720 | Q.48 | 4-Br, 5-OCF₃ |
| I-721 | Q.48 | 4-CH₃, 5-F |
| I-722 | Q.48 | 4-CH₃, 5-Cl |
| I-723 | Q.48 | 4-CH₃, 5-Br |
| I-724 | Q.48 | 4-CH₃, 5-CH₃ |
| I-725 | Q.48 | 4-CH₃, 5-OCH₃ |
| I-726 | Q.48 | 4-CH₃, 5-CF₃ |
| I-727 | Q.48 | 4-CH₃, 5-SO₂CH₃ |
| I-728 | Q.48 | 4-CH₃, 5-SCF₃ |
| I-729 | Q.48 | 4-CH₃, 5-SO₂CF₃ |
| I-730 | Q.48 | 4-CH₃, 5-CN |
| I-731 | Q.48 | 4-CH₃, 5-OCF₃ |
| I-732 | Q.48 | 4-OCH₃, 5-F |
| I-733 | Q.48 | 4-OCH₃, 5-Cl |
| I-734 | Q.48 | 4-OCH₃, 5-Br |
| I-735 | Q.48 | 4-OCH₃, 5-CH₃ |
| I-736 | Q.48 | 4-OCH₃, 5-OCH₃ |
| I-737 | Q.48 | 4-OCH₃, 5-CF₃ |
| I-738 | Q.48 | 4-OCH₃, 5-SO₂CH₃ |
| I-739 | Q.48 | 4-OCH₃, 5-SCF₃ |
| I-740 | Q.48 | 4-OCH₃, 5-SO₂CF₃ |
| I-741 | Q.48 | 4-OCH₃, 5-CN |
| I-742 | Q.48 | 4-OCH₃, 5-OCF₃ |
| I-743 | Q.48 | 4-CF₃, 5-F |
| I-744 | Q.48 | 4-CF₃, 5-Cl |
| I-745 | Q.48 | 4-CF₃, 5-Br |
| I-746 | Q.48 | 4-CF₃, 5-CH₃ |
| I-747 | Q.48 | 4-CF₃, 5-OCH₃ |
| I-748 | Q.48 | 4-CF₃, 5-CF₃ |
| I-749 | Q.48 | 4-CF₃, 5-SO₂CH₃ |
| I-750 | Q.48 | 4-CF₃, 5-SCF₃ |
| I-751 | Q.48 | 4-CF₃, 5-SO₂CF₃ |
| I-752 | Q.48 | 4-CF₃, 5-CN |
| I-753 | Q.48 | 4-CF₃, 5-OCF₃ |
| I-754 | Q.49 | 4-F, 6-F |
| I-755 | Q.49 | 4-F, 6-Cl |
| I-756 | Q.49 | 4-F, 6-Br |
| I-757 | Q.49 | 4-F, 6-CH₃ |
| I-758 | Q.49 | 4-F, 6-OCH₃ |
| I-759 | Q.49 | 4-F, 6-CF₃ |
| I-760 | Q.49 | 4-F, 6-SO₂CH₃ |
| I-761 | Q.49 | 4-F, 6-SCF₃ |
| I-762 | Q.49 | 4-F, 6-SO₂CF₃ |
| I-763 | Q.49 | 4-F, 6-CN |
| I-764 | Q.49 | 4-F, 6-OCF₃ |
| I-765 | Q.49 | 4-Cl, 6-F |
| I-766 | Q.49 | 4-Cl, 6-Cl |
| I-767 | Q.49 | 4-Cl, 6-Br |
| I-768 | Q.49 | 4-Cl, 6-CH₃ |
| I-769 | Q.49 | 4-Cl, 6-OCH₃ |
| I-770 | Q.49 | 4-Cl, 6-CF₃ |
| I-771 | Q.49 | 4-Cl, 6-SO₂CH₃ |
| I-772 | Q.49 | 4-Cl, 6-SCF₃ |
| I-773 | Q.49 | 4-Cl, 6-SO₂CF₃ |
| I-774 | Q.49 | 4-Cl, 6-CN |
| I-775 | Q.49 | 4-Cl, 6-OCF₃ |
| I-776 | Q.49 | 4-Br, 6-F |
| I-777 | Q.49 | 4-Br, 6-Cl |
| I-778 | Q.49 | 4-Br, 6-Br |
| I-779 | Q.49 | 4-Br, 6-CH₃ |
| I-780 | Q.49 | 4-Br, 6-OCH₃ |
| I-781 | Q.49 | 4-Br, 6-CF₃ |
| I-782 | Q.49 | 4-Br, 6-SO₂CH₃ |
| I-783 | Q.49 | 4-Br, 6-SCF₃ |
| I-784 | Q.49 | 4-Br, 6-SO₂CF₃ |
| I-785 | Q.49 | 4-Br, 6-CN |
| I-786 | Q.49 | 4-Br, 6-OCF₃ |
| I-787 | Q.49 | 4-CH₃, 6-F |
| I-788 | Q.49 | 4-CH₃, 6-Cl |
| I-789 | Q.49 | 4-CH₃, 6-Br |
| I-790 | Q.49 | 4-CH₃, 6-CH₃ |
| I-791 | Q.49 | 4-CH₃, 6-OCH₃ |
| I-792 | Q.49 | 4-CH₃, 6-CF₃ |
| I-793 | Q.49 | 4-CH₃, 6-SO₂CH₃ |
| I-794 | Q.49 | 4-CH₃, 6-SCF₃ |
| I-795 | Q.49 | 4-CH₃, 6-SO₂CF₃ |
| I-796 | Q.49 | 4-CH₃, 6-CN |
| I-797 | Q.49 | 4-CH₃, 6-OCF₃ |
| I-798 | Q.49 | 4-OCH₃, 6-F |
| I-799 | Q.49 | 4-OCH₃, 6-Cl |
| I-800 | Q.49 | 4-OCH₃, 6-Br |
| I-801 | Q.49 | 4-OCH₃, 6-CH₃ |
| I-802 | Q.49 | 4-OCH₃, 6-OCH₃ |
| I-803 | Q.49 | 4-OCH₃, 6-CF₃ |
| I-804 | Q.49 | 4-OCH₃, 6-SO₂CH₃ |
| I-805 | Q.49 | 4-OCH₃, 6-SCF₃ |
| I-806 | Q.49 | 4-OCH₃, 6-SO₂CF₃ |
| I-807 | Q.49 | 4-OCH₃, 6-CN |
| I-808 | Q.49 | 4-OCH₃, 6-OCF₃ |
| I-809 | Q.49 | 4-CF₃, 6-F |
| I-810 | Q.49 | 4-CF₃, 6-Cl |
| I-811 | Q.49 | 4-CF₃, 6-Br |
| I-812 | Q.49 | 4-CF₃, 6-CH₃ |
| I-813 | Q.49 | 4-CF₃, 6-OCH₃ |
| I-814 | Q.49 | 4-CF₃, 6-CF₃ |
| I-815 | Q.49 | 4-CF₃, 6-SO₂CH₃ |
| I-816 | Q.49 | 4-CF₃, 6-SCF₃ |
| I-817 | Q.49 | 4-CF₃, 6-SO₂CF₃ |
| I-818 | Q.49 | 4-CF₃, 6-CN |
| I-819 | Q.49 | 4-CF₃, 6-OCF₃ |
| I-820 | Q.50 | — |
| I-821 | Q.51 | 5-F |
| I-822 | Q.51 | 5-Cl |
| I-823 | Q.51 | 5-Br |
| I-824 | Q.51 | 5-CH₃ |
| I-825 | Q.51 | 5-OCH₃ |
| I-826 | Q.51 | 5-CF₃ |
| I-827 | Q.51 | 5-SO₂CH₃ |
| I-828 | Q.51 | 5-SCF₃ |
| I-829 | Q.51 | 5-SO₂CF₃ |
| I-830 | Q.51 | 5-CN |
| I-831 | Q.51 | 5-OCF₃ |
| I-832 | Q.52 | 5-F |
| I-833 | Q.52 | 5-Cl |
| I-834 | Q.52 | 5-Br |
| I-835 | Q.52 | 5-CH₃ |
| I-836 | Q.52 | 5-OCH₃ |
| I-837 | Q.52 | 5-CF₃ |
| I-838 | Q.53 | 5-F |
| I-839 | Q.53 | 5-Cl |
| I-840 | Q.53 | 5-Br |
| I-841 | Q.53 | 5-CH₃ |
| I-842 | Q.53 | 5-OCH₃ |
| I-843 | Q.53 | 5-CF₃ |
| I-844 | Q.53 | 5-SO₂CH₃ |
| I-845 | Q.53 | 5-SCF₃ |
| I-846 | Q.53 | 5-SO₂CF₃ |
| I-847 | Q.53 | 5-CN |
| I-848 | Q.53 | 5-OCF₃ |
| I-849 | Q.54 | 6-F, 2-F |
| I-850 | Q.54 | 6-F, 2-Cl |
| I-851 | Q.54 | 6-F, 2-Br |
| I-852 | Q.54 | 6-F, 2-CH₃ |
| I-853 | Q.54 | 6-F, 2-OCH₃ |
| I-854 | Q.54 | 6-F, 2-CF₃ |
| I-855 | Q.54 | 6-F, 2-SO₂CH₃ |
| I-856 | Q.54 | 6-F, 2-SCF₃ |
| I-857 | Q.54 | 6-F, 2-SO₂CF₃ |
| I-858 | Q.54 | 6-F, 2-CN |
| I-859 | Q.54 | 6-F, 2-OCF₃ |
| I-860 | Q.54 | 6-Cl, 2-F |
| I-861 | Q.54 | 6-Cl, 2-Cl |
| I-862 | Q.54 | 6-Cl, 2-Br |
| I-863 | Q.54 | 6-Cl, 2-CH₃ |
| I-864 | Q.54 | 6-Cl, 2-OCH₃ |
| I-865 | Q.54 | 6-Cl, 2-CF₃ |
| I-866 | Q.54 | 6-Cl, 2-SO₂CH₃ |
| I-867 | Q.54 | 6-Cl, 2-SCF₃ |
| I-868 | Q.54 | 6-Cl, 2-SO₂CF₃ |
| I-869 | Q.54 | 6-Cl, 2-CN |
| I-870 | Q.54 | 6-Cl, 2-OCF₃ |
| I-871 | Q.54 | 6-Br, 2-F |
| I-872 | Q.54 | 6-Br, 2-Cl |
| I-873 | Q.54 | 6-Br, 2-Br |
| I-874 | Q.54 | 6-Br, 2-CH₃ |
| I-875 | Q.54 | 6-Br, 2-OCH₃ |
| I-876 | Q.54 | 6-Br, 2-CF₃ |
| I-877 | Q.54 | 6-Br, 2-SO₂CH₃ |
| I-878 | Q.54 | 6-Br, 2-SCF₃ |
| I-879 | Q.54 | 6-Br, 2-SO₂CF₃ |
| I-880 | Q.54 | 6-Br, 2-CN |
| I-881 | Q.54 | 6-Br, 2-OCF₃ |
| I-882 | Q.54 | 6-CH₃, 2-F |
| I-883 | Q.54 | 6-CH₃, 2-Cl |
| I-884 | Q.54 | 6-CH₃, 2-Br |
| I-885 | Q.54 | 6-CH₃, 2-CH₃ |
| I-886 | Q.54 | 6-CH₃, 2-OCH₃ |
| I-887 | Q.54 | 6-CH₃, 2-CF₃ |
| I-888 | Q.54 | 6-CH₃, 2-SO₂CH₃ |
| I-889 | Q.54 | 6-CH₃, 2-SCF₃ |
| I-890 | Q.54 | 6-CH₃, 2-SO₂CF₃ |
| I-891 | Q.54 | 6-CH₃, 2-CN |
| I-892 | Q.54 | 6-CH₃, 2-OCF₃ |
| I-893 | Q.54 | 6-OCH₃, 2-F |
| I-894 | Q.54 | 6-OCH₃, 2-Cl |
| I-895 | Q.54 | 6-OCH₃, 2-Br |
| I-896 | Q.54 | 6-OCH₃, 2-CH₃ |
| I-897 | Q.54 | 6-OCH₃, 2-OCH₃ |
| I-898 | Q.54 | 6-OCH₃, 2-CF₃ |
| I-899 | Q.54 | 6-OCH₃, 2-SO₂CH₃ |
| I-900 | Q.54 | 6-OCH₃, 2-SCF₃ |
| I-901 | Q.54 | 6-OCH₃, 2-SO₂CF₃ |
| I-902 | Q.54 | 6-OCH₃, 2-CN |
| I-903 | Q.54 | 6-OCH₃, 2-OCF₃ |
| I-904 | Q.54 | 6-CF₃, 2-F |
| I-905 | Q.54 | 6-CF₃, 2-Cl |
| I-906 | Q.54 | 6-CF₃, 2-Br |
| I-907 | Q.54 | 6-CF₃, 2-CH₃ |
| I-908 | Q.54 | 6-CF₃, 2-OCH₃ |
| I-909 | Q.54 | 6-CF₃, 2-CF₃ |
| I-910 | Q.54 | 6-CF₃, 2-SO₂CH₃ |
| I-911 | Q.54 | 6-CF₃, 2-SCF₃ |
| I-912 | Q.54 | 6-CF₃, 2-SO₂CF₃ |
| I-913 | Q.54 | 6-CF₃, 2-CN |
| I-914 | Q.54 | 6-CF₃, 2-OCF₃ |
| I-915 | Q.55 | 5-F, 6-F |
| I-916 | Q.55 | 5-F, 6-Cl |
| I-917 | Q.55 | 5-F, 6-Br |
| I-918 | Q.55 | 5-F, 6-CH₃ |
| I-919 | Q.55 | 5-F, 6-OCH₃ |
| I-920 | Q.55 | 5-F, 6-CF₃ |
| I-921 | Q.55 | 5-F, 6-SO₂CH₃ |
| I-922 | Q.55 | 5-F, 6-SCF₃ |
| I-923 | Q.55 | 5-F, 6-SO₂CF₃ |
| I-924 | Q.55 | 5-F, 6-CN |
| I-925 | Q.55 | 5-F, 6-OCF₃ |
| I-926 | Q.55 | 5-Cl, 6-F |
| I-927 | Q.55 | 5-Cl, 6-Cl |
| I-928 | Q.55 | 5-Cl, 6-Br |
| I-929 | Q.55 | 5-Cl, 6-CH₃ |
| I-930 | Q.55 | 5-Cl, 6-OCH₃ |
| I-931 | Q.55 | 5-Cl, 6-CF₃ |
| I-932 | Q.55 | 5-Cl, 6-SO₂CH₃ |
| I-933 | Q.55 | 5-Cl, 6-SCF₃ |
| I-934 | Q.55 | 5-Cl, 6-SO₂CF₃ |
| I-935 | Q.55 | 5-Cl, 6-CN |
| I-936 | Q.55 | 5-Cl, 6-OCF₃ |
| I-937 | Q.55 | 5-Br, 6-F |
| I-938 | Q.55 | 5-Br, 6-Cl |
| I-939 | Q.55 | 5-Br, 6-Br |
| I-940 | Q.55 | 5-Br, 6-CH₃ |
| I-941 | Q.55 | 5-Br, 6-OCH₃ |
| I-942 | Q.55 | 5-Br, 6-CF₃ |
| I-943 | Q.55 | 5-Br, 6-SO₂CH₃ |
| I-944 | Q.55 | 5-Br, 6-SCF₃ |
| I-945 | Q.55 | 5-Br, 6-SO₂CF₃ |
| I-946 | Q.55 | 5-Br, 6-CN |
| I-947 | Q.55 | 5-Br, 6-OCF₃ |
| I-948 | Q.55 | 5-CH₃, 6-F |
| I-949 | Q.55 | 5-CH₃, 6-Cl |
| I-950 | Q.55 | 5-CH₃, 6-Br |
| I-951 | Q.55 | 5-CH₃, 6-CH₃ |
| I-952 | Q.55 | 5-CH₃, 6-OCH₃ |
| I-953 | Q.55 | 5-CH₃, 6-CF₃ |
| I-954 | Q.55 | 5-CH₃, 6-SO₂CH₃ |
| I-955 | Q.55 | 5-CH₃, 6-SCF₃ |
| I-956 | Q.55 | 5-CH₃, 6-SO₂CF₃ |
| I-957 | Q.55 | 5-CH₃, 6-CN |
| I-958 | Q.55 | 5-CH₃, 6-OCF₃ |
| I-959 | Q.55 | 5-OCH₃, 6-F |
| I-960 | Q.55 | 5-OCH₃, 6-Cl |
| I-961 | Q.55 | 5-OCH₃, 6-Br |
| I-962 | Q.55 | 5-OCH₃, 6-CH₃ |
| I-963 | Q.55 | 5-OCH₃, 6-OCH₃ |
| I-964 | Q.55 | 5-OCH₃, 6-CF₃ |
| I-965 | Q.55 | 5-OCH₃, 6-SO₂CH₃ |
| I-966 | Q.55 | 5-OCH₃, 6-SCF₃ |
| I-967 | Q.55 | 5-OCH₃, 6-SO₂CF₃ |
| I-968 | Q.55 | 5-OCH₃, 6-CN |
| I-969 | Q.55 | 5-OCH₃, 6-OCF₃ |
| I-970 | Q.55 | 5-CF₃, 6-F |
| I-971 | Q.55 | 5-CF₃, 6-Cl |
| I-972 | Q.55 | 5-CF₃, 6-Br |
| I-973 | Q.55 | 5-CF₃, 6-CH₃ |
| I-974 | Q.55 | 5-CF₃, 6-OCH₃ |
| I-975 | Q.55 | 5-CF₃, 6-CF₃ |
| I-976 | Q.55 | 5-CF₃, 6-SO₂CH₃ |
| I-977 | Q.55 | 5-CF₃, 6-SCF₃ |
| I-978 | Q.55 | 5-CF₃, 6-SO₂CF₃ |
| I-979 | Q.55 | 5-CF₃, 6-CN |
| I-980 | Q.55 | 5-CF₃, 6-OCF₃ |
| I-981 | Q.56 | 5-F, 2-F |
| I-982 | Q.56 | 5-F, 2-Cl |
| I-983 | Q.56 | 5-F, 2-Br |
| I-984 | Q.56 | 5-F, 2-CH₃ |
| I-985 | Q.56 | 5-F, 2-OCH₃ |
| I-986 | Q.56 | 5-F, 2-CF₃ |
| I-987 | Q.56 | 5-F, 2-SO₂CH₃ |
| I-988 | Q.56 | 5-F, 2-SCF₃ |
| I-989 | Q.56 | 5-F, 2-SO₂CF₃ |
| I-990 | Q.56 | 5-F, 2-CN |
| I-991 | Q.56 | 5-F, 2-OCF₃ |
| I-992 | Q.56 | 5-Cl, 2-F |
| I-993 | Q.56 | 5-Cl, 2-Cl |
| I-994 | Q.56 | 5-Cl, 2-Br |
| I-995 | Q.56 | 5-Cl, 2-CH₃ |
| I-996 | Q.56 | 5-Cl, 2-OCH₃ |
| I-997 | Q.56 | 5-Cl, 2-CF₃ |
| I-998 | Q.56 | 5-Cl, 2-SO₂CH₃ |
| I-999 | Q.56 | 5-Cl, 2-SCF₃ |
| I-1000 | Q.56 | 5-Cl, 2-SO₂CF₃ |
| I-1001 | Q.56 | 5-Cl, 2-CN |
| I-1002 | Q.56 | 5-Cl, 2-OCF₃ |
| I-1003 | Q.56 | 5-Br, 2-F |
| I-1004 | Q.56 | 5-Br, 2-Cl |
| I-1005 | Q.56 | 5-Br, 2-Br |
| I-1006 | Q.56 | 5-Br, 2-CH₃ |
| I-1007 | Q.56 | 5-Br, 2-OCH₃ |
| I-1008 | Q.56 | 5-Br, 2-CF₃ |
| I-1009 | Q.56 | 5-Br, 2-SO₂CH₃ |
| I-1010 | Q.56 | 5-Br, 2-SCF₃ |
| I-1011 | Q.56 | 5-Br, 2-SO₂CF₃ |
| I-1012 | Q.56 | 5-Br, 2-CN |
| I-1013 | Q.56 | 5-Br, 2-OCF₃ |
| I-1014 | Q.56 | 5-CH₃, 2-F |
| I-1015 | Q.56 | 5-CH₃, 2-Cl |
| I-1016 | Q.56 | 5-CH₃, 2-Br |
| I-1017 | Q.56 | 5-CH₃, 2-CH₃ |
| I-1018 | Q.56 | 5-CH₃, 2-OCH₃ |
| I-1019 | Q.56 | 5-CH₃, 2-CF₃ |
| I-1020 | Q.56 | 5-CH₃, 2-SO₂CH₃ |
| I-1021 | Q.56 | 5-CH₃, 2-SCF₃ |
| I-1022 | Q.56 | 5-CH₃, 2-SO₂CF₃ |
| I-1023 | Q.56 | 5-CH₃, 2-CN |
| I-1024 | Q.56 | 5-CH₃, 2-OCF₃ |
| I-1025 | Q.56 | 5-OCH₃, 2-F |
| I-1026 | Q.56 | 5-OCH₃, 2-Cl |
| I-1027 | Q.56 | 5-OCH₃, 2-Br |
| I-1028 | Q.56 | 5-OCH₃, 2-CH₃ |
| I-1029 | Q.56 | 5-OCH₃, 2-OCH₃ |
| I-1030 | Q.56 | 5-OCH₃, 2-CF₃ |
| I-1031 | Q.56 | 5-OCH₃, 2-SO₂CH₃ |
| I-1032 | Q.56 | 5-OCH₃, 2-SCF₃ |
| I-1033 | Q.56 | 5-OCH₃, 2-SO₂CF₃ |
| I-1034 | Q.56 | 5-OCH₃, 2-CN |
| I-1035 | Q.56 | 5-OCH₃, 2-OCF₃ |
| I-1036 | Q.56 | 5-CF₃, 2-F |
| I-1037 | Q.56 | 5-CF₃, 2-Cl |
| I-1038 | Q.56 | 5-CF₃, 2-Br |
| I-1039 | Q.56 | 5-CF₃, 2-CH₃ |
| I-1040 | Q.56 | 5-CF₃, 2-OCH₃ |
| I-1041 | Q.56 | 5-CF₃, 2-CF₃ |
| I-1042 | Q.56 | 5-CF₃, 2-CO₂CH₃ |
| I-1043 | Q.56 | 5-CF₃, 2-SCF₃ |
| I-1044 | Q.56 | 5-CF₃, 2-SO₂CF₃ |
| I-1045 | Q.56 | 5-CF₃, 2-CN |
| I-1046 | Q.56 | 5-CF₃, 2-OCF₃ |
| I-1047 | Q.57 | — |
| I-1048 | Q.58 | 3-F |
| I-1049 | Q.58 | 3-Cl |
| I-1050 | Q.58 | 3-Br |
| I-1051 | Q.58 | 3-CH₃ |
| I-1052 | Q.58 | 3-OCH₃ |
| I-1053 | Q.58 | 3-CF₃ |
| I-1054 | Q.59 | 5-F |
| I-1055 | Q.59 | 5-Cl |
| I-1056 | Q.59 | 5-Br |
| I-1057 | Q.59 | 5-CH₃ |
| I-1058 | Q.59 | 5-OCH₃ |
| I-1059 | Q.59 | 5-CF₃ |
| I-1060 | Q.59 | 5-SO₂CH₃ |
| I-1061 | Q.59 | 5-SCF₃ |
| I-1062 | Q.59 | 5-SO₂CF₃ |
| I-1063 | Q.59 | 5-CN |
| I-1064 | Q.59 | 5-OCF₃ |
| I-1065 | Q.60 | — |
| I-1066 | Q.60 | 3-F |
| I-1067 | Q.60 | 3-Cl |
| I-1068 | Q.60 | 3-Br |
| I-1069 | Q.60 | 3-CH₃ |
| I-1070 | Q.60 | 3-OCH₃ |
| I-1071 | Q.60 | 3-CF₃ |
| I-1072 | Q.61 | 3-F, 5-F |
| I-1073 | Q.61 | 3-F, 5-Cl |
| I-1074 | Q.61 | 3-F, 5-Br |
| I-1075 | Q.61 | 3-F, 5-CH₃ |
| I-1076 | Q.61 | 3-F, 5-OCH₃ |
| I-1077 | Q.61 | 3-F, 5-CF₃ |
| I-1078 | Q.61 | 3-F, 5-SO₂CH₃ |
| I-1079 | Q.61 | 3-F, 5-SCF₃ |
| I-1080 | Q.61 | 3-F, 5-SO₂CF₃ |
| I-1081 | Q.61 | 3-F, 5-CN |
| I-1082 | Q.61 | 3-F, 5-OCF₃ |
| I-1083 | Q.61 | 3-Cl, 5-F |
| I-1084 | Q.61 | 3-Cl, 5-Cl |
| I-1085 | Q.61 | 3-Cl, 5-Br |
| I-1086 | Q.61 | 3-Cl, 5-CH₃ |
| I-1087 | Q.61 | 3-Cl, 5-OCH₃ |
| I-1088 | Q.61 | 3-Cl, 5-CF₃ |
| I-1089 | Q.61 | 3-Cl, 5-SO₂CH₃ |
| I-1090 | Q.61 | 3-Cl, 5-SCF₃ |
| I-1091 | Q.61 | 3-Cl, 5-SO₂CF₃ |
| I-1092 | Q.61 | 3-Cl, 5-CN |
| I-1093 | Q.61 | 3-Cl, 5-OCF₃ |
| I-1094 | Q.61 | 3-Br, 5-F |
| I-1095 | Q.61 | 3-Br, 5-Cl |
| I-1096 | Q.61 | 3-Br, 5-Br |
| I-1097 | Q.61 | 3-Br, 5-CH₃ |
| I-1098 | Q.61 | 3-Br, 5-OCH₃ |
| I-1099 | Q.61 | 3-Br, 5-CF₃ |
| I-1100 | Q.61 | 3-Br, 5-SO₂CH₃ |
| I-1101 | Q.61 | 3-Br, 5-SCF₃ |
| I-1102 | Q.61 | 3-Br, 5-SO₂CF₃ |
| I-1103 | Q.61 | 3-Br, 5-CN |
| I-1104 | Q.61 | 3-Br, 5-OCF₃ |
| I-1105 | Q.61 | 3-CH₃, 5-F |
| I-1106 | Q.61 | 3-CH₃, 5-Cl |
| I-1107 | Q.61 | 3-CH₃, 5-Br |
| I-1108 | Q.61 | 3-CH₃, 5-CH₃ |
| I-1109 | Q.61 | 3-CH₃, 5-OCH₃ |
| I-1110 | Q.61 | 3-CH₃, 5-CF₃ |
| I-1111 | Q.61 | 3-CH₃, 5-SO₂CH₃ |
| I-1112 | Q.61 | 3-CH₃, 5-SCF₃ |
| I-1113 | Q.61 | 3-CH₃, 5-SO₂CF₃ |
| I-1114 | Q.61 | 3-CH₃, 5-CN |
| I-1115 | Q.61 | 3-CH₃, 5-OCF₃ |
| I-1116 | Q.61 | 3-OCH₃, 5-F |
| I-1117 | Q.61 | 3-OCH₃, 5-Cl |
| I-1118 | Q.61 | 3-OCH₃, 5-Br |
| I-1119 | Q.61 | 3-OCH₃, 5-CH₃ |
| I-1120 | Q.61 | 3-OCH₃, 5-OCH₃ |
| I-1121 | Q.61 | 3-OCH₃, 5-CF₃ |
| I-1122 | Q.61 | 3-OCH₃, 5-SO₂CH₃ |
| I-1123 | Q.61 | 3-OCH₃, 5-SCF₃ |
| I-1124 | Q.61 | 3-OCH₃, 5-SO₂CF₃ |
| I-1125 | Q.61 | 3-OCH₃, 5-CN |
| I-1126 | Q.61 | 3-OCH₃, 5-OCF₃ |
| I-1127 | Q.61 | 3-CF₃, 5-F |
| I-1128 | Q.61 | 3-CF₃, 5-Cl |
| I-1129 | Q.61 | 3-CF₃, 5-Br |
| I-1130 | Q.61 | 3-CF₃, 5-CH₃ |
| I-1131 | Q.61 | 3-CF₃, 5-OCH₃ |
| I-1132 | Q.61 | 3-CF₃, 5-CF₃ |
| I-1133 | Q.61 | 3-CF₃, 5-SO₂CH₃ |
| I-1134 | Q.61 | 3-CF₃, 5-SCF₃ |
| I-1135 | Q.61 | 3-CF₃, 5-SO₂CF₃ |
| I-1136 | Q.61 | 3-CF₃, 5-CN |
| I-1137 | Q.61 | 3-CF₃, 5-OCF₃ |
| I-1138 | Q.62 | 6-F, 5-F |
| I-1139 | Q.62 | 6-F, 5-Cl |
| I-1140 | Q.62 | 6-F, 5-Br |
| I-1141 | Q.62 | 6-F, 5-CH₃ |
| I-1142 | Q.62 | 6-F, 5-OCH₃ |
| I-1143 | Q.62 | 6-F, 5-CF₃ |
| I-1144 | Q.62 | 6-F, 5-SO₂CH₃ |
| I-1145 | Q.62 | 6-F, 5-SCF₃ |
| I-1146 | Q.62 | 6-F, 5-SO₂CF₃ |
| I-1147 | Q.62 | 6-F, 5-CN |
| I-1148 | Q.62 | 6-F, 5-OCF₃ |
| I-1149 | Q.62 | 6-Cl, 5-F |
| I-1150 | Q.62 | 6-Cl, 5-Cl |
| I-1151 | Q.62 | 6-Cl, 5-Br |
| I-1152 | Q.62 | 6-Cl, 5-CH₃ |
| I-1153 | Q.62 | 6-Cl, 5-OCH₃ |
| I-1154 | Q.62 | 6-Cl, 5-CF₃ |
| I-1155 | Q.62 | 6-Cl, 5-SO₂CH₃ |
| I-1156 | Q.62 | 6-Cl, 5-SCF₃ |
| I-1157 | Q.62 | 6-Cl, 5-SO₂CF₃ |
| I-1158 | Q.62 | 6-Cl, 5-CN |
| I-1159 | Q.62 | 6-Cl, 5-OCF₃ |
| I-1160 | Q.62 | 6-Br, 5-F |
| I-1161 | Q.62 | 6-Br, 5-Cl |
| I-1162 | Q.62 | 6-Br, 5-Br |
| I-1163 | Q.62 | 6-Br, 5-CH₃ |
| I-1164 | Q.62 | 6-Br, 5-OCH₃ |
| I-1165 | Q.62 | 6-Br, 5-CF₃ |
| I-1166 | Q.62 | 6-Br, 5-SO₂CH₃ |
| I-1167 | Q.62 | 6-Br, 5-SCF₃ |
| I-1168 | Q.62 | 6-Br, 5-SO₂CF₃ |
| I-1169 | Q.62 | 6-Br, 5-CN |
| I-1170 | Q.62 | 6-Br, 5-OCF₃ |
| I-1171 | Q.62 | 6-CH₃, 5-F |
| I-1172 | Q.62 | 6-CH₃, 5-Cl |
| I-1173 | Q.62 | 6-CH₃, 5-Br |
| I-1174 | Q.62 | 6-CH₃, 5-CH₃ |
| I-1175 | Q.62 | 6-CH₃, 5-OCH₃ |
| I-1176 | Q.62 | 6-CH₃, 5-CF₃ |
| I-1177 | Q.62 | 6-CH₃, 5-SO₂CH₃ |
| I-1178 | Q.62 | 6-CH₃, 5-SCF₃ |
| I-1179 | Q.62 | 6-CH₃, 5-SO₂CF₃ |
| I-1180 | Q.62 | 6-CH₃, 5-CN |
| I-1181 | Q.62 | 6-CH₃, 5-OCF₃ |
| I-1182 | Q.62 | 6-OCH₃, 5-F |
| I-1183 | Q.62 | 6-OCH₃, 5-Cl |
| I-1184 | Q.62 | 6-OCH₃, 5-Br |
| I-1185 | Q.62 | 6-OCH₃, 5-CH₃ |
| I-1186 | Q.62 | 6-OCH₃, 5-OCH₃ |
| I-1187 | Q.62 | 6-OCH₃, 5-CF₃ |
| I-1188 | Q.62 | 6-OCH₃, 5-SO₂CH₃ |
| I-1189 | Q.62 | 6-OCH₃, 5-SCF₃ |
| I-1190 | Q.62 | 6-OCH₃, 5-SO₂CF₃ |
| I-1191 | Q.62 | 6-OCH₃, 5-CN |
| I-1192 | Q.62 | 6-OCH₃, 5-OCF₃ |
| I-1193 | Q.62 | 6-CF₃, 5-F |
| I-1194 | Q.62 | 6-CF₃, 5-Cl |
| I-1195 | Q.62 | 6-CF₃, 5-Br |
| I-1196 | Q.62 | 6-CF₃, 5-CH₃ |
| I-1197 | Q.62 | 6-CF₃, 5-OCH₃ |
| I-1198 | Q.62 | 6-CF₃, 5-CF₃ |
| I-1199 | Q.62 | 6-CF₃, 5-SO₂CH₃ |
| I-1200 | Q.62 | 6-CF₃, 5-SCF₃ |
| I-1201 | Q.62 | 6-CF₃, 5-SO₂CF₃ |
| I-1202 | Q.62 | 6-CF₃, 5-CN |
| I-1203 | Q.62 | 6-CF₃, 5-OCF₃ |
| I-1204 | Q.63 | — |
| I-1205 | Q.64 | 4-F |
| I-1206 | Q.64 | 4-Cl |
| I-1207 | Q.64 | 4-Br |
| I-1208 | Q.64 | 4-CH₃ |
| I-1209 | Q.64 | 4-OCH₃ |
| I-1210 | Q.64 | 4-CF₃ |
| I-1211 | Q.65 | 5-F |
| I-1212 | Q.65 | 5-Cl |
| I-1213 | Q.65 | 5-Br |
| I-1214 | Q.65 | 5-CH₃ |
| I-1215 | Q.65 | 5-OCH₃ |
| I-1216 | Q.65 | 5-CF₃ |
| I-1217 | Q.65 | 5-SO₂CH₃ |
| I-1218 | Q.65 | 5-SCF₃ |
| I-1219 | Q.65 | 5-SO₂CF₃ |
| I-1220 | Q.65 | 5-CN |
| I-1221 | Q.65 | 5-OCF₃ |
| I-1222 | Q.66 | 4-F, 5-F |
| I-1223 | Q.66 | 4-F, 5-Cl |
| I-1224 | Q.66 | 4-F, 5-Br |
| I-1225 | Q.66 | 4-F, 5-CH₃ |
| I-1226 | Q.66 | 4-F, 5-OCH₃ |
| I-1227 | Q.66 | 4-F, 5-CF₃ |
| I-1228 | Q.66 | 4-F, 5-SO₂CH₃ |
| I-1229 | Q.66 | 4-F, 5-SCF₃ |
| I-1230 | Q.66 | 4-F, 5-SO₂CF₃ |
| I-1231 | Q.66 | 4-F, 5-CN |
| I-1232 | Q.66 | 4-F, 5-OCF₃ |
| I-1233 | Q.66 | 4-Cl, 5-F |
| I-1234 | Q.66 | 4-Cl, 5-Cl |
| I-1235 | Q.66 | 4-Cl, 5-Br |
| I-1236 | Q.66 | 4-Cl, 5-CH₃ |
| I-1237 | Q.66 | 4-Cl, 5-OCH₃ |
| I-1238 | Q.66 | 4-Cl, 5-CF₃ |
| I-1239 | Q.66 | 4-Cl, 5-SO₂CH₃ |
| I-1240 | Q.66 | 4-Cl, 5-SCF₃ |
| I-1241 | Q.66 | 4-Cl, 5-SO₂CF₃ |
| I-1242 | Q.66 | 4-Cl, 5-CN |
| I-1243 | Q.66 | 4-Cl, 5-OCF₃ |
| I-1244 | Q.66 | 4-Br, 5-F |
| I-1245 | Q.66 | 4-Br, 5-Cl |
| I-1246 | Q.66 | 4-Br, 5-Br |
| I-1247 | Q.66 | 4-Br, 5-CH₃ |
| I-1248 | Q.66 | 4-Br, 5-OCH₃ |
| I-1249 | Q.66 | 4-Br, 5-CF₃ |
| I-1250 | Q.66 | 4-Br, 5-SO₂CH₃ |
| I-1251 | Q.66 | 4-Br, 5-SCF₃ |
| I-1252 | Q.66 | 4-Br, 5-SO₂CF₃ |
| I-1253 | Q.66 | 4-Br, 5-CN |
| I-1254 | Q.66 | 4-Br, 5-OCF₃ |
| I-1255 | Q.66 | 4-CH₃, 5-F |
| I-1256 | Q.66 | 4-CH₃, 5-Cl |
| I-1257 | Q.66 | 4-CH₃, 5-Br |
| I-1258 | Q.66 | 4-CH₃, 5-CH₃ |
| I-1259 | Q.66 | 4-CH₃, 5-OCH₃ |
| I-1260 | Q.66 | 4-CH₃, 5-CF₃ |
| I-1261 | Q.66 | 4-CH₃, 5-SO₂CH₃ |
| I-1262 | Q.66 | 4-CH₃, 5-SCF₃ |
| I-1263 | Q.66 | 4-CH₃, 5-SO₂CF₃ |
| I-1264 | Q.66 | 4-CH₃, 5-CN |
| I-1265 | Q.66 | 4-CH₃, 5-OCF₃ |
| I-1266 | Q.66 | 4-OCH₃, 5-F |
| I-1267 | Q.66 | 4-OCH₃, 5-Cl |
| I-1268 | Q.66 | 4-OCH₃, 5-Br |
| I-1269 | Q.66 | 4-OCH₃, 5-CH₃ |
| I-1270 | Q.66 | 4-OCH₃, 5-OCH₃ |
| I-1271 | Q.66 | 4-OCH₃, 5-CF₃ |
| I-1272 | Q.66 | 4-OCH₃, 5-SO₂CH₃ |
| I-1273 | Q.66 | 4-OCH₃, 5-SCF₃ |
| I-1274 | Q.66 | 4-OCH₃, 5-SO₂CF₃ |
| I-1275 | Q.66 | 4-OCH₃, 5-CN |
| I-1276 | Q.66 | 4-OCH₃, 5-OCF₃ |
| I-1277 | Q.66 | 4-CF₃, 5-F |
| I-1278 | Q.66 | 4-CF₃, 5-Cl |
| I-1279 | Q.66 | 4-CF₃, 5-Br |
| I-1280 | Q.66 | 4-CF₃, 5-CH₃ |
| I-1281 | Q.66 | 4-CF₃, 5-OCH₃ |
| I-1282 | Q.66 | 4-CF₃, 5-CF₃ |
| I-1283 | Q.66 | 4-CF₃, 5-SO₂CH₃ |
| I-1284 | Q.66 | 4-CF₃, 5-SCF₃ |
| I-1285 | Q.66 | 4-CF₃, 5-SO₂CF₃ |
| I-1286 | Q.66 | 4-CF₃, 5-CN |
| I-1287 | Q.66 | 4-CF₃, 5-OCF₃ |
| I-1288 | Q.67 | — |
| I-1289 | Q.68 | 4-F |
| I-1290 | Q.68 | 4-Cl |
| I-1291 | Q.68 | 4-Br |
| I-1292 | Q.68 | 4-CH₃ |
| I-1293 | Q.68 | 4-OCH₃ |
| I-1294 | Q.68 | 4-CF₃ |
| I-1295 | Q.69 | 5-F |
| I-1296 | Q.69 | 5-Cl |
| I-1297 | Q.69 | 5-Br |
| I-1298 | Q.69 | 5-CH₃ |
| I-1299 | Q.69 | 5-OCH₃ |
| I-1300 | Q.69 | 5-CF₃ |
| I-1301 | Q.69 | 5-SO₂CH₃ |
| I-1302 | Q.69 | 5-SCF₃ |
| I-1303 | Q.69 | 5-SO₂CF₃ |
| I-1304 | Q.69 | 5-CN |
| I-1305 | Q.69 | 5-OCF₃ |
| I-1306 | Q.70 | 4-F, 5-F |
| I-1307 | Q.70 | 4-F, 5-Cl |
| I-1308 | Q.70 | 4-F, 5-Br |
| I-1309 | Q.70 | 4-F, 5-CH₃ |
| I-1310 | Q.70 | 4-F, 5-OCH₃ |
| I-1311 | Q.70 | 4-F, 5-CF₃ |
| I-1312 | Q.70 | 4-F, 5-SO₂CH₃ |
| I-1313 | Q.70 | 4-F, 5-SCF₃ |
| I-1314 | Q.70 | 4-F, 5-SO₂CF₃ |
| I-1315 | Q.70 | 4-F, 5-CN |
| I-1316 | Q.70 | 4-F, 5-OCF₃ |
| I-1317 | Q.70 | 4-Cl, 5-F |
| I-1318 | Q.70 | 4-Cl, 5-Cl |
| I-1319 | Q.70 | 4-Cl, 5-Br |
| I-1320 | Q.70 | 4-Cl, 5-CH₃ |
| I-1321 | Q.70 | 4-Cl, 5-OCH₃ |
| I-1322 | Q.70 | 4-Cl, 5-CF₃ |
| I-1323 | Q.70 | 4-Cl, 5-SO₂CH₃ |
| I-1324 | Q.70 | 4-Cl, 5-SCF₃ |
| I-1325 | Q.70 | 4-Cl, 5-SO₂CF₃ |
| I-1326 | Q.70 | 4-Cl, 5-CN |
| I-1327 | Q.70 | 4-Cl, 5-OCF₃ |
| I-1328 | Q.70 | 4-Br, 5-F |
| I-1329 | Q.70 | 4-Br, 5-Cl |
| I-1330 | Q.70 | 4-Br, 5-Br |
| I-1331 | Q.70 | 4-Br, 5-CH₃ |
| I-1332 | Q.70 | 4-Br, 5-OCH₃ |
| I-1333 | Q.70 | 4-Br, 5-CF₃ |
| I-1334 | Q.70 | 4-Br, 5-SO₂CH₃ |
| I-1335 | Q.70 | 4-Br, 5-SCF₃ |
| I-1336 | Q.70 | 4-Br, 5-SO₂CF₃ |
| I-1337 | Q.70 | 4-Br, 5-CN |
| I-1338 | Q.70 | 4-Br, 5-OCF₃ |
| I-1339 | Q.70 | 4-CH₃, 5-F |
| I-1340 | Q.70 | 4-CH₃, 5-Cl |
| I-1341 | Q.70 | 4-CH₃, 5-Br |
| I-1342 | Q.70 | 4-CH₃, 5-CH₃ |
| I-1343 | Q.70 | 4-CH₃, 5-OCH₃ |
| I-1344 | Q.70 | 4-CH₃, 5-CF₃ |
| I-1345 | Q.70 | 4-CH₃, 5-SO₂CH₃ |
| I-1346 | Q.70 | 4-CH₃, 5-SCF₃ |
| I-1347 | Q.70 | 4-CH₃, 5-SO₂CF₃ |
| I-1348 | Q.70 | 4-CH₃, 5-CN |
| I-1349 | Q.70 | 4-CH₃, 5-OCF₃ |
| I-1350 | Q.70 | 4-OCH₃, 5-F |
| I-1351 | Q.70 | 4-OCH₃, 5-Cl |
| I-1352 | Q.70 | 4-OCH₃, 5-Br |
| I-1353 | Q.70 | 4-OCH₃, 5-CH₃ |
| I-1354 | Q.70 | 4-OCH₃, 5-OCH₃ |
| I-1355 | Q.70 | 4-OCH₃, 5-CF₃ |
| I-1356 | Q.70 | 4-OCH₃, 5-SO₂CH₃ |
| I-1357 | Q.70 | 4-OCH₃, 5-SCF₃ |
| I-1358 | Q.70 | 4-OCH₃, 5-SO₂CF₃ |
| I-1359 | Q.70 | 4-OCH₃, 5-CN |
| I-1360 | Q.70 | 4-OCH₃, 5-OCF₃ |
| I-1361 | Q.70 | 4-CF₃, 5-F |
| I-1362 | Q.70 | 4-CF₃, 5-Cl |
| I-1363 | Q.70 | 4-CF₃, 5-Br |
| I-1364 | Q.70 | 4-CF₃, 5-CH₃ |
| I-1365 | Q.70 | 4-CF₃, 5-OCH₃ |
| I-1366 | Q.70 | 4-CF₃, 5-CF₃ |
| I-1367 | Q.70 | 4-CF₃, 5-SO₂CH₃ |
| I-1368 | Q.70 | 4-CF₃, 5-SCF₃ |
| I-1369 | Q.70 | 4-CF₃, 5-SO₂CF₃ |
| I-1370 | Q.70 | 4-CF₃, 5-CN |
| I-1371 | Q.70 | 4-CF₃, 5-OCF₃ |
| I-1372 | Q.71 | — |
| I-1373 | Q.72 | 5-F |
| I-1374 | Q.72 | 5-Cl |
| I-1375 | Q.72 | 5-Br |
| I-1376 | Q.72 | 5-CH₃ |
| I-1377 | Q.72 | 5-OCH₃ |
| I-1378 | Q.72 | 5-CF₃ |
| I-1379 | Q.72 | 5-SO₂CH₃ |
| I-1380 | Q.72 | 5-SCF₃ |
| I-1381 | Q.72 | 5-SO₂CF₃ |
| I-1382 | Q.72 | 5-CN |
| I-1383 | Q.73 | 5-OCF₃ |
| I-1384 | Q.73 | 4-F |
| I-1385 | Q.73 | 4-Cl |
| I-1386 | Q.73 | 4-Br |
| I-1387 | Q.73 | 4-CH₃ |
| I-1388 | Q.73 | 4-OCH₃ |
| I-1389 | Q.73 | 4-CF₃ |
| I-1390 | Q.74 | 3-F, 5-F |
| I-1391 | Q.74 | 3-F, 5-Cl |
| I-1392 | Q.74 | 3-F, 5-Br |
| I-1393 | Q.74 | 3-F, 5-CH₃ |
| I-1394 | Q.74 | 3-F, 5-OCH₃ |
| I-1395 | Q.74 | 3-F, 5-CF₃ |
| I-1396 | Q.74 | 3-F, 5-SO₂CH₃ |
| I-1397 | Q.74 | 3-F, 5-SCF₃ |
| I-1398 | Q.74 | 3-F, 5-SO₂CF₃ |
| I-1399 | Q.74 | 3-F, 5-CN |
| I-1400 | Q.74 | 3-F, 5-OCF₃ |
| I-1401 | Q.74 | 3-Cl, 5-F |
| I-1402 | Q.74 | 3-Cl, 5-Cl |
| I-1403 | Q.74 | 3-Cl, 5-Br |
| I-1404 | Q.74 | 3-Cl, 5-CH₃ |
| I-1405 | Q.74 | 3-Cl, 5-OCH₃ |
| I-1406 | Q.74 | 3-Cl, 5-CF₃ |
| I-1407 | Q.74 | 3-Cl, 5-SO₂CH₃ |
| I-1408 | Q.74 | 3-Cl, 5-SCF₃ |
| I-1409 | Q.74 | 3-Cl, 5-SO₂CF₃ |
| I-1410 | Q.74 | 3-Cl, 5-CN |
| I-1411 | Q.74 | 3-Cl, 5-OCF₃ |
| I-1412 | Q.74 | 3-Br, 5-F |
| I-1413 | Q.74 | 3-Br, 5-Cl |
| I-1414 | Q.74 | 3-Br, 5-Br |
| I-1415 | Q.74 | 3-Br, 5-CH₃ |
| I-1416 | Q.74 | 3-Br, 5-OCH₃ |
| I-1417 | Q.74 | 3-Br, 5-CF₃ |
| I-1418 | Q.74 | 3-Br, 5-SO₂CH₃ |
| I-1419 | Q.74 | 3-Br, 5-SCF₃ |
| I-1420 | Q.74 | 3-Br, 5-SO₂CF₃ |
| I-1421 | Q.74 | 3-Br, 5-CN |
| I-1422 | Q.74 | 3-Br, 5-OCF₃ |
| I-1423 | Q.74 | 3-CH₃, 5-F |
| I-1424 | Q.74 | 3-CH₃, 5-Cl |
| I-1425 | Q.74 | 3-CH₃, 5-Br |
| I-1426 | Q.74 | 3-CH₃, 5-CH₃ |
| I-1427 | Q.74 | 3-CH₃, 5-OCH₃ |
| I-1428 | Q.74 | 3-CH₃, 5-CF₃ |
| I-1429 | Q.74 | 3-CH₃, 5-SO₂CH₃ |
| I-1430 | Q.74 | 3-CH₃, 5-SCF₃ |
| I-1431 | Q.74 | 3-CH₃, 5-SO₂CF₃ |
| I-1432 | Q.74 | 3-CH₃, 5-CN |
| I-1433 | Q.74 | 3-CH₃, 5-OCF₃ |
| I-1434 | Q.74 | 3-OCH₃, 5-F |
| I-1435 | Q.74 | 3-OCH₃, 5-Cl |
| I-1436 | Q.74 | 3-OCH₃, 5-Br |
| I-1437 | Q.74 | 3-OCH₃, 5-CH₃ |
| I-1438 | Q.74 | 3-OCH₃, 5-OCH₃ |
| I-1439 | Q.74 | 3-OCH₃, 5-CF₃ |
| I-1440 | Q.74 | 3-OCH₃, 5-SO₂CH₃ |
| I-1441 | Q.74 | 3-OCH₃, 5-SCF₃ |
| I-1442 | Q.74 | 3-OCH₃, 5-SO₂CF₃ |
| I-1443 | Q.74 | 3-OCH₃, 5-CN |
| I-1444 | Q.74 | 3-OCH₃, 5-OCF₃ |
| I-1445 | Q.74 | 3-CF₃, 5-F |
| I-1446 | Q.74 | 3-CF₃, 5-Cl |
| I-1447 | Q.74 | 3-CF₃, 5-Br |
| I-1448 | Q.74 | 3-CF₃, 5-CH₃ |
| I-1449 | Q.74 | 3-CF₃, 5-OCH₃ |
| I-1450 | Q.74 | 3-CF₃, 5-CF₃ |
| I-1451 | Q.74 | 3-CF₃, 5-SO₂CH₃ |
| I-1452 | Q.74 | 3-CF₃, 5-SCF₃ |
| I-1453 | Q.74 | 3-CF₃, 5-SO₂CF₃ |
| I-1454 | Q.74 | 3-CF₃, 5-CN |
| I-1455 | Q.74 | 3-CF₃, 5-OCF₃ |
| I-1456 | Q.75 | — |
| I-1457 | Q.76 | 5-F |
| I-1458 | Q.76 | 5-Cl |
| I-1459 | Q.76 | 5-Br |
| I-1460 | Q.76 | 5-CH₃ |
| I-1461 | Q.76 | 5-OCH₃ |
| I-1462 | Q.76 | 5-CF₃ |
| I-1463 | Q.76 | 5-SO₂CH₃ |
| I-1464 | Q.76 | 5-SCF₃ |
| I-1465 | Q.76 | 5-SO₂CF₃ |
| I-1466 | Q.76 | 5-CN |
| I-1467 | Q.76 | 5-OCF₃ |
| I-1468 | Q.77 | 3-F |
| I-1469 | Q.77 | 3-Cl |
| I-1470 | Q.77 | 3-Br |
| I-1471 | Q.77 | 3-CH₃ |
| I-1472 | Q.77 | 3-OCH₃ |
| I-1473 | Q.77 | 3-CF₃ |
| I-1474 | Q.78 | 5-F, 3-F |
| I-1475 | Q.78 | 5-F, 3-Cl |
| I-1476 | Q.78 | 5-F, 3-Br |
| I-1477 | Q.78 | 5-F, 3-CH₃ |
| I-1478 | Q.78 | 5-F, 3-OCH₃ |
| I-1479 | Q.78 | 5-F, 3-CF₃ |
| I-1480 | Q.78 | 5-F, 3-SO₂CH₃ |
| I-1481 | Q.78 | 5-F, 3-SCF₃ |
| I-1482 | Q.78 | 5-F, 3-SO₂CF₃ |
| I-1483 | Q.78 | 5-F, 3-CN |
| I-1484 | Q.78 | 5-F, 3-OCF₃ |
| I-1485 | Q.78 | 5-Cl, 3-F |
| I-1486 | Q.78 | 5-Cl, 3-Cl |
| I-1487 | Q.78 | 5-Cl, 3-Br |
| I-1488 | Q.78 | 5-Cl, 3-CH₃ |
| I-1489 | Q.78 | 5-Cl, 3-OCH₃ |
| I-1490 | Q.78 | 5-Cl, 3-CF₃ |
| I-1491 | Q.78 | 5-Cl, 3-SO₂CH₃ |
| I-1492 | Q.78 | 5-Cl, 3-SCF₃ |
| I-1493 | Q.78 | 5-Cl, 3-SO₂CF₃ |
| I-1494 | Q.78 | 5-Cl, 3-CN |
| I-1495 | Q.78 | 5-Cl, 3-OCF₃ |
| I-1496 | Q.78 | 5-Br, 3-F |
| I-1497 | Q.78 | 5-Br, 3-Cl |
| I-1498 | Q.78 | 5-Br, 3-Br |
| I-1499 | Q.78 | 5-Br, 3-CH₃ |
| I-1500 | Q.78 | 5-Br, 3-OCH₃ |
| I-1501 | Q.78 | 5-Br, 3-CF₃ |
| I-1502 | Q.78 | 5-Br, 3-SO₂CH₃ |
| I-1503 | Q.78 | 5-Br, 3-SCF₃ |
| I-1504 | Q.78 | 5-Br, 3-SO₂CF₃ |
| I-1505 | Q.78 | 5-Br, 3-CN |
| I-1506 | Q.78 | 5-Br, 3-OCF₃ |
| I-1507 | Q.78 | 5-CH₃, 3-F |
| I-1508 | Q.78 | 5-CH₃, 3-Cl |
| I-1509 | Q.78 | 5-CH₃, 3-Br |
| I-1510 | Q.78 | 5-CH₃, 3-CH₃ |
| I-1511 | Q.78 | 5-CH₃, 3-OCH₃ |
| I-1512 | Q.78 | 5-CH₃, 3-CF₃ |
| I-1513 | Q.78 | 5-CH₃, 3-SO₂CH₃ |
| I-1514 | Q.78 | 5-CH₃, 3-SCF₃ |
| I-1515 | Q.78 | 5-CH₃, 3-SO₂CF₃ |
| I-1516 | Q.78 | 5-CH₃, 3-CN |
| I-1517 | Q.78 | 5-CH₃, 3-OCF₃ |
| I-1518 | Q.78 | 5-OCH₃, 3-F |
| I-1519 | Q.78 | 5-OCH₃, 3-Cl |
| I-1520 | Q.78 | 5-OCH₃, 3-Br |
| I-1521 | Q.78 | 5-OCH₃, 3-CH₃ |
| I-1522 | Q.78 | 5-OCH₃, 3-OCH₃ |
| I-1523 | Q.78 | 5-OCH₃, 3-CF₃ |
| I-1524 | Q.78 | 5-OCH₃, 3-SO₂CH₃ |
| I-1525 | Q.78 | 5-OCH₃, 3-SCF₃ |
| I-1526 | Q.78 | 5-OCH₃, 3-SO₂CF₃ |
| I-1527 | Q.78 | 5-OCH₃, 3-CN |
| I-1528 | Q.78 | 5-OCH₃, 3-OCF₃ |
| I-1529 | Q.78 | 5-CF₃, 3-F |
| I-1530 | Q.78 | 5-CF₃, 3-Cl |
| I-1531 | Q.78 | 5-CF₃, 3-Br |
| I-1532 | Q.78 | 5-CF₃, 3-CH₃ |
| I-1533 | Q.78 | 5-CF₃, 3-OCH₃ |
| I-1534 | Q.78 | 5-CF₃, 3-CF₃ |
| I-1535 | Q.78 | 5-CF₃, 3-SO₂CH₃ |
| I-1536 | Q.78 | 5-CF₃, 3-SCF₃ |
| I-1537 | Q.78 | 5-CF₃, 3-SO₂CF₃ |
| I-1538 | Q.78 | 5-CF₃, 3-CN |
| I-1539 | Q.78 | 5-CF₃, 3-OCF₃ |
| I-1540 | Q.163 | — |
| I-1541 | Q.167 | 2-F, 3-F |
| I-1542 | Q.167 | 2-F, 3-Cl |
| I-1543 | Q.167 | 2-F, 3-Br |
| I-1544 | Q.167 | 2-F, 3-CH₃ |
| I-1545 | Q.167 | 2-F, 3-OCH₃ |
| I-1546 | Q.167 | 2-F, 3-CF₃ |
| I-1547 | Q.167 | 2-F, 3-SO₂CH₃ |
| I-1548 | Q.167 | 2-F, 3-SCF₃ |
| I-1549 | Q.167 | 2-F, 3-SO₂CF₃ |
| I-1550 | Q.167 | 2-F, 3-CN |
| I-1551 | Q.167 | 2-F, 3-OCF₃ |
| I-1552 | Q.167 | 2-Cl, 3-F |
| I-1553 | Q.167 | 2-Cl, 3-Cl |
| I-1554 | Q.167 | 2-Cl, 3-Br |
| I-1555 | Q.167 | 2-Cl, 3-CH₃ |
| I-1556 | Q.167 | 2-Cl, 3-OCH₃ |
| I-1557 | Q.167 | 2-Cl, 3-CF₃ |
| I-1558 | Q.167 | 2-Cl, 3-SO₂CH₃ |
| I-1559 | Q.167 | 2-Cl, 3-SCF₃ |
| I-1560 | Q.167 | 2-Cl, 3-SO₂CF₃ |
| I-1561 | Q.167 | 2-Cl, 3-CN |
| I-1562 | Q.167 | 2-Cl, 3-OCF₃ |
| I-1563 | Q.167 | 2-Br, 3-F |
| I-1564 | Q.167 | 2-Br, 3-Cl |
| I-1565 | Q.167 | 2-Br, 3-Br |
| I-1566 | Q.167 | 2-Br, 3-CH₃ |
| I-1567 | Q.167 | 2-Br, 3-OCH₃ |
| I-1568 | Q.167 | 2-Br, 3-CF₃ |
| I-1569 | Q.167 | 2-Br, 3-SO₂CH₃ |
| I-1570 | Q.167 | 2-Br, 3-SCF₃ |
| I-1571 | Q.167 | 2-Br, 3-SO₂CF₃ |
| I-1572 | Q.167 | 2-Br, 3-CN |
| I-1573 | Q.167 | 2-Br, 3-OCF₃ |
| I-1574 | Q.167 | 2-CH₃, 3-F |
| I-1575 | Q.167 | 2-CH₃, 3-Cl |
| I-1576 | Q.167 | 2-CH₃, 3-Br |
| I-1577 | Q.167 | 2-CH₃, 3-CH₃ |
| I-1578 | Q.167 | 2-CH₃, 3-OCH₃ |
| I-1579 | Q.167 | 2-CH₃, 3-CF₃ |
| I-1580 | Q.167 | 2-CH₃, 3-SO₂CH₃ |
| I-1581 | Q.167 | 2-CH₃, 3-SCF₃ |
| I-1582 | Q.167 | 2-CH₃, 3-SO₂CF₃ |
| I-1583 | Q.167 | 2-CH₃, 3-CN |
| I-1584 | Q.167 | 2-CH₃, 3-OCF₃ |
| I-1585 | Q.167 | 2-OCH₃, 3-F |
| I-1586 | Q.167 | 2-OCH₃, 3-Cl |
| I-1587 | Q.167 | 2-OCH₃, 3-Br |
| I-1588 | Q.167 | 2-OCH₃, 3-CH₃ |
| I-1589 | Q.167 | 2-OCH₃, 3-OCH₃ |
| I-1590 | Q.167 | 2-OCH₃, 3-CF₃ |
| I-1591 | Q.167 | 2-OCH₃, 3-SO₂CH₃ |
| I-1592 | Q.167 | 2-OCH₃, 3-SCF₃ |
| I-1593 | Q.167 | 2-OCH₃, 3-SO₂CF₃ |
| I-1594 | Q.167 | 2-OCH₃, 3-CN |
| I-1595 | Q.167 | 2-OCH₃, 3-OCF₃ |
| I-1596 | Q.167 | 2-CF₃, 3-F |
| I-1597 | Q.167 | 2-CF₃, 3-Cl |
| I-1598 | Q.167 | 2-CF₃, 3-Br |
| I-1599 | Q.167 | 2-CF₃, 3-CH₃ |
| I-1600 | Q.167 | 2-CF₃, 3-OCH₃ |
| I-1601 | Q.167 | 2-CF₃, 3-CF₃ |
| I-1602 | Q.167 | 2-CF₃, 3-SO₂CH₃ |
| I-1603 | Q.167 | 2-CF₃, 3-SCF₃ |
| I-1604 | Q.167 | 2-CF₃, 3-SO₂CF₃ |
| I-1605 | Q.167 | 2-CF₃, 3-CN |
| I-1606 | Q.167 | 2-CF₃, 3-OCF₃ |
| I-1607 | Q.168 | 2-F, 4-F |
| I-1608 | Q.168 | 2-F, 4-Cl |
| I-1609 | Q.168 | 2-F, 4-Br |
| I-1610 | Q.168 | 2-F, 4-CH₃ |
| I-1611 | Q.168 | 2-F, 4-OCH3 |
| I-1612 | Q.168 | 2-F, 4-CF₃ |
| I-1613 | Q.168 | 2-F, 4-SO₂CH₃ |
| I-1614 | Q.168 | 2-F, 4-SCF₃ |
| I-1615 | Q.168 | 2-F, 4-SO₂CF₃ |
| I-1616 | Q.168 | 2-F, 4-CN |
| I-1617 | Q.168 | 2-F, 4-OCF₃ |
| I-1618 | Q.168 | 2-Cl, 4-F |
| I-1619 | Q.168 | 2-Cl, 4-Cl |
| I-1620 | Q.168 | 2-Cl, 4-Br |
| I-1621 | Q.168 | 2-Cl, 4-CH₃ |
| I-1622 | Q.168 | 2-Cl, 4-OCH₃ |
| I-1623 | Q.168 | 2-Cl, 4-CF₃ |
| I-1624 | Q.168 | 2-Cl, 4-SO₂CH₃ |
| I-1625 | Q.168 | 2-Cl, 4-SCF₃ |
| I-1626 | Q.168 | 2-Cl, 4-SO₂CF₃ |
| I-1627 | Q.168 | 2-Cl, 4-CN |
| I-1628 | Q.168 | 2-Cl, 4-OCF₃ |
| I-1629 | Q.168 | 2-Br, 4-F |
| I-1630 | Q.168 | 2-Br, 4-Cl |
| I-1631 | Q.168 | 2-Br, 4-Br |
| I-1632 | Q.168 | 2-Br, 4-CH₃ |
| I-1633 | Q.168 | 2-Br, 4-OCH₃ |
| I-1634 | Q.168 | 2-Br, 4-CF₃ |
| I-1635 | Q.168 | 2-Br, 4-SO₂CH₃ |
| I-1636 | Q.168 | 2-Br, 4-SCF₃ |
| I-1637 | Q.168 | 2-Br, 4-SO₂CF₃ |
| I-1638 | Q.168 | 2-Br, 4-CN |
| I-1639 | Q.168 | 2-Br, 4-OCF₃ |
| I-1640 | Q.168 | 2-CH₃, 4-F |
| I-1641 | Q.168 | 2-CH₃, 4-Cl |
| I-1642 | Q.168 | 2-CH₃, 4-Br |
| I-1643 | Q.168 | 2-CH₃, 4-CH₃ |
| I-1644 | Q.168 | 2-CH₃, 4-OCH₃ |
| I-1645 | Q.168 | 2-CH₃, 4-CF₃ |
| I-1646 | Q.168 | 2-CH₃, 4-SO₂CH₃ |
| I-1647 | Q.168 | 2-CH₃, 4-SCF₃ |
| I-1648 | Q.168 | 2-CH₃, 4-SO₂CF₃ |
| I-1649 | Q.168 | 2-CH₃, 4-CN |
| I-1650 | Q.168 | 2-CH₃, 4-OCF₃ |
| I-1651 | Q.168 | 2-OCH₃, 4-F |
| I-1652 | Q.168 | 2-OCH₃, 4-Cl |
| I-1653 | Q.168 | 2-OCH₃, 4-Br |
| I-1654 | Q.168 | 2-OCH₃, 4-CH₃ |
| I-1655 | Q.168 | 2-OCH₃, 4-OCH₃ |
| I-1656 | Q.168 | 2-OCH₃, 4-CF₃ |
| I-1657 | Q.168 | 2-OCH₃, 4-SO₂CH₃ |
| I-1658 | Q.168 | 2-OCH₃, 4-SCF₃ |
| I-1659 | Q.168 | 2-OCH₃, 4-SO₂CF₃ |
| I-1660 | Q.168 | 2-OCH₃, 4-CN |
| I-1661 | Q.168 | 2-OCH₃, 4-OCF₃ |
| I-1662 | Q.168 | 2-CF₃, 4-F |
| I-1663 | Q.168 | 2-CF₃, 4-Cl |
| I-1664 | Q.168 | 2-CF₃, 4-Br |
| I-1665 | Q.168 | 2-CF₃, 4-CH₃ |
| I-1666 | Q.168 | 2-CF₃, 4-OCH₃ |
| I-1667 | Q.168 | 2-CF₃, 4-CF₃ |
| I-1668 | Q.168 | 2-CF₃, 4-SO₂CH₃ |
| I-1669 | Q.168 | 2-CF₃, 4-SCF₃ |
| I-1670 | Q.168 | 2-CF₃, 4-SO₂CF₃ |
| I-1671 | Q.168 | 2-CF₃, 4-CN |
| I-1672 | Q.168 | 2-CF₃, 4-OCF₃ |
| I-1673 | Q.169 | 2-F, 5-F |
| I-1674 | Q.169 | 2-F, 5-Cl |
| I-1675 | Q.169 | 2-F, 5-Br |
| I-1676 | Q.169 | 2-F, 5-CH₃ |
| I-1677 | Q.169 | 2-F, 5-OCH₃ |
| I-1678 | Q.169 | 2-F, 5-CF₃ |
| I-1679 | Q.169 | 2-F, 5-SO₂CH₃ |
| I-1680 | Q.169 | 2-F, 5-SCF₃ |
| I-1681 | Q.169 | 2-F, 5-SO₂CF₃ |
| I-1682 | Q.169 | 2-F, 5-CN |
| I-1683 | Q.169 | 2-F, 5-OCF₃ |
| I-1684 | Q.169 | 2-Cl, 5-F |
| I-1685 | Q.169 | 2-Cl, 5-Cl |
| I-1686 | Q.169 | 2-Cl, 5-Br |
| I-1687 | Q.169 | 2-Cl, 5-CH₃ |
| I-1688 | Q.169 | 2-Cl, 5-OCH₃ |
| I-1689 | Q.169 | 2-Cl, 5-CF₃ |
| I-1690 | Q.169 | 2-Cl, 5-SO₂CH₃ |
| I-1691 | Q.169 | 2-Cl, 5-SCF₃ |
| I-1692 | Q.169 | 2-Cl, 5-SO₂CF₃ |
| I-1693 | Q.169 | 2-Cl, 5-CN |
| I-1694 | Q.169 | 2-Cl, 5-OCF₃ |
| I-1695 | Q.169 | 2-Br, 5-F |
| I-1696 | Q.169 | 2-Br, 5-Cl |
| I-1697 | Q.169 | 2-Br, 5-Br |
| I-1698 | Q.169 | 2-Br, 5-CH₃ |
| I-1699 | Q.169 | 2-Br, 5-OCH₃ |
| I-1700 | Q.169 | 2-Br, 5-CF₃ |
| I-1701 | Q.169 | 2-Br, 5-SO₂CH₃ |
| I-1702 | Q.169 | 2-Br, 5-SCF₃ |
| I-1703 | Q.169 | 2-Br, 5-SO₂CF₃ |
| I-1704 | Q.169 | 2-Br, 5-CN |
| I-1705 | Q.169 | 2-Br, 5-OCF₃ |
| I-1706 | Q.169 | 2-CH₃, 5-F |
| I-1707 | Q.169 | 2-CH₃, 5-Cl |
| I-1708 | Q.169 | 2-CH₃, 5-Br |
| I-1709 | Q.169 | 2-CH₃, 5-CH₃ |
| I-1710 | Q.169 | 2-CH₃, 5-OCH₃ |
| I-1711 | Q.169 | 2-CH₃, 5-CF₃ |
| I-1712 | Q.169 | 2-CH₃, 5-SO₂CH₃ |
| I-1713 | Q.169 | 2-CH₃, 5-SCF₃ |
| I-1714 | Q.169 | 2-CH₃, 5-SO₂CF₃ |
| I-1715 | Q.169 | 2-CH₃, 5-CN |
| I-1716 | Q.169 | 2-CH₃, 5-OCF₃ |
| I-1717 | Q.169 | 2-OCH₃, 5-F |
| I-1718 | Q.169 | 2-OCH₃, 5-Cl |
| I-1719 | Q.169 | 2-OCH₃, 5-Br |
| I-1720 | Q.169 | 2-OCH₃, 5-CH₃ |
| I-1721 | Q.169 | 2-OCH₃, 5-OCH₃ |
| I-1722 | Q.169 | 2-OCH₃, 5-CF₃ |
| I-1723 | Q.169 | 2-OCH₃, 5-SO₂CH₃ |
| I-1724 | Q.169 | 2-OCH₃, 5-SCF₃ |
| I-1725 | Q.169 | 2-OCH₃, 5-SO₂CF₃ |
| I-1726 | Q.169 | 2-OCH₃, 5-CN |
| I-1727 | Q.169 | 2-OCH₃, 5-OCF₃ |
| I-1728 | Q.169 | 2-CF₃, 5-F |
| I-1729 | Q.169 | 2-CF₃, 5-Cl |
| I-1730 | Q.169 | 2-CF₃, 5-Br |
| I-1731 | Q.169 | 2-CF₃, 5-CH₃ |
| I-1732 | Q.169 | 2-CF₃, 5-OCH₃ |
| I-1733 | Q.169 | 2-CF₃, 5-CF₃ |
| I-1734 | Q.169 | 2-CF₃, 5-SO₂CH₃ |
| I-1735 | Q.169 | 2-CF₃, 5-SCF₃ |
| I-1736 | Q.169 | 2-CF₃, 5-SO₂CF₃ |
| I-1737 | Q.169 | 2-CF₃, 5-CN |
| I-1738 | Q.169 | 2-CF₃, 5-OCF₃ |
| I-1739 | Q.170 | 2-F, 6-F |
| I-1740 | Q.170 | 2-F, 6-Cl |
| I-1741 | Q.170 | 2-F, 6-Br |
| I-1742 | Q.170 | 2-F, 6-CH₃ |
| I-1743 | Q.170 | 2-F, 6-OCH₃ |
| I-1744 | Q.170 | 2-F, 6-CF₃ |
| I-1745 | Q.170 | 2-F, 6-SO₂CH₃ |
| I-1746 | Q.170 | 2-F, 6-SCF₃ |
| I-1747 | Q.170 | 2-F, 6-SO₂CF₃ |
| I-1748 | Q.170 | 2-F, 6-CN |
| I-1749 | Q.170 | 2-F, 6-OCF₃ |
| I-1750 | Q.170 | 2-Cl, 6-F |
| I-1751 | Q.170 | 2-Cl, 6-Cl |
| I-1752 | Q.170 | 2-Cl, 6-Br |
| I-1753 | Q.170 | 2-Cl, 6-CH₃ |
| I-1754 | Q.170 | 2-Cl, 6-OCH₃ |
| I-1755 | Q.170 | 2-Cl, 6-CF₃ |
| I-1756 | Q.170 | 2-Cl, 6-SO₂CH₃ |
| I-1757 | Q.170 | 2-Cl, 6-SCF₃ |
| I-1758 | Q.170 | 2-Cl, 6-SO₂CF₃ |
| I-1759 | Q.170 | 2-Cl, 6-CN |
| I-1760 | Q.170 | 2-Cl, 6-OCF₃ |
| I-1761 | Q.170 | 2-Br, 6-F |
| I-1762 | Q.170 | 2-Br, 6-Cl |
| I-1763 | Q.170 | 2-Br, 6-Br |
| I-1764 | Q.170 | 2-Br, 6-CH₃ |
| I-1765 | Q.170 | 2-Br, 6-OCH₃ |
| I-1766 | Q.170 | 2-Br, 6-CF₃ |
| I-1767 | Q.170 | 2-Br, 6-SO₂CH₃ |
| I-1768 | Q.170 | 2-Br, 6-SCF₃ |
| I-1769 | Q.170 | 2-Br, 6-SO₂CF₃ |
| I-1770 | Q.170 | 2-Br, 6-CN |
| I-1771 | Q.170 | 2-Br, 6-OCF₃ |
| I-1772 | Q.170 | 2-CH₃, 6-F |
| I-1773 | Q.170 | 2-CH₃, 6-Cl |
| I-1774 | Q.170 | 2-CH₃, 6-Br |
| I-1775 | Q.170 | 2-CH₃, 6-CH₃ |
| I-1776 | Q.170 | 2-CH₃, 6-OCH₃ |
| I-1777 | Q.170 | 2-CH₃, 6-CF₃ |
| I-1778 | Q.170 | 2-CH₃, 6-SO₂CH₃ |
| I-1779 | Q.170 | 2-CH₃, 6-SCF₃ |
| I-1780 | Q.170 | 2-CH₃, 6-SO₂CF₃ |
| I-1781 | Q.170 | 2-CH₃, 6-CN |
| I-1782 | Q.170 | 2-CH₃, 6-OCF₃ |
| I-1783 | Q.170 | 2-OCH₃, 6-F |
| I-1784 | Q.170 | 2-OCH₃, 6-Cl |
| I-1785 | Q.170 | 2-OCH₃, 6-Br |
| I-1786 | Q.170 | 2-OCH₃, 6-CH₃ |
| I-1787 | Q.170 | 2-OCH₃, 6-OCH₃ |
| I-1788 | Q.170 | 2-OCH₃, 6-CF₃ |
| I-1789 | Q.170 | 2-OCH₃, 6-SO₂CH₃ |
| I-1790 | Q.170 | 2-OCH₃, 6-SCF₃ |
| I-1791 | Q.170 | 2-OCH₃, 6-SO₂CF₃ |
| I-1792 | Q.170 | 2-OCH₃, 6-CN |
| I-1793 | Q.170 | 2-OCH₃, 6-OCF₃ |
| I-1794 | Q.170 | 2-CF₃, 6-F |
| I-1795 | Q.170 | 2-CF₃, 6-Cl |
| I-1796 | Q.170 | 2-CF₃, 6-Br |
| I-1797 | Q.170 | 2-CF₃, 6-CH₃ |
| I-1798 | Q.170 | 2-CF₃, 6-OCH₃ |
| I-1799 | Q.170 | 2-CF₃, 6-CF₃ |
| I-1800 | Q.170 | 2-CF₃, 6-SO₂CH₃ |
| I-1801 | Q.170 | 2-CF₃, 6-SCF₃ |
| I-1802 | Q.170 | 2-CF₃, 6-SO₂CF₃ |
| I-1803 | Q.170 | 2-CF₃, 6-CN |
| I-1804 | Q.170 | 2-CF₃, 6-OCF₃ |
| I-1805 | Q.171 | 3-F, 4-F |
| I-1806 | Q.171 | 3-F, 4-Cl |
| I-1807 | Q.171 | 3-F, 4-Br |
| I-1808 | Q.171 | 3-F, 4-CH₃ |
| I-1809 | Q.171 | 3-F, 4-OCH₃ |
| I-1810 | Q.171 | 3-F, 4-CF₃ |
| I-1811 | Q.171 | 3-F, 4-SO₂CH₃ |
| I-1812 | Q.171 | 3-F, 4-SCF₃ |
| I-1813 | Q.171 | 3-F, 4-SO₂CF₃ |
| I-1814 | Q.171 | 3-F, 4-CN |
| I-1815 | Q.171 | 3-F, 4-OCF₃ |
| I-1816 | Q.171 | 3-Cl, 4-F |
| I-1817 | Q.171 | 3-Cl, 4-Cl |
| I-1818 | Q.171 | 3-Cl, 4-Br |
| I-1819 | Q.171 | 3-Cl, 4-CH₃ |
| I-1820 | Q.171 | 3-Cl, 4-OCH₃ |
| I-1821 | Q.171 | 3-Cl, 4-CF₃ |
| I-1822 | Q.171 | 3-Cl, 4-SO₂CH₃ |
| I-1823 | Q.171 | 3-Cl, 4-SCF₃ |
| I-1824 | Q.171 | 3-Cl, 4-SO₂CF₃ |
| I-1825 | Q.171 | 3-Cl, 4-CN |
| I-1826 | Q.171 | 3-Cl, 4-OCF₃ |
| I-1827 | Q.171 | 3-Br, 4-F |
| I-1828 | Q.171 | 3-Br, 4-Cl |
| I-1829 | Q.171 | 3-Br, 4-Br |
| I-1830 | Q.171 | 3-Br, 4-CH₃ |
| I-1831 | Q.171 | 3-Br, 4-OCH₃ |
| I-1832 | Q.171 | 3-Br, 4-CF₃ |
| I-1833 | Q.171 | 3-Br, 4-SO₂CH₃ |
| I-1834 | Q.171 | 3-Br, 4-SCF₃ |
| I-1835 | Q.171 | 3-Br, 4-SO₂CF₃ |
| I-1836 | Q.171 | 3-Br, 4-CN |
| I-1837 | Q.171 | 3-Br, 4-OCF₃ |
| I-1838 | Q.171 | 3-CH₃, 4-F |
| I-1839 | Q.171 | 3-CH₃, 4-Cl |
| I-1840 | Q.171 | 3-CH₃, 4-Br |
| I-1841 | Q.171 | 3-CH₃, 4-CH₃ |
| I-1842 | Q.171 | 3-CH₃, 4-OCH₃ |
| I-1843 | Q.171 | 3-CH₃, 4-CF₃ |
| I-1844 | Q.171 | 3-CH₃, 4-SO₂CH₃ |
| I-1845 | Q.171 | 3-CH₃, 4-SCF₃ |
| I-1846 | Q.171 | 3-CH₃, 4-SO₂CF₃ |
| I-1847 | Q.171 | 3-CH₃, 4-CN |
| I-1848 | Q.171 | 3-CH₃, 4-OCF₃ |
| I-1849 | Q.171 | 3-OCH₃, 4-F |
| I-1850 | Q.171 | 3-OCH₃, 4-Cl |
| I-1851 | Q.171 | 3-OCH₃, 4-Br |
| I-1852 | Q.171 | 3-OCH₃, 4-CH₃ |
| I-1853 | Q.171 | 3-OCH₃, 4-OCH₃ |
| I-1854 | Q.171 | 3-OCH₃, 4-CF₃ |
| I-1855 | Q.171 | 3-OCH₃, 4-SO₂CH₃ |
| I-1856 | Q.171 | 3-OCH₃, 4-SCF₃ |
| I-1857 | Q.171 | 3-OCH₃, 4-SO₂CF₃ |
| I-1858 | Q.171 | 3-OCH₃, 4-CN |
| I-1859 | Q.171 | 3-OCH₃, 4-OCF₃ |
| I-1860 | Q.171 | 3-CF₃, 4-F |
| I-1861 | Q.171 | 3-CF₃, 4-Cl |
| I-1862 | Q.171 | 3-CF₃, 4-Br |
| I-1863 | Q.171 | 3-CF₃, 4-CH₃ |
| I-1864 | Q.171 | 3-CF₃, 4-OCH₃ |
| I-1865 | Q.171 | 3-CF₃, 4-CF₃ |
| I-1866 | Q.171 | 3-CF₃, 4-SO₂CH₃ |
| I-1867 | Q.171 | 3-CF₃, 4-SCF₃ |
| I-1868 | Q.171 | 3-CF₃, 4-SO₂CF₃ |
| I-1869 | Q.171 | 3-CF₃, 4-CN |
| I-1870 | Q.171 | 3-CF₃, 4-OCF₃ |
| I-1871 | Q.171 | 3-CH₃, 4-F |
| I-1872 | Q.171 | 3-CH₃, 4-Cl |
| I-1873 | Q.171 | 3-CH₃, 4-Br |
| I-1874 | Q.171 | 3-CH₃, 4-CH₃ |
| I-1875 | Q.171 | 3-CH₃, 4-OCH₃ |
| I-1876 | Q.171 | 3-CH₃, 4-CF₃ |
| I-1877 | Q.171 | 3-CH₃, 4-SO₂CH₃ |
| I-1878 | Q.171 | 3-CH₃, 4-SCF₃ |
| I-1879 | Q.171 | 3-CH₃, 4-SO₂CF₃ |
| I-1880 | Q.171 | 3-CH₃, 4-CN |
| I-1881 | Q.171 | 3-CH₃, 4-OCF₃ |
| I-1882 | Q.171 | 3-SO₂CH₃, 4-F |
| I-1883 | Q.171 | 3-SO₂CH₃, 4-Cl |
| I-1884 | Q.171 | 3-SO₂CH₃, 4-Br |
| I-1885 | Q.171 | 3-SO₂CH₃, 4-CH₃ |
| I-1886 | Q.171 | 3-SO₂CH₃, 4-OCH₃ |
| I-1887 | Q.171 | 3-SO₂CH₃, 4-CF₃ |
| I-1888 | Q.171 | 3-SO₂CH₃, 4-SO₂CH₃ |
| I-1889 | Q.171 | 3-SO₂CH₃, 4-SCF₃ |
| I-1890 | Q.171 | 3-SO₂CH₃, 4-SO₂CF₃ |
| I-1891 | Q.171 | 3-SO₂CH₃, 4-CN |
| I-1892 | Q.171 | 3-SO₂CH₃, 4-OCF₃ |
| I-1893 | Q.171 | 3-SO₂CF₃, 4-F |
| I-1894 | Q.171 | 3-SO₂CF₃, 4-Cl |
| I-1895 | Q.171 | 3-SO₂CF₃, 4-Br |
| I-1896 | Q.171 | 3-SO₂CF₃, 4-CH₃ |
| I-1897 | Q.171 | 3-SO₂CF₃, 4-OCH₃ |
| I-1898 | Q.171 | 3-SO₂CF₃, 4-CF₃ |
| I-1899 | Q.171 | 3-SO₂CH₃, 4-SO₂CH₃ |
| I-1900 | Q.171 | 3-SO₂CF₃, 4-SCF₃ |
| I-1901 | Q.171 | 3-SO₂CF₃, 4-SO₂CF₃ |
| I-1902 | Q.171 | 3-SO₂CF₃, 4-CN |
| I-1903 | Q.171 | 3-SO₂CF₃, 4-OCF₃ |
| I-1904 | Q.171 | 3-SO₂CF₃, 4-F |
| I-1905 | Q.171 | 3-SO₂CF₃, 4-Cl |
| I-1906 | Q.171 | 3-SO₂CF₃, 4-Br |
| I-1907 | Q.171 | 3-SO₂CF₃, 4-CH₃ |
| I-1908 | Q.171 | 3-SO₂CF₃, 4-OCH₃ |
| I-1909 | Q.171 | 3-SO₂CF₃, 4-CF₃ |
| I-1910 | Q.171 | 3-SO₂CH₃, 4-SO₂CH₃ |
| I-1911 | Q.171 | 3-SO₂CF₃, 4-SCF₃ |
| I-1912 | Q.171 | 3-SO₂CF₃, 4-SO₂CF₃ |
| I-1913 | Q.171 | 3-SO₂CF₃, 4-CN |
| I-1914 | Q.171 | 3-SO₂CF₃, 4-OCF₃ |
| I-1915 | Q.171 | 3-SCF₃, 4-F |
| I-1916 | Q.171 | 3-SCF₃, 4-Cl |
| I-1917 | Q.171 | 3-SCF₃, 4-Br |
| I-1918 | Q.171 | 3-SCF₃, 4-CH₃ |
| I-1919 | Q.171 | 3-SCF₃, 4-OCH₃ |
| I-1920 | Q.171 | 3-SCF₃, 4-CF₃ |
| I-1921 | Q.171 | 3-SCH₃, 4-SO₂CH₃ |
| I-1922 | Q.171 | 3-SCF₃, 4-SCF₃ |
| I-1923 | Q.171 | 3-SCF₃, 4-SO₂CF₃ |
| I-1924 | Q.171 | 3-SCF₃, 4-CN |
| I-1925 | Q.171 | 3-SCF₃, 4-OCF₃ |
| I-1926 | Q.171 | 3-OCF₃, 4-F |
| I-1927 | Q.171 | 3-OCF₃, 4-Cl |
| I-1928 | Q.171 | 3-OCF₃, 4-Br |
| I-1929 | Q.171 | 3-OCF₃, 4-CH₃ |
| I-1930 | Q.171 | 3-OCF₃, 4-OCH₃ |
| I-1931 | Q.171 | 3-OCF₃, 4-CF₃ |
| I-1932 | Q.171 | 3-OCH₃, 4-SO₂CH₃ |
| I-1933 | Q.171 | 3-OCF₃, 4-SCF₃ |
| I-1934 | Q.171 | 3-OCF₃, 4-SO₂CF₃ |
| I-1935 | Q.171 | 3-OCF₃, 4-CN |
| I-1936 | Q.171 | 3-OCF₃, 4-OCF₃ |
| I-1937 | Q.171 | 3-CN, 4-F |
| I-1938 | Q.171 | 3-CN, 4-Cl |
| I-1939 | Q.171 | 3-CN, 4-Br |
| I-1940 | Q.171 | 3-CN, 4-CH₃ |
| I-1941 | Q.171 | 3-CN, 4-OCH₃ |
| I-1942 | Q.171 | 3-CN, 4-CF₃ |
| I-1943 | Q.171 | 3-CN, 4-SO₂CH₃ |
| I-1944 | Q.171 | 3-CN, 4-SCF₃ |
| I-1945 | Q.171 | 3-CN, 4-SO₂CF₃ |
| I-1946 | Q.171 | 3-CN, 4-CN |
| I-1947 | Q.171 | 3-CN, 4-OCF₃ |
| I-1948 | Q.172 | 3-F, 5-F |
| I-1949 | Q.172 | 3-F, 5-Cl |
| I-1950 | Q.172 | 3-F, 5-Br |
| I-1951 | Q.172 | 3-F, 5-CH₃ |
| I-1952 | Q.172 | 3-F, 5-OCH₃ |
| I-1953 | Q.172 | 3-F, 5-CF₃ |
| I-1954 | Q.172 | 3-F, 5-SO₂CH₃ |
| I-1955 | Q.172 | 3-F, 5-SCF₃ |
| I-1956 | Q.172 | 3-F, 5-SO₂CF₃ |
| I-1957 | Q.172 | 3-F, 5-CN |
| I-1958 | Q.172 | 3-F, 5-OCF₃ |
| I-1959 | Q.172 | 3-Cl, 5-F |
| I-1960 | Q.172 | 3-Cl, 5-Cl |
| I-1961 | Q.172 | 3-Cl, 5-Br |
| I-1962 | Q.172 | 3-Cl, 5-CH₃ |
| I-1963 | Q.172 | 3-Cl, 5-OCH₃ |
| I-1964 | Q.172 | 3-Cl, 5-CF₃ |
| I-1965 | Q.172 | 3-Cl, 5-SO₂CH₃ |
| I-1966 | Q.172 | 3-Cl, 5-SCF₃ |
| I-1967 | Q.172 | 3-Cl, 5-SO₂CF₃ |
| I-1968 | Q.172 | 3-Cl, 5-CN |
| I-1969 | Q.172 | 3-Cl, 5-OCF₃ |
| I-1970 | Q.172 | 3-Br, 5-F |
| I-1971 | Q.172 | 3-Br, 5-Cl |
| I-1972 | Q.172 | 3-Br, 5-Br |
| I-1973 | Q.172 | 3-Br, 5-CH₃ |
| I-1974 | Q.172 | 3-Br, 5-OCH₃ |
| I-1975 | Q.172 | 3-Br, 5-CF₃ |
| I-1976 | Q.172 | 3-Br, 5-SO₂CH₃ |
| I-1977 | Q.172 | 3-Br, 5-SCF₃ |
| I-1978 | Q.172 | 3-Br, 5-SO₂CF₃ |
| I-1979 | Q.172 | 3-Br, 5-CN |
| I-1980 | Q.172 | 3-Br, 5-OCF₃ |
| I-1981 | Q.172 | 3-CH₃, 5-F |
| I-1982 | Q.172 | 3-CH₃, 5-Cl |
| I-1983 | Q.172 | 3-CH₃, 5-Br |
| I-1984 | Q.172 | 3-CH₃, 5-CH₃ |
| I-1985 | Q.172 | 3-CH₃, 5-OCH₃ |
| I-1986 | Q.172 | 3-CH₃, 5-CF₃ |
| I-1987 | Q.172 | 3-CH₃, 5-SO₂CH₃ |
| I-1988 | Q.172 | 3-CH₃, 5-SCF₃ |
| I-1989 | Q.172 | 3-CH₃, 5-SO₂CF₃ |
| I-1990 | Q.172 | 3-CH₃, 5-CN |
| I-1991 | Q.172 | 3-CH₃, 5-OCF₃ |
| I-1992 | Q.172 | 3-OCH₃, 5-F |
| I-1993 | Q.172 | 3-OCH₃, 5-Cl |
| I-1994 | Q.172 | 3-OCH₃, 5-Br |
| I-1995 | Q.172 | 3-OCH₃, 5-CH₃ |
| I-1996 | Q.172 | 3-OCH₃, 5-OCH₃ |
| I-1997 | Q.172 | 3-OCH₃, 5-CF₃ |
| I-1998 | Q.172 | 3-OCH₃, 5-SO₂CH₃ |
| I-1999 | Q.172 | 3-OCH₃, 5-SCF₃ |
| I-2000 | Q.172 | 3-OCH₃, 5-SO₂CF₃ |
| I-2001 | Q.172 | 3-OCH₃, 5-CN |
| I-2002 | Q.172 | 3-OCH₃, 5-OCF₃ |
| I-2003 | Q.172 | 3-CF₃, 5-F |
| I-2004 | Q.172 | 3-CF₃, 5-Cl |
| I-2005 | Q.172 | 3-CF₃, 5-Br |
| I-2006 | Q.172 | 3-CF₃, 5-CH₃ |
| I-2007 | Q.172 | 3-CF₃, 5-OCH₃ |
| I-2008 | Q.172 | 3-CF₃, 5-CF₃ |
| I-2009 | Q.172 | 3-CF₃, 5-SO₂CH₃ |
| I-2010 | Q.172 | 3-CF₃, 5-SCF₃ |
| I-2011 | Q.172 | 3-CF₃, 5-SO₂CF₃ |
| I-2012 | Q.172 | 3-CF₃, 5-CN |
| I-2013 | Q.172 | 3-CF₃, 5-OCF₃ |
| I-2014 | Q.173 | 3-F, 5-F |
| I-2015 | Q.173 | 3-F, 5-Cl |
| I-2016 | Q.173 | 3-F, 5-Br |
| I-2017 | Q.173 | 3-F, 5-CH₃ |
| I-2018 | Q.173 | 3-F, 5-OCH₃ |
| I-2019 | Q.173 | 3-F, 5-CF₃ |
| I-2020 | Q.173 | 3-F, 5-SO₂CH₃ |
| I-2021 | Q.173 | 3-F, 5-SCF₃ |
| I-2022 | Q.173 | 3-F, 5-SO₂CF₃ |
| I-2023 | Q.173 | 3-F, 5-CN |
| I-2024 | Q.173 | 3-F, 5-OCF₃ |
| I-2025 | Q.173 | 3-Cl, 5-F |
| I-2026 | Q.173 | 3-Cl, 5-Cl |
| I-2027 | Q.173 | 3-Cl, 5-Br |
| I-2028 | Q.173 | 3-Cl, 5-CH₃ |
| I-2029 | Q.173 | 3-Cl, 5-OCH₃ |
| I-2030 | Q.173 | 3-Cl, 5-CF₃ |
| I-2031 | Q.173 | 3-Cl, 5-SO₂CH₃ |
| I-2032 | Q.173 | 3-Cl, 5-SCF₃ |
| I-2033 | Q.173 | 3-Cl, 5-SO₂CF₃ |
| I-2034 | Q.173 | 3-Cl, 5-CN |
| I-2035 | Q.173 | 3-Cl, 5-OCF₃ |
| I-2036 | Q.173 | 3-Br, 5-F |
| I-2037 | Q.173 | 3-Br, 5-Cl |
| I-2038 | Q.173 | 3-Br, 5-Br |
| I-2039 | Q.173 | 3-Br, 5-CH₃ |
| I-2040 | Q.173 | 3-Br, 5-OCH₃ |
| I-2041 | Q.173 | 3-Br, 5-CF₃ |
| I-2042 | Q.173 | 3-Br, 5-SO₂CH₃ |
| I-2043 | Q.173 | 3-Br, 5-SCF₃ |
| I-2044 | Q.173 | 3-Br, 5-SO₂CF₃ |
| I-2045 | Q.173 | 3-Br, 5-CN |
| I-2046 | Q.173 | 3-Br, 5-OCF₃ |
| I-2047 | Q.173 | 3-CH₃, 5-F |
| I-2048 | Q.173 | 3-CH₃, 5-Cl |
| I-2049 | Q.173 | 3-CH₃, 5-Br |
| I-2050 | Q.173 | 3-CH₃, 5-CH₃ |
| I-2051 | Q.173 | 3-CH₃, 5-OCH₃ |
| I-2052 | Q.173 | 3-CH₃, 5-CF₃ |
| I-2053 | Q.173 | 3-CH₃, 5-SO₂CH₃ |
| I-2054 | Q.173 | 3-CH₃, 5-SCF₃ |
| I-2055 | Q.173 | 3-CH₃, 5-SO₂CF₃ |
| I-2056 | Q.173 | 3-CH₃, 5-CN |
| I-2057 | Q.173 | 3-CH₃, 5-OCF₃ |
| I-2058 | Q.173 | 3-OCH₃, 5-F |
| I-2059 | Q.173 | 3-OCH₃, 5-Cl |
| I-2060 | Q.173 | 3-OCH₃, 5-Br |
| I-2061 | Q.173 | 3-OCH₃, 5-CH₃ |
| I-2062 | Q.173 | 3-OCH₃, 5-OCH₃ |
| I-2063 | Q.173 | 3-OCH₃, 5-CF₃ |
| I-2064 | Q.173 | 3-OCH₃, 5-SO₂CH₃ |
| I-2065 | Q.173 | 3-OCH₃, 5-SCF₃ |
| I-2066 | Q.173 | 3-OCH₃, 5-SO₂CF₃ |
| I-2067 | Q.173 | 3-OCH₃, 5-CN |
| I-2068 | Q.173 | 3-OCH₃, 5-OCF₃ |
| I-2069 | Q.173 | 3-CF₃, 5-F |
| I-2070 | Q.173 | 3-CF₃, 5-Cl |
| I-2071 | Q.173 | 3-CF₃, 5-Br |
| I-2072 | Q.173 | 3-CF₃, 5-CH₃ |
| I-2073 | Q.173 | 3-CF₃, 5-OCH₃ |
| I-2074 | Q.173 | 3-CF₃, 5-CF₃ |
| I-2075 | Q.173 | 3-CF₃, 5-SO₂CH₃ |
| I-2076 | Q.173 | 3-CF₃, 5-SCF₃ |
| I-2077 | Q.173 | 3-CF₃, 5-SO₂CF₃ |
| I-2078 | Q.173 | 3-CF₃, 5-CN |
| I-2079 | Q.173 | 3-CF₃, 5-OCF₃ |
| I-2080 | Q.173 | 3-CH₃, 5-F |
| I-2081 | Q.173 | 3-CH₃, 5-Cl |
| I-2082 | Q.173 | 3-CH₃, 5-Br |
| I-2083 | Q.173 | 3-CH₃, 5-CH₃ |
| I-2084 | Q.173 | 3-CH₃, 5-OCH₃ |
| I-2085 | Q.173 | 3-CH₃, 5-CF₃ |
| I-2086 | Q.173 | 3-CH₃, 5-SO₂CH₃ |
| I-2087 | Q.173 | 3-CH₃, 5-SCF₃ |
| I-2088 | Q.173 | 3-CH₃, 5-SO₂CF₃ |
| I-2089 | Q.173 | 3-CH₃, 5-CN |
| I-2090 | Q.173 | 3-CH₃, 5-OCF₃ |
| I-2091 | Q.173 | 3-SO₂CH₃, 5-F |
| I-2092 | Q.173 | 3-SO₂CH₃, 5-Cl |
| I-2093 | Q.173 | 3-SO₂CH₃, 5-Br |
| I-2094 | Q.173 | 3-SO₂CH₃, 5-CH₃ |
| I-2095 | Q.173 | 3-SO₂CH₃, 5-OCH₃ |
| I-2096 | Q.173 | 3-SO₂CH₃, 5-CF₃ |
| I-2097 | Q.173 | 3-SO₂CH₃, 5-SO₂CH₃ |
| I-2098 | Q.173 | 3-SO₂CH₃, 5-SCF₃ |
| I-2099 | Q.173 | 3-SO₂CH₃, 5-SO₂CF₃ |
| I-2100 | Q.173 | 3-SO₂CH₃, 5-CN |
| I-2101 | Q.173 | 3-SO₂CH₃, 5-OCF₃ |
| I-2102 | Q.173 | 3-SO₂CF₃, 5-F |
| I-2103 | Q.173 | 3-SO₂CF₃, 5-Cl |
| I-2104 | Q.173 | 3-SO₂CF₃, 5-Br |
| I-2105 | Q.173 | 3-SO₂CF₃, 5-CH₃ |
| I-2106 | Q.173 | 3-SO₂CF₃, 5-OCH₃ |
| I-2107 | Q.173 | 3-SO₂CF₃, 5-CF₃ |
| I-2108 | Q.173 | 3-SO₂CH₃, 5-SO₂CH₃ |
| I-2109 | Q.173 | 3-SO₂CF₃, 5-SCF₃ |
| I-2110 | Q.173 | 3-SO₂CF₃, 5-SO₂CF₃ |
| I-2111 | Q.173 | 3-SO₂CF₃, 5-CN |
| I-2112 | Q.173 | 3-SO₂CF₃, 5-OCF₃ |
| I-2113 | Q.173 | 3-SO₂CF₃, 5-F |
| I-2114 | Q.173 | 3-SO₂CF₃, 5-Cl |
| I-2115 | Q.173 | 3-SO₂CF₃, 5-Br |
| I-2116 | Q.173 | 3-SO₂CF₃, 5-CH₃ |
| I-2117 | Q.173 | 3-SO₂CF₃, 5-OCH₃ |
| I-2118 | Q.173 | 3-SO₂CF₃, 5-CF₃ |
| I-2119 | Q.173 | 3-SO₂CH₃, 5-SO₂CH₃ |
| I-2120 | Q.173 | 3-SO₂CF₃, 5-SCF₃ |
| I-2121 | Q.173 | 3-SO₂CF₃, 5-SO₂CF₃ |
| I-2122 | Q.173 | 3-SO₂CF₃, 5-CN |
| I-2123 | Q.173 | 3-SO₂CF₃, 5-OCF₃ |
| I-2124 | Q.173 | 3-SCF₃, 5-F |
| I-2125 | Q.173 | 3-SCF₃, 5-Cl |
| I-2126 | Q.173 | 3-SCF₃, 5-Br |
| I-2127 | Q.173 | 3-SCF₃, 5-CH₃ |
| I-2128 | Q.173 | 3-SCF₃, 5-OCH₃ |
| I-2129 | Q.173 | 3-SCF₃, 5-CF₃ |
| I-2130 | Q.173 | 3-SCH₃, 5-SO₂CH₃ |
| I-2131 | Q.173 | 3-SCF₃, 5-SCF₃ |
| I-2132 | Q.173 | 3-SCF₃, 5-SO₂CF₃ |
| I-2133 | Q.173 | 3-SCF₃, 5-CN |
| I-2134 | Q.173 | 3-SCF₃, 5-OCF₃ |
| I-2135 | Q.173 | 3-OCF₃, 5-F |
| I-2136 | Q.173 | 3-OCF₃, 5-Cl |
| I-2137 | Q.173 | 3-OCF₃, 5-Br |
| I-2138 | Q.173 | 3-OCF₃, 5-CH₃ |
| I-2139 | Q.173 | 3-OCF₃, 5-OCH₃ |
| I-2140 | Q.173 | 3-OCF₃, 5-CF₃ |
| I-2141 | Q.173 | 3-OCH₃, 5-SO₂CH₃ |
| I-2142 | Q.173 | 3-OCF₃, 5-SCF₃ |
| I-2143 | Q.173 | 3-OCF₃, 5-SO₂CF₃ |
| I-2144 | Q.173 | 3-OCF₃, 5-CN |
| I-2145 | Q.173 | 3-OCF₃, 5-OCF₃ |
| I-2146 | Q.173 | 3-CN, 5-F |
| I-2147 | Q.173 | 3-CN, 5-Cl |
| I-2148 | Q.173 | 3-CN, 5-Br |
| I-2149 | Q.173 | 3-CN, 5-CH₃ |
| I-2150 | Q.173 | 3-CN, 5-OCH₃ |
| I-2151 | Q.173 | 3-CN, 5-CF₃ |
| I-2152 | Q.173 | 3-CN, 5-SO₂CH₃ |
| I-2153 | Q.173 | 3-CN, 5-SCF₃ |
| I-2154 | Q.173 | 3-CN, 5-SO₂CF₃ |
| I-2155 | Q.173 | 3-CN, 5-CN |
| I-2156 | Q.173 | 3-CN, 5-OCF₃ |
| I-2157 | Q.193 | — |
| I-2158 | Q.194 | 2-F |
| I-2159 | Q.194 | 2-Cl |
| I-2160 | Q.194 | 2-Br |
| I-2161 | Q.194 | 2-CH₃ |
| I-2162 | Q.194 | 2-OCH₃ |
| I-2163 | Q.194 | 2-CF₃ |
| I-2164 | Q.194 | 2-SCF₃ |
| I-2165 | Q.194 | 2-CN |
| I-2166 | Q.194 | 2-OCF₃ |
| I-2167 | Q.195 | — |
| I-2168 | Q.196 | 2-F |
| I-2169 | Q.196 | 2-Cl |
| I-2170 | Q.196 | 2-Br |
| I-2171 | Q.196 | 2-CH₃ |
| I-2172 | Q.196 | 2-OCH₃ |
| I-2173 | Q.196 | 2-CF₃ |
| I-2174 | Q.196 | 2-SCF₃ |
| I-2175 | Q.196 | 2-CN |
| I-2176 | Q.196 | 2-OCF₃ |
| I-2177 | Q.197 | — |
| I-2178 | Q.198 | 2-F |
| I-2179 | Q.198 | 2-Cl |
| I-2180 | Q.198 | 2-Br |
| I-2181 | Q.198 | 2-CH₃ |
| I-2182 | Q.198 | 2-OCH₃ |
| I-2183 | Q.198 | 2-CF₃ |
| I-2184 | Q.198 | 2-SCF₃ |
| I-2185 | Q.198 | 2-CN |
| I-2186 | Q.198 | 2-OCF₃ |
| I-2187 | Q.199 | — |
| I-2188 | Q.200 | 2-F |
| I-2189 | Q.200 | 2-Cl |
| I-2190 | Q.200 | 2-Br |
| I-2191 | Q.200 | 2-CH₃ |
| I-2192 | Q.200 | 2-OCH₃ |
| I-2193 | Q.200 | 2-CF₃ |
| I-2194 | Q.200 | 2-SCF₃ |
| I-2195 | Q.200 | 2-CN |
| I-2196 | Q.200 | 2-OCF₃ |
| I-2197 | Q.201 | — |
| I-2198 | Q.202 | 2-F |
| I-2199 | Q.202 | 2-Cl |
| I-2200 | Q.202 | 2-Br |
| I-2201 | Q.202 | 2-CH₃ |
| I-2202 | Q.202 | 2-OCH₃ |
| I-2203 | Q.202 | 2-CF₃ |
| I-2204 | Q.202 | 2-SCF₃ |
| I-2205 | Q.202 | 2-CN |
| I-2206 | Q.202 | 2-OCF₃ |
| I-2207 | Q.203 | — |
| I-2208 | Q.204 | 2-F |
| I-2209 | Q.204 | 2-Cl |
| I-2210 | Q.204 | 2-Br |
| I-2211 | Q.204 | 2-CH₃ |
| I-2212 | Q.204 | 2-OCH₃ |
| I-2213 | Q.204 | 2-CF₃ |
| I-2214 | Q.204 | 2-SCF₃ |
| I-2215 | Q.204 | 2-CN |
| I-2216 | Q.204 | 2-OCF₃ |
| I-2217 | Q.205 | — |
| I-2218 | Q.206 | 2-F |
| I-2219 | Q.206 | 2-Cl |
| I-2220 | Q.206 | 2-Br |
| I-2221 | Q.206 | 2-CH₃ |
| I-2222 | Q.206 | 2-OCH₃ |
| I-2223 | Q.206 | 2-CF₃ |
| I-2224 | Q.206 | 2-SCF₃ |
| I-2225 | Q.206 | 2-CN |
| I-2226 | Q.206 | 2-OCF₃ |
| I-2227 | Q.207 | — |
| I-2228 | Q.208 | 2-F |
| I-2229 | Q.208 | 2-Cl |
| I-2230 | Q.208 | 2-Br |
| I-2231 | Q.208 | 2-CH₃ |
| I-2232 | Q.208 | 2-OCH₃ |
| I-2233 | Q.208 | 2-CF₃ |
| I-2234 | Q.208 | 2-SCF₃ |
| I-2235 | Q.208 | 2-CN |
| I-2236 | Q.208 | 2-OCF₃ |

| No. | R¹ | R_{f} | R³ |
|---|---|---|---|
| I-2237 | 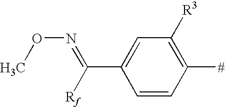 | H | — |
| I-2238 | Q.213 | H | F |
| I-2239 | Q.213 | H | Cl |
| I-2240 | Q.213 | H | Br |
| I-2241 | Q.213 | H | OCH₃ |
| I-2242 | Q.213 | H | CF₃ |
| I-2243 | Q.213 | H | OCF₃ |
| I-2244 | Q.213 | CH₃ | — |
| I-2245 | Q.213 | CH₃ | F |
| I-2246 | Q.213 | CH₃ | Cl |
| I-2247 | Q.213 | CH₃ | Br |
| I-2248 | Q.213 | CH₃ | OCH₃ |
| I-2249 | Q.213 | CH₃ | CF₃ |
| I-2250 | Q.213 | CH₃ | OCF₃ |
| I-2251 | Q.213 | CH₂CH₃ | — |
| I-2252 | Q.213 | CH₂CH₃ | F |
| I-2253 | Q.213 | CH₂CH₃ | Cl |
| I-2254 | Q.213 | CH₂CH₃ | Br |
| I-2255 | Q.213 | CH₂CH₃ | OCH₃ |
| I-2256 | Q.213 | CH₂CH₃ | CF₃ |
| I-2257 | Q.213 | CH₂CH₃ | OCF₃ |
| I-2258 | Q.213 | NHC(═O)CH₃ | — |
| I-2259 | Q.213 | NHC(═O)CH₃ | F |
| I-2260 | Q.213 | NHC(═O)CH₃ | Cl |
| I-2261 | Q.213 | NHC(═O)CH₃ | Br |
| I-2262 | Q.213 | NHC(═O)CH₃ | OCH₃ |
| I-2263 | Q.213 | NHC(═O)CH₃ | CF₃ |
| I-2264 | Q.213 | NHC(═O)CH₃ | OCF₃ |
| I-2265 | Q.213 | NHC(═O)CF₃ | — |
| I-2266 | Q.213 | NHC(═O)CF₃ | F |
| I-2267 | Q.213 | NHC(═O)CF₃ | Cl |
| I-2268 | Q.213 | NHC(═O)CF₃ | Br |
| I-2269 | Q.213 | NHC(═O)CF₃ | OCH₃ |
| I-2270 | Q.213 | NHC(═O)CF₃ | CF₃ |
| I-2271 | Q.213 | NHC(═O)CF₃ | OCF₃ |
| I-2272 | 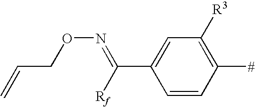 Q.214 | H | — |
| I-2273 | Q.214 | H | F |
| I-2274 | Q.214 | H | Cl |
| I-2275 | Q.214 | H | Br |
| I-2276 | Q.214 | H | OCH₃ |
| I-2277 | Q.214 | H | CF₃ |
| I-2278 | Q.214 | H | OCF₃ |
| I-2279 | Q.214 | CH₃ | — |
| I-2280 | Q.214 | CH₃ | F |
| I-2281 | Q.214 | CH₃ | Cl |
| I-2282 | Q.214 | CH₃ | Br |
| I-2283 | Q.214 | CH₃ | OCH₃ |
| I-2284 | Q.214 | CH₃ | CF₃ |
| I-2285 | Q.214 | CH₃ | OCF₃ |
| I-2286 | Q.214 | CH₂CH₃ | — |
| I-2287 | Q.214 | CH₂CH₃ | F |
| I-2288 | Q.214 | CH₂CH₃ | Cl |
| I-2289 | Q.214 | CH₂CH₃ | Br |
| I-2290 | Q.214 | CH₂CH₃ | OCH₃ |
| I-2291 | Q.214 | CH₂CH₃ | CF₃ |
| I-2292 | Q.214 | CH₂CH₃ | OCF₃ |
| I-2293 | Q.214 | NHC(═O)CH₃ | — |
| I-2294 | Q.214 | NHC(═O)CH₃ | F |
| I-2295 | Q.214 | NHC(═O)CH₃ | Cl |
| I-2296 | Q.214 | NHC(═O)CH₃ | Br |
| I-2297 | Q.214 | NHC(═O)CH₃ | OCH₃ |
| I-2298 | Q.214 | NHC(═O)CH₃ | CF₃ |
| I-2299 | Q.214 | NHC(═O)CH₃ | OCF₃ |
| I-2300 | Q.214 | NHC(═O)CF₃ | — |
| I-2301 | Q.214 | NHC(═O)CF₃ | F |
| I-2302 | Q.214 | NHC(═O)CF₃ | Cl |
| I-2303 | Q.214 | NHC(═O)CF₃ | Br |
| I-2304 | Q.214 | NHC(═O)CF₃ | OCH₃ |
| I-2305 | Q.214 | NHC(═O)CF₃ | CF₃ |
| I-2306 | Q.214 | NHC(═O)CF₃ | OCF₃ |
| I-2307 | 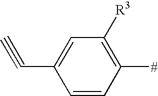 Q.215 | — | — |
| I-2308 | Q.215 | — | F |
| I-2309 | Q.215 | — | Cl |
| I-2310 | Q.215 | — | Br |
| I-2311 | Q.215 | — | OCH₃ |
| I-2312 | Q.215 | — | CF₃ |
| I-2313 | Q.215 | — | OCF₃ |
| I-2314 | 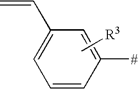 Q.216 | — | — |
| I-2315 | Q.216 | — | 2-F |
| I-2316 | Q.216 | — | 2-Cl |
| I-2317 | Q.216 | — | 2-Br |
| I-2318 | Q.216 | — | 2-OCH₃ |
| I-2319 | Q.216 | — | 2-CF₃ |
| I-2320 | Q.216 | — | 2-OCF₃ |
| I-2321 | Q.216 | — | — |
| I-2322 | Q.216 | — | 4-F |
| I-2323 | Q.216 | — | 4-Cl |
| I-2324 | Q.216 | — | 4-Br |
| I-2325 | Q.216 | — | 4-OCH₃ |
| I-2326 | Q.216 | — | 4-CF₃ |
| I-2327 | Q.216 | — | 4-OCF₃ |
| I-2328 | 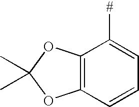 Q.217 | — | — |
| I-2329 | 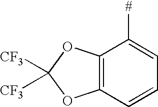 Q.219 | — | — |
| I-2330 | 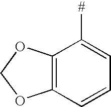 Q.220 | — | — |
| I-2331 | 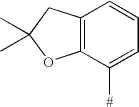 Q.221 | — | — |
| I-2332 | 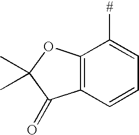 Q.222 | — | — |
| I-2333 | 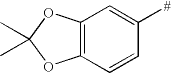 Q.223 | — | — |
| I-2334 | 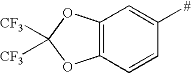 Q.224 | — | — |
| I-2335 | 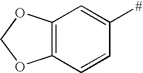 Q.225 | — | — |
| I-2336 | 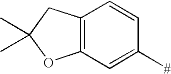 Q.226 | — | — |
| I-2337 | 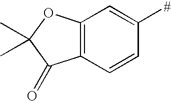 Q.227 | — | — |

The compounds of the formula I are especially suitable for efficiently combating the following pests:

insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis,
beetles (Coleoptera), for example Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus napi, Chaetocnema tibialis, Conoderus vespertinus, Crioceris asparagi, Ctenicera ssp., Diabrotica longicornis, Diabrotica semipunctata, Diabrotica 12-punctata Diabrotica speciosa, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popillia japonica, Sitona lineatus and Sitophilus granaria,
flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilia cuprina, Lucilia sericata, Lycoria pectoralis, Mansonia titillanus, Mayetiola destructor, Musca domestica, Muscina stabulans, Oestrus ovis, Opomyza florum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga sp., Simulium vittatum, Stomoxys calcitrans, Tabanus bovinus, Tabanus atratus, Tabanus lineola, and Tabanus similis, Tipula oleracea, and Tipula paludosa
thrips (Thysanoptera), e.g. Dichromothrips corbetti, Dichromothrips ssp, Frankliniella fusca, Frankliniella occidentalis, Frankliniella tritici, Scirtothrips citri, Thrips oryzae, Thrips palmi and Thrips tabaci,
termites (Isoptera), e.g. Calotermes flavicollis, Leucotermes flavipes, Heterotermes aureus, Reticulitermes flavipes, Reticulitermes virginicus, Reticulitermes lucifugus, Termes natalensis, and Coptotermes formosanus,
cockroaches (Blattaria—Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,
true bugs (Hemiptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis, Thyanta perditor, Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus vitifolii, Cimex lectularius, Cimex hemipterus, Reduvius senilis, Triatoma spp., and Arilus critatus.
ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Monomorium pharaonis, Solenopsis geminata, Solenopsis invicta, Solenopsis richteri, Solenopsis xyloni, Pogonomyrmex barbatus, Pogonomyrmex californicus, Pheidole megacephala, Dasymutilla occidentalis, Bombus spp. Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile,
crickets, grasshoppers, locusts (Orthoptera), e.g. Acheta domestica, Gryllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina,
Arachnoidea, such as arachnids (Acarina), e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabilis, Hyalomma truncatum, Ixodes ricinus, Ixodes rubicundus, Ixodes scapularis, Ixodes holocyclus, Ixodes pacificus, Ornithodorus moubata, Ornithodorus hermsi, Ornithodorus turicata, Ornithonyssus bacoti, Otobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis; Araneida, e.g. Latrodectus mactans, and Loxosceles reclusa,
fleas (Siphonaptera), e.g. Ctenocephalides felis, Ctenocephalides canis, Xenopsylla cheopis, Pulex irritans, Tunga penetrans, and Nosopsyllus fasciatus,
silverfish, firebrat (Thysanura), e.g. Lepisma saccharina and Thermobia domestica,
centipedes (Chilopoda), e.g. Scutigera coleoptrata,
millipedes (Diplopoda), e.g. Narceus spp.,
Earwigs (Dermaptera), e.g. forficula auricularia,
lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus,

Plant parasitic nematodes such as root-knot nematodes, Meloidogyne arenaria, Meloidogyne chitwoodi, Meloidogyne exigua, Meloidogyne hapla, Meloidogyne incognita, Meloidogyne javanica and other Meloidogyne species; cyst nematodes, Globodera rostochiensis, Globodera pallida, Globodera tabacum and other Globodera species, Heterodera avenae, Heterodera glycines, Heterodera schachtii, Heterodera trifolii, and other Heterodera species; seed gall nematodes, Anguina funesta, Anguina tritici and other Anguina species; stem and foliar nematodes, Aphelenchoides besseyi, Aphelenchoides fragariae, Aphelenchoides ritzemabosi and other Aphelenchoides species; sting nematodes, Belonolaimus longicaudatus and other Belonolaimus species; pine nematodes, Bursaphelenchus xylophilus and other Bursaphelenchus species; ring nematodes, Criconema species, Criconemella species, Criconemoides species, and Mesocriconema species; stem and bulb nematodes, Ditylenchus destructor, Ditylenchus dipsaci, Ditylenchus myceliophagus and other Ditylenchus species; awl nematodes, Dolichodorus species; spiral nematodes, Helicotylenchus dihystera, Helicotylenchus multicinctus and other Helicotylenchus species, Rotylenchus robustus and other Rotylenchus species; sheath nematodes, Hemicycliophora species and Hemicriconemoides species; Hirshmanniella species; lance nematodes, Hoplolaimus columbus, Hoplolaimus galeatus and other Hoplolaimus species; false root-knot nematodes, Nacobbus aberrans and other Nacobbus species; needle nematodes, Longidorus elongates and other Longidorus species; pin nematodes, Paratylenchus species; lesion nematodes, Pratylenchus brachyurus, Pratylenchus coffeae, Pratylenchus curvitatus, Pratylenchus goodeyi, Pratylencus neglectus, Pratylenchus penetrans, Pratylenchus scribneri, Pratylenchus vulnus, Pratylenchus zeae and other Pratylenchus species; Radinaphelenchus cocophilus and other Radinaphelenchus species; burrowing nematodes, Radopholus similis and other Radopholus species; reniform nematodes, Rotylenchulus reniformis and other Rotylenchulus species; Scutellonema species; stubby root nematodes, Trichodorus primitivus and other Trichodorus species; Paratrichodorus minor and other Paratrichodorus species; stunt nematodes, Tylenchorhynchus claytoni, Tylenchorhynchus dubius and other Tylenchorhynchus species and Merlinius species; citrus nematodes, Tylenchulus semipenetrans and other Tylenchulus species; dagger nematodes, Xiphinema americanum, Xiphinema index, Xiphinema diversicaudatum and other Xiphinema species; and other plant parasitic nematode species.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7515-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and binders.

Examples of suitable solvents are water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.

Examples of suitable carriers are ground natural minerals (for example kaolins, clays, talc, chalk) and ground synthetic minerals (for example highly disperse silica, silicates).

Suitable emulsifiers are nonionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants used are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone or water.

Also anti-freezing agents such as glycerin, ethylene glycol, propylene glycol and bactericides such as can be added to the formulation.

Suitable antifoaming agents are for example antifoaming agents based on silicon or magnesium stearate.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound(s). In this case, the active compound(s) are employed in a purity of from 90% to 100% by weight, preferably 95% to 100% by weight (according to NMR spectrum).

The compounds of formula I can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compound(s) according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1% per weight.

The active compound(s) may also be used successfully in the ultra-low-volume process (ULV), it being possible to apply formulations comprising over 95% by weight of active compound, or even to apply the active compound without additives.

The following are examples of formulations: 1. Products for dilution with water for foliar applications. For seed treatment purposes, such products may be applied to the seed diluted or undiluted.

### A) Water-Soluble Concentrates (SL, LS)

10 parts by weight of the active compound(s) are dissolved in 90 parts by weight of water or a water-soluble solvent. As an alternative, wetters or other auxiliaries are added. The active compound(s) dissolves upon dilution with water, whereby a formulation with 10% (w/w) of active compound(s) is obtained.

### B) Dispersible Concentrates (DC)

20 parts by weight of the active compound(s) are dissolved in 75 parts by weight of cyclohexanone with addition of 10 parts by weight of a dispersant, for example polyvinylpyrrolidone. Dilution with water gives a dispersion, whereby a formulation with 20% (w/w) of active compound(s) is obtained.

### C) Emulsifiable Concentrates (EC)

15 parts by weight of the active compound(s) are dissolved in 75 parts by weight of xylene with addition of calcium dodecylbenzenesulfonate and castor oil ethoxylate (in each case 5 parts by weight). Dilution with water gives an emulsion, whereby a formulation with 15% (w/w) of active compound(s) is obtained.

### D) Emulsions (EW, EO, ES)

40 parts by weight of the active compound(s) are dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound(s) is obtained.

### E) Suspensions (SC, OD, FS)

In an agitated ball mill, 20 parts by weight of the active compound(s) are comminuted with addition of 10 parts by weight of dispersants, wetters and 70 parts by weight of water or of an organic solvent to give a fine active compound(s) suspension. Dilution with water gives a stable suspension of the active compound(s), whereby a formulation with 20% (w/w) of active compound(s) is obtained.

### F) Water-Dispersible Granules and Water-Soluble Granules (WG, SG)

50 parts by weight of the active compound(s) are ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound(s), whereby a formulation with 50% (w/w) of active compound(s) is obtained.

### G) Water-Dispersible Powders and Water-Soluble Powders (WP, SP, SS, WS)

75 parts by weight of the active compound(s) are ground in a rotor-stator mill with addition of 25 parts by weight of dispersants, wetters and silica gel. Dilution with water gives a stable dispersion or solution of the active compound(s), whereby a formulation with 75% (w/w) of active compound(s) is obtained.

2. Products to be applied undiluted for foliar applications. For seed treatment purposes, such products may be applied to the seed diluted or undiluted.

### H) Dustable Powders (DP, DS)

5 parts by weight of the active compound(s) are ground finely and mixed intimately with 95 parts by weight of finely divided kaolin. This gives a dustable product having 5% (w/w) of active compound(s)

### I) Granules (GR, FG, GG, MG)

0.5 part by weight of the active compound(s) is ground finely and associated with 95.5 parts by weight of carriers, whereby a formulation with 0.5% (w/w) of active compound(s) is obtained. Current methods are extrusion, spray-drying or the fluidized bed. This gives granules to be applied undiluted for foliar use.

J) ULV solutions (UL, LS)

10 parts by weight of the active compound(s) are dissolved in 90 parts by weight of an organic solvent, for example xylene. This gives a product having 10% (w/w) of active compound(s), which is applied undiluted for foliar use.

Various types of oils, wetters, adjuvants, herbicides, fungicides, other pesticides, or bactericides may be added to the active ingredients, if appropriate just immediately prior to use (tank mix). These agents usually are admixed with the agents according to the invention in a weight ratio of 1:10 to 10:1.

The compounds of formula I are effective through both contact and ingestion.

The compounds of formula I are also suitable for the protection of the seed, plant propagules and the seedlings' roots and shoots, preferably the seeds, against soil pests and also for the treatment plant seeds which tolerate the action of herbicides or fungicides or insecticides owing to breeding, including genetic engineering methods.

Conventional seed treatment formulations include for example flowable concentrates FS, solutions LS, powders for dry treatment DS, water dispersible powders WS or granules for slurry treatment, water soluble powders SS and emulsion ES. Application to the seeds is carried out before sowing, either directly on the seeds.

The seed treatment application of the compounds of formula I or formulations containing them is carried out by spraying or dusting the seeds before sowing of the plants and before emergence of the plants.

The invention also relates to the propagation product of plants, and especially the treated seed comprising, that is, coated with and/or containing, a compound of formula I or a composition comprising it. The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

The seed comprises the inventive compounds or compositions comprising them in an amount of from 0, 1 g to 10 kg per 100 kg of seed.

Compositions of this invention may also contain other active ingredients, for example other pesticides, insecticides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

The following list of pesticides together with which the compounds according to the invention can be used, is intended to illustrate the possible combinations, but not to impose any limitation:

A.1. Organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
A.2. Carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
A.3. Pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin;
A.4. Growth regulators: a) chitin synthesis inhibitors: benzoylureas: chlorfluazuron, cyramazin, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, a tetronic acid derivative of formula Γ¹,

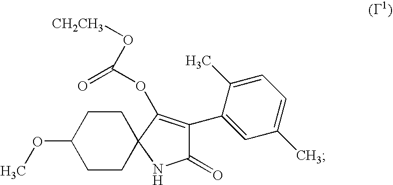

A.5. Nicotinic receptor agonists/antagonists compounds: clothianidin, dinotefuran, imidacloprid, thiamethoxam, nitenpyram, acetamiprid, thiacloprid;
A.6. GABA antagonist compounds: acetoprole, endosulfan, ethiprole, fipronil, vaniliprole;
A.7. Macrocyclic lactone insecticides: abamectin, emamectin, milbemectin, lepimectin, spinosad;
A.8. METI I acaricides: fenazaquin, pyridaben, tebufenpyrad, tolfenpyrad;
A.9. METI II and III compounds: acequinocyl, fluacyprim, hydramethylnon;
A.10. Uncoupler compounds: chlorfenapyr;
A.11. Oxidative phosphorylation inhibitor compounds: cyhexatin, diafenthiuron, fenbutatin oxide, propargite;
A.12. Moulting disruptor compounds: cryomazine;
A.13. Mixed Function Oxidase inhibitor compounds: piperonyl butoxide;
A.14. Sodium channel blocker compounds: indoxacarb, metaflumizone;
A.15. Various: benclothiaz, bifenazate, cartap, flonicamid, pyridalyl, pymetrozine, sulfur, thiocyclam, N—R′-2,2-dihalo-1-R″cyclo-propanecarboxamide-2-(2,6-dichloro-α, α,α-tri-fluoro-p-tolyl)hydrazone or N—R′-2,2-di(R′″)propionamide-2-(2,6-dichloro-α, α,α-trifluoro-p-tolyl)-hydrazone, wherein R′ is methyl or ethyl, halo is chloro or bromo, R″ is hydrogen or methyl and R′″ is methyl or ethyl, and the aminoisothiazole compounds of formula Γ².

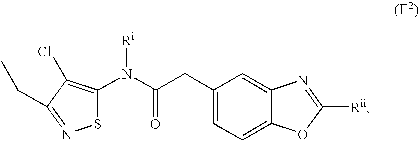

wherein Rⁱ is —CH₂OCH₂CH₃ or H and Rⁱⁱ is CF₂CF₂CF₃ or CH₂CH(CH₃)₃, anthranilamide compounds of formula Γ¹

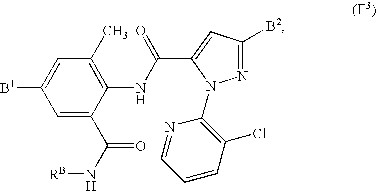

wherein B¹ is hydrogen, chlorine or cyano, B² is a bromine atom or CF₃, and R^{B} is H, CH₃ or CH(CH₃)₂, and malononitrile compounds as described in JP 2002 284608, WO 02/89579, WO 02/90320, WO 02/90321, WO 04/06677, WO 04/20399, or JP 2004 99597.

The insects may be controlled by contacting the target parasite/pest, its food supply, habitat, breeding ground or its locus with a pesticidally effective amount of compounds of or compositions of formula I.

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds or compositions of the invention can also be applied preventively to places at which occurrence of the pests is expected.

The compounds of formula I may also be used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

In the case of soil treatment or of application to the pests dwelling place or nest, the quantity of active ingredient ranges from 0.0001 to 500 g per 100 m², preferably from 0.001 to 20 g per 100 m².

For use in treating crop plants, the rate of application of the active ingredients of this invention may be in the range of 0.1 g to 4000 g per hectare, desirably from 25 g to 600 g per hectare, more desirably from 50 g to 500 g per hectare.

Compounds of formula I and compositions comprising them can also be used for controlling and preventing infestations and infections in animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula I and compositions comprising them are suitable for systemic and/or non-systemic control of ecto- and/or endoparasites. They are active against all or some stages of development.

Administration can be carried out both prophylactically and therapeutically. Administration of the active compounds is carried out directly or in the form of suitable preparations, orally, topically/dermally or parenterally.

For oral administration to warm-blooded animals, the formula I compounds may be formulated as animal feeds, animal feed premixes, animal feed concentrates, pills, solutions, pastes, suspensions, drenches, gels, tablets, boluses and capsules. In addition, the formula I compounds may be administered to the animals in their drinking water. For oral administration, the dosage form chosen should provide the animal with 0.01 mg/kg to 100 mg/kg of animal body weight per day of the formula I compound, preferably with 0.5 mg/kg to 100 mg/kg of animal body weight per day.

Alternatively, the formula I compounds may be administered to animals parenterally, for example, by intraruminal, intramuscular, intravenous or subcutaneous injection. The formula I compounds may be dispersed or dissolved in a physiologically acceptable carrier for subcutaneous injection. Alternatively, the formula I compounds may be formulated into an implant for subcutaneous administration. In addition the formula I compound may be transdermally administered to animals. For parenteral administration, the dosage form chosen should provide the animal with 0.01 mg/kg to 100 mg/kg of animal body weight per day of the formula I compound.

The formula I compounds may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the formula I compound. In addition, the formula I compounds may be formulated as ear tags for animals, particularly quadrupeds such as cattle and sheep.

Suitable preparations are:
Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Generally it is favorable to apply solid formulations which release compounds of formula I in total amounts of 10 mg/kg to 300 mg/kg, preferably 20 mg/kg to 200 mg/kg. The active compounds can also be used as a mixture with synergists or with other active compounds which act against pathogenic endo- and ectoparasites.

In general, the compounds of formula I are applied in parasiticidally effective amount meaning the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

### SYNTHESIS EXAMPLES

With due modification of the starting compounds, the protocols shown in the synthesis example below were used for obtaining further compounds I. The resulting compounds, together with physical data, are listed in the table 1 which follows.

The products were characterized by coupled High Performance Liquid Chromatography/mass spectrometry (HPLC/MS), by ¹H-NMR (400 MHz) in CDCl₃ or by their melting points. HPLC column: RP-18 column (Chromolith Speed ROD from Merck KgaA, Germany). Elution: acetonitrile+0.1% trifluoroacetic acid (TFA)/water in a ratio of from 5:95 to 95:5 in 5 minutes at 40° C. MS: Quadrupol electrospray ionisation, 80 V (positiv modus).

### Example 1

### 2-{3-[3-Chloro-5-(3,3-dichloro-allyloxy)-phenoxy]-propyloxy}-5-trifluoro methyl-pyridine (I.1)

Step 1: 1-(3-Bromo-propyloxy-3-chloro-5-methoxybenzene (VI.1)

12.7 g (63 mmol) 1,3-Dibromopropane, 6.53 g (47.3 mmol) K₂CO₃ and 5.0 g (31.5 mmol) 3-chloro-5-methoxyphenol in 150 ml dimethylformamide were heated for 12 hours to 60° C. and stirred at 20-25° C. for 2 days. The solvent was removed in vacuo, water was added and the product extracted with ethyl acetate. Removal of the solvent and column chromatographie afforded 7.1 g of approx 80% pure product.

¹H-NMR [ppm]: 6.5 (3, 2H), 6.2 (s, 1H), 4.1 (m, 2H), 3.8 (s, 3H), 3.5 (m, 2H), 2.2-2.1 (m, 2H).

Step 2: 1-(3-Bromo-propyloxy-3-chloro-5-methoxybenzene (X.1) via benzoic acid-3-(3-chloro-5-methoxyphenoxy)-propylester (IX.1)

7.09 g (21 mmol) of crude product (VI.1), 2.9 g (24.1 mmol) benzoic acid and 4.54 g (33 mmol) of K₂CO₃ were stirred in 100 ml dimethylformaide at 50° C. for 6 hours, then at 20-25° C. for 2 days. The solvent was removed in vacuo, water was added and the product extracted with ethyl acetate. Removal of the solvent and column

chromatographie afforded 6.4 g of the benzoic acid ester compound (IX.1) as an intermediate pure enough for further transformation. (¹H-NMR [ppm]: 8.0 (m, 2H), 7.6 (m, 1H), 7.4 (m, 2H), 6.5 (s, 1H), 6.3 (m, 1H), 4.5 (m, 2H), 4.1 (m, 2H), 3.7 (s, 3H), 2.3 (m, 2H)).

1.5 g (26 mmol) of KOH in 50 ml methanol and 8 ml of water were added and the mixture was stirred at 20-25° C. for 16 hours. 300 ml of ether and 200 ml water were added and the organic layer was washed with 100 ml water and 100 ml sat. K₂CO₃. Evaporation and column chromatographie afforded 2 g of the title compound (X.1). ¹H-NMR [ppm]: 6.5 (s, 2H), 6.3 (s, 1H), 4.1 (m, 2H), 3.8 (m, 2H), 3.5 (s, 3H), 2.0 (m, 2H).

Step 3: 2-[3-(-3-Chloro-5-methoxyphenoxy)-propyloxy]-5-trifluormethylpyridine (IV.1)

To 0.20 g of 95% NaH in 50 ml DMF, 1.5 g (6.9 mmol) of compound (X.1) in 50 ml of DMF was added dropwise. After stirring for 1 hour at 20-25° C., 1.253 g 2-chloro-5-trifloromethylpyridine is added in 30 ml of DMF and the reaction mixture was stirred at 100° C. for 6 hours and then at 20-25° C. for 16 hours. After addition of water and extraction with ethyl acetat, 2.1 g of crude product (IV.1) were obtained. ¹H-NMR [ppm]: 8.4 (m, 1H), 7.8 (ps-d, 1H), 6.8 (m, 1H), 6.5 (m, 2H), 6.3 (m, 1H), 4.5 (m, 2H), 4.1 (m, 2H), 3.8 (s, 3H), 2.2 (m, 2H).

Step 4: 3-Chloro-5-[3-(5-trifluormethylpyridine-2-yloxy)-propyloxy]phenol (II.1)

2.0 g (5.5 mmol) of compound (IV.1) in 20 ml CH₂Cl₂ were cooled to 0° C. and treated with 16.5 ml (16.5 mmol) of an 1 M solution of BBr₃ in CH₂Cl₂. The reaction mixture was stirred at 20-25° C. for 10 hours, water was added, and the mixture was washed with water to yield 0.54 g of the product (II.1). NMR (d⁶-DMSO) [ppm]: 8.6 (m, 1H), 8.0 ((m, 1H), 7.0 (m, 1H), 6.5-6.4 (m, 2H), 6.3 (m, 1H), 4.4 (m, 2H), 4.0 (m, 2H), 2.0 (m, 2H).

Step 5: 2-{3-[3-Chloro-5-(3,3-dichloro-allyloxy)-phenoxy]-propyloxy}-5-trifluoro methyl-pyridine (I.1)

To 0.74 g (2.1 mmol) of compound (II.1) in 60 ml DMF, 0.44 g (3.2 mmol) of K₂CO₃ and 0.373 g (2.3 mmol) of 1,3,3-trichloropropene were added and stirred at 90° C. for 3 hours and at 20-25° C. for 16 hours. The solvent was removed and the remaining crude product subjected to column chromatography affording 0.43 g of the title compound (1.1) as a yellow oil.

¹H-NMR [ppm]: 8.4 (m 1H), 7.8 (m, 1H), 6.8 (m, 1H), 6.6-6.5 (m, 2H), 6.2 (m 1H), 6.1 (t, 1H), 4.6-4.5 (m, 4H), 4.1 (mc, 2H), 2.1 (m, 4H).

### Example 2

### 2-{6-[3-chloro-5-(3,3-dichloro-allyloxy)-phenoxy]-hexyloxy}-5-trifluoromethylpyridine (I.2)

Steps 1 to 3 are conducted in analogy to the steps 1 to 3 of example 1 above.

Step 4: 2-{6-[3-chloro-5-hydroxy-phenoxy]-hexyloxy}-5-trifluoromethylpyridine (II.2)

1.10 g (2.7 mmol) 5-chloro-2-[6-(3-chloro-5-methoxy-phenoxyhexyloxy)]-5-trifluormethylpyridine in 20 ml CH₂Cl₂ were cooled to 0° C. and treated with 3.2 ml (3.2 mmol) of an 1 M solution of BBr₃ in CH₂Cl₂. The reaction mixture was stirred at 20-25° C. for 10 hours, water was added, and the mixture was washed with water to yield 0.54 g of compound (II.2).

¹H-NMR [ppm]: 8.4 (m, 1H), (m, 1H), 6.8 (m, 2H), 6.2 (m, 1H), 4.4 (m, 2H), 3.8 (m, 2H), 1.9-1.6 (m, 4H), 1.5 (m, 4H).

Step 5: 2-{6-[3-chloro-5-(3,3-dichloro-allyloxy)-phenoxy]-hexyloxy}-5-trifluoromethylpyridine (I.2)

To 0.53 g (1.4 mmol) of compound (II.2) in 60 ml DMF, 0.29 g (2.1 mmol) of K₂CO₃ and 0.242 g (1.5 mmol) of 1,3,3-trichloropropene were added and stirred at 90° C. for 3 hours and at 20-25° C. for 16 hours. The solvent was removed and the remaining crude product subjected to column chromatography affording 0.4 g of the title compound as a yellow oil.

¹H-NMR [ppm]: 8.4 (m, 1H), 7.7 (m, 1H), 6.8 (m, 1H), 6.6-6.5 (m, 2H), 6.3 (m, 1H), 6.1 (t, 1H), 4.6 (d, 2H), 4.3 (mc, 2H), 3.9 (mc, 2H), 1.9-1.7 (m, 2H), 1.6-1.5 (m, 4H).

TABLE I

| | |
|---|---|
| | (I) |
| 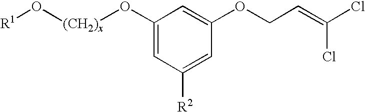 | |

| | | | | Physical data |
|---|---|---|---|---|
| | | | | ¹H-NMR (CDCl₃, δ [ppm]); HPLC/MS (RT [min]; m/z |
| No. | R¹ | R² | x | [M + H]⁺; mp. [° C.] |
| I.1 | 2-(5-CF₃)-pyridyl | Cl | 3 | 8.4 (m), 7.8 (m), 6.8 (m), 6.6-6.5 (m), 6.2 (m), 6.1 (t), |
| | | | | 4.6-4.5 (m), 4.1 (mc), 2.1 (m). |
| I.2 | 2-(5-CF₃)-pyridyl | Cl | 6 | 8.4 (m), 7.7 (m), 6.8 (m), 6.6-6.5 (m), 6.3 (m), 6.1 (t), 4.6 |
| | | | | (d), 4.3 (mc), 3.9 (mc), 1.9-1.7 (m), 1.6-1.5 (m). |
| I.3 | 2-(5-CF₃)-pyridyl | Cl | 4 | 8.4 (m), 7.7 (m), 6.8 (m), 6.6-6.5 (m), 6.3 (m), 6.1 (t), 4.6 |
| | | | | (d), 4.4 (m), 4.0 (m), 2.0-1.9 (m). |
| I.4 | 2-(5-CF₃)-pyridyl | Cl | 7 | 8.4 (m), 7.8 (m), 6.8 (m), 6.6-6.4 (m), 6.3 (m), 6.1 t, 4.6 d, |
| | | | | 4.4 (m), 3.9 (m), 1.8 (m), 1.5 (m). |
| I.5 | 2-(4-CH₃)-thiazole | Cl | 6 | 6.6-6.4 (m), 6.3 s, 6.2 s, 6.1 t, 4.6 d, 4.4 (m), 3.9 (m), 2.2 |
| | | | | s, 1.9-1.8 (m), 1.6-1.5 (m). |
| I.6 | 2-(4-CF₃)-pyridyl | Cl | 6 | 8.3 (m), 7.1 d, 6.9 s, 6.5-6.4 (m), 6.3 (m), 6.1 t, 4.6 d, 4.4 |
| | | | | (m), 4.0 (m), 1.9-1.8 (m), 1.6-1.5 (m) (m), 1.9-1.8 (m), |
| | | | | 1.6-1.5 (m). |
| I.7 | 2-(4-CH₃-thiazole | Cl | 5 | 6.6-6.4 (m), 6.3 (m), 6.2 (m), 6.1 t, 4.6 d, 4.4 (m), 3.9 (m), |
| | | | | 2.2 s, 2.0-1.8 (m), 1.7-1.6 (m). |
| 1.8 | 2-(3-CF₃)-pyridyl | Cl | 5 | 8.2 (m), 7.8 (m), 7.0 (m), 6.6-6.4 (m), 6.3 s, 6.1 t, 4.6 d, |
| | | | | 4.4 (m), 3.9 (m), 1.9-1.8 (m), 1.7-1.6 (m) 8.2 (m), 7.8 (m). |
| | | | | 7.0 (m), 6.6-6.4 (m), 6.3 s, 6.1 t, 4.6 d, 4.4 (m), 3.9 (m), |
| | | | | 1.9-1.8 (m), 1.7-1.6 (m). |
| I.9 | 2-(5-CH₃S(═O)₂)-pyridyl | Cl | 4 | 8.7 d, 8.1-7.9 (m), 6.9-6.8 (m), 6.3 (m), 6.1 (t), 4.6 (d), 4.5 |
| | | | | (m), 4.0 (m), 3.0 (s), 2.0-1.9 (m). |
| I.10 | 2-(5-CF₃)-pyridyl | Cl | 2 | 8.4 (s, 7.8 (m), 6.9-6.8 (m), 6.66.5 (m), 6.4 (s), 6.1 (t), 4.7 |
| | | | | (m), 4.6 d, 4.3 (m). |
| I.11 | 2-(3-CF₃)-pyridyl | Cl | 4 | 8.3 (m), 7.9-7.8 (m), 7.0-6.8 (m), 6.6-6.4 (m), 6.3 (m), 6.1 |
| | | | | (t), 4.6 (d), 4.5 (m), 4.0 (m), 2.1-1.9 (m). |
| I.12 | 2-(4-CF₃)-pyridyl | Cl | 4 | 8.3 (m), 7.1-7.0 (m), 6.9 s, 6.6-6.4 (m), 6.3 (m), 6.1 (t), |
| | | | | 4.6 (d), 4.4 (m), 4.0 (m), 2.0-1.9 (m). |
| I.13 | 2,2,-dimethyl-2,3- | Cl | 4 | 6.8 (m), 6.5-6.4 (m), 6.3 (m), 6.1 (t), 4.6 (d), 4.2 (m), 4.0 |
| | dihydrobenzo[b]furan-7-yl | | | (m), 3.0 (s), 2.1-1.9 (m). |
| I.14 | 2-(5-CF₃)-pyridyl | Cl | 5 | 8.4 (m), 7.7 (d), 6.7 (m), 6.5-6.4 (m), 6.3 (m), 6.1 (t), 4.5 |
| | | | | (d), 4.3 (m), 3.8 (m), 1.9-1.8 (m), 1.6-1.5 (m). |
| I.15 | 2-(4-CF₃)-pyridyl | Cl | 5 | 8.3 (d), 7.2 (m), 7.0 (s), 6.6-6.4 (m), 6.3 (m), 6.1 (t), 4.6 |
| | | | | (d), 4.4 (m), 4.0 (m), 1.9-1.8 (m), 1.7-1.6 (m). |
| I.16 | 2-(3-CF₃)-pyridyl | Cl | 6 | 8.3 (m), 7.8 (d), 6.9 (d), 6.6-6.4 (m), 6.3 (m), 6.1 (t), 4.6 |
| | | | | (d), 4.4 (m), 3.9 (m), 1.9-1.8 (m), 1.6-1.5 (m). |
| I.17 | 2-(3-CF₃)-pyridyl | Cl | 3 | 8.3 (m), 7.8 (d), 6.9 (d), 6.6-6.4 (m), 6.3 (m), 6.1 (t), 4.6 |
| | | | | (d), 4.6 (m), 4.1 (m), 2.3-2.2 (m). |
| I.18 | 2-(4-CF₃)-pyridyl | Cl | 3 | 8.3 (m), 7.1 (m), 7.0 (s), 6.6-6.4 (m), 6.3 (m), 6.1 (t), 4.6 |
| | | | | (d), 4.4 (m), 4.2 (m), 2.3-2.2 (m). |
| I.19 | 2-(5-CH₃S(═O)₂)-pyridyl | Cl | 3 | 8.8 (s), 8.0 (pss), 6.9-6.8 (m), 6.5-6.4 (m), 6.3 (s), 6.1 (t), |
| | | | | 4.6 (d), 4.6 (m), 3.0 (s), 2.3-2.2 (m). |

Examples for the Action Against Harmful Pests

1. Activity Against Boll Weevil (Anthonomus grandis)

The active compounds were formulated in 1:3 DMSO:water. 10 to 15 eggs were placed into microtiterplates filled with 2% agar-agar in water and 300 ppm formaline. The eggs were sprayed with 20 μl of the test solution, the plates were sealed with pierced foils and kept at 24-26° C. and 75-85% humidity with a day/night cycle for 3 to 5 days. Mortality was assessed on the basis of the remaining unhatched eggs or larvae on the agar surface and/or quantity and depth of the digging channels caused by the hatched larvae. Tests were replicated 2 times.

In this test, compounds I.1, I.2 and I.3 at 2500 ppm showed over 75% mortality.

2. Activity Against Tobacco Budworm (Heliothis virescens)

The active compounds were formulated in 1:3 DMSO:water. 15 to 25 eggs were placed into microtiterplates filled with diet. The eggs were sprayed with 10 μl of the test solution, the plates were sealed with pierced foils and kept at 27-29° C. and 75-85% humidity under fluorescent light for 6 days. Mortality was assessed on the basis of the agility and of comparative feeding of the hatched larvae. Tests were replicated 2 times.

In this test, compounds I.1, I.2 and I.3 at 2500 ppm showed over 75% mortality.

### 3. Activity Against Southern Armyworm (Spodoptera Eridania), 2Nd Instar Larvae

The active compounds were formulated for testing the activity against insects and arachnids as a 10.000 ppm solution in a mixture of 35% acetone and water, which was diluted with water, if needed.

A Sieva lima bean leaf was dipped in the test solution and allowed to dry. The leaf was then placed in a plastic perforated zip enclosure bag and ten 2nd instar larvae were added. At 4 days, observations are made of mortality and reduced feeding.

In this test, the compounds I.1, I.2, I.3, I.10, I.11, I.12, I.14, I.15, I.16, I.17, and I.18 at 300 ppm showed a mortality of at least 80% in comparison with untreated controls.

4. Activity Against Argentine Ant (Linepithema Humile), Harvester Ant (Pogonomyrmex californicus), acrobat ant (Crematogaster spp.), carpenter ant (Camponotus floridanus), fire ant (Solenopsis invicta), house fly (Musca domestica), stable fly (Stomoxys calcitrans), flesh fly (Sarcophaga sp.), yellowfever mosquito (Aedes aegyptii), house mosquito (Culex quinquefasciatus), malaria mosquito (Anopheles albimanus), German cockroach (Blattella Germanica), cat flea (Ctenocephalides felis), and brown dog tick (Rhipicephalus sanguineus) via glass contact

Glass vials are treated with 0.5 ml of a solution of active ingredient in acetone and allowed to dry. Insects or ticks are placed into each vial together with some food and moisture supply. The vials are kept at 22° C. and are observed for treatment effects at various time intervals.

5. Activity against yellowfever mosquito (Aedes aegyptii), house mosquito (Culex quinquefasciatus) and malaria mosquito (Anopheles albimanus) larvae via water treatment

Well plates are used as test arenas. The active ingredient is dissolved in acetone and diluted with water to obtain the concentrations needed. The final solutions containing appr. 1% acetone are placed into each well. Approximately 10 mosquito larvae (4^{th}-instars) in 1 ml water are added to each well. Larvae are fed one drop of liver powder each day. The dishes are covered and maintained at 22° C. Mortality is recorded daily and dead larvae and live or dead pupae are removed daily. At the end of the test remaining live larvae are recorded and percent mortality is calculated.

6. Activity against diamond back moth (plutella xylostella)

The active compounds were formulated in 50:50 acetone:water and 0.1% (vol/vol) Alkamuls EL 620 surfactant. A 6 cm leaf disk of cabbage leaves was dipped in the test solution for 3 seconds and allowed to air dry in a Petri plate lined with moist filter paper. The leaf disk was inoculated with 10 third instar larvae and kept at 25-270° C. and 50-60% humidity for 3 days. Mortality was assessed after 72 h of treatment.

In this test, the compounds I.1, I.2, I.10, I.14, and I.15, at 300 ppm showed a mortality of at least 75% in comparison with untreated controls.

1-16. (canceled)

17. A compound of formula I 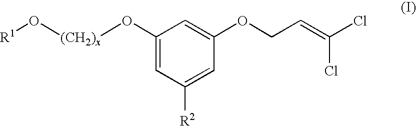
wherein
R¹ is pyridyl, which is substituted by any combination of 1 to 4 groups R³;
R³ is halogen, cyano, nitro, hydroxy, mercapto, amino, C₁-C₆-alkyl, C₂-C₆-alkenyl, C₂-C₆-alkynyl, C₃-C₆-cycloalkyl, C₃-C₆-cycloalkenyl, C₁-C₆-haloalkyl, C₂-C₆-haloalkenyl, C₂-C₆-haloalkynyl, C₃-C₆-halocycloalkyl, C₃-C₆-halocycloalkenyl, C₁-C₆-alkoxy, C₂-C₆-alkenyloxy, C₃-C₆-alkynyloxy, C₁-C₆-haloalkoxy, C₂-C₆-haloalkenyloxy, C₃-C₆-haloalkynyloxy, C₁-C₆-alkylthio, C₂-C₆-alkenylthio, C₃-C₆-alkynylthio, C₁-C₆-haloalkylthio, C₂-C₆-haloalkenylthio, C₃-C₆-haloalkynylthio C₁-C₆-alkylamino, C₂-C₆-alkenylamino, C₂-C₆-alkynylamino, di(C₁-C₆-alkyl)amino, di(C₂-C₆-alkenyl)amino, di(C₂-C₆-alkynyl)amino, C₁-C₆-alkyl-C₂-C₆-alkenylamino, C₁-C₆-alkyl-C₂-C₆-alkynylamino, C₁-C₆-alkenyl-C₂-C₆-alkynylamino, tri(C₁-C₁₀)alkylsilyl, or
phenyl or a 5- to 7-membered saturated or partially unsaturated heterocyclic ring which may contain 1 to 3 heteroatoms selected from the group consisting of oxygen, sulfur and nitrogen or
a 5- or 6-membered heteraromatic ring system which may contain 1 to 4 heteroatoms selected from the group consisting of oxygen, nitrogen and sulfur,
wherein the phenyl and the heteroaromatic ring may be bonded via an oxygen or a sulfur atom,
wherein each R³ group is unsubstituted or substituted with one or more substituents selected from the group consisting of —R^{a}, —C(=G)R^{a}, —C(=G)OR^{a}, —C(=G)NR^{a}₂, —C(=G)[N═SR^{a}₂], —C(═NOR^{a})R^{a}, —C(═NOR^{a})NR^{a}₂, —C(═NNR^{a}₂)R^{a}, —OC(=G)-OC(=G)OR^{a}, N═SR^{a}₂, —NR^{a}C(=G)R^{a}, —N[C(=G)R^{a}]2, —NR^{a}C(=G)OR^{a}, —C(=G)NR^{a}—NR^{a}₂, —C(=G)NR^{a}—NR^{a}[C(=G)R^{a}], —NR^{a}—C(=G)NR^{a}₂, —NR^{a}—NR^{a}C(=G)R^{a}, —NR^{a}—N[C(=G)R^{a}]₂, —N[(C=G)R^{a}]-NR^{a}₂, —NR^{a}—NR^{a}[(C=G)GR^{a}], —NR^{a}[(C=G)NR^{a}₂, —NR^{a}[C═NR^{a}]R^{a}, —NR^{a}(C═NR^{a})NR^{a}₂, O—NR^{a}₂, —O—NR^{a}(C=G)R^{a}, —SO₂NR^{a}₂, —NR^{a}SO₂R^{a}, —S(═O)R^{a}, —S(═O)₂R^{a}, —SO₂OR^{a}, and —OSO₂R^{a}; wherein
G is oxygen or sulfur;
R^{a} is each independently halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₈-halocycloalkenyl, ORⁱ, SRⁱ, S(═O)Rⁱ, S(═O)₂Rⁱ, NRⁱR^{j}, —S(═O)₂NRⁱR, C(═O)Rⁱ, C(═O)ORⁱ, C(═O)NRⁱR^{j}, C(═NORⁱ)R^{j}, —NRⁱC(=G)R^{j}, —N[C(=G)Rⁱ]2, —NRⁱC(=G)OR^{j}, —C(=G)NRⁱNR^{j}₂, —SO2NRⁱR^{j}, —NRⁱSO₂R^{j}, SiRⁱ_{y}R^{j}_{3-y} (y is 0 to 3), or
phenyl or a 5- to 6-membered heteraromatic ring which may contain 1 to 4 heteroatoms selected from oxygen, nitrogen and sulfur,
wherein the carbon atoms in phenyl or in the heteroaromatic ring may be substituted with 1 to 5 halogens;
Rⁱ, R^{j} are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₆-halocycloalkenyl, C₁-C₆-alkoxy, C₂-C₆-alkenyloxy, C₂-C₆-alkynyloxy, C₁-C₆-haloalkoxy, C₂-C₆-haloalkenyloxy;
R² is hydrogen, halogen, cyano, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₁-C₆-alkoxy, or C₁-C₆-haloalkoxy;
x is 2, 3, 4, 5, 6, or 7;
or the diastereomers, enantiomers, salts or N-oxides thereof, with the proviso that R¹ is not 5-chloro-2,2,-dimethyl-2,3-dihydrobenzo[b]furan-7-yl when R² is hydrogen and x is 3 or 4.

18. The compound of claim 17, wherein R³ is selected from the group consisting of halogen, C₁-C₆-alkyl, and C₁-C₆-haloalkyl.

19. The compound of claim 17, wherein R³ is selected from the group consisting of fluorine, chlorine, methyl and trifluoromethyl.

20. The compound of claim 17, wherein R² is halogen, methyl or trifluoromethyl.

21. The compound of claim 17, wherein R² is halogen.

22. The compound of claim 17, wherein R¹ is 2-pyridyl.

23. A process for the preparation of the compound of claim 17, comprising:
reacting a compound of formula (VII) with a compound of formula (VIII), optionally in the presence of a base to form a compound of formula (VI), 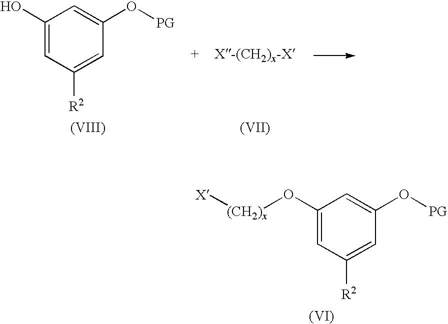
reacting a compound of formula (VI) with a compound of formula (V) in the presence of a base to form a compound of formula (IV) 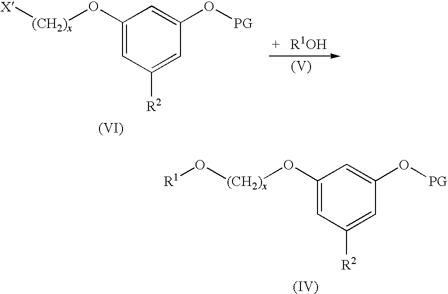
cleaving the protection group PG from the compound of formula (IV) to give a compound of formula II, and
reacting the compound of formula (II) with a compound of formula (III) in the presence of a base to give compounds I, 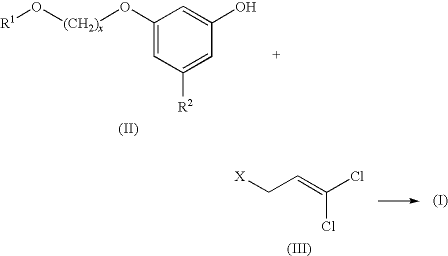
wherein the substituents R¹, R² and x in the above compounds have the meaning as defined in claim 17 for the compound of formula (I), PG is a protection group, and X, X′ and X″ are suitable leaving groups.

24. An intermediate compound of formula (IV), (VI), (IX), or (X), 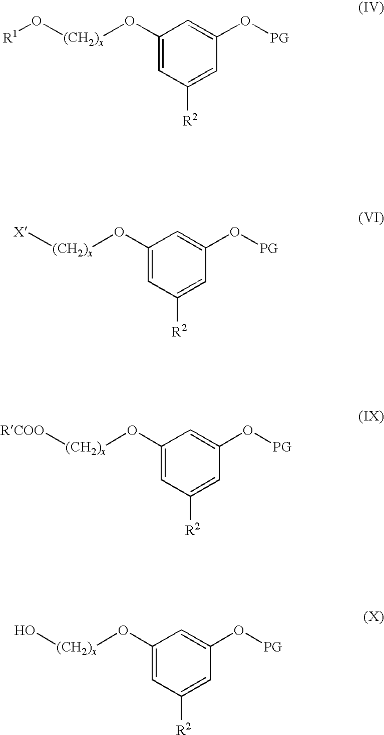
wherein
X′ is halogen, (C₁-C₄)alkylsulfonate, (C₄-C₆)haloalkylsulfonate or arylsulfonate;
PG is methyl, benzyl, benzyloxycarbonyl or methoxymethyl;
R′ is an aromatic or aliphatic residue;
R² is halogen; and
R¹ is pyridyl, which is substituted by any combination of 1 to 4 groups R³;
R³ is halogen, cyano, nitro, hydroxy, mercapto, amino, C₁-C₆-alkyl, C₂-C₆-alkenyl, C₂-C₆-alkynyl, C₃-C₆-cycloalkyl, C₃-C₆-cycloalkenyl, C₁-C₆-haloalkyl, C₂-C₆-haloalkenyl, C₂-C₆-haloalkynyl, C₃-C₆-halocycloalkyl, C₃-C₆-halocycloalkenyl, C₁-C₆-alkoxy, C₂-C₆-alkenyloxy, C₃-C₆-alkynyloxy, C₁-C₆-haloalkoxy, C₂-C₆-haloalkenyloxy, C₃-C₆-haloalkynyloxy, C₁-C₆-alkylthio, C₂-C₆-alkenylthio, C₃-C₆-alkynylthio, C₁-C₆-haloalkylthio, C₂-C₆-haloalkenylthio, C₃-C₆-haloalkynylthio C₁-C₆-alkylamino, C₂-C₆-alkenylamino, C₂-C₆-alkynylamino, di(C₁-C₆-alkyl)amino, di(C₂-C₆-alkenyl)amino, di(C₂-C₆-alkynyl)amino, C₁-C₆-alkyl-C₂-C₆-alkenylamino, C₁-C₆-alkyl-C₂-C₆-alkynylamino, C₁-C₆-alkenyl-C₂-C₆-alkynylamino, tri(C₁-C₁₀)alkylsilyl, or
phenyl or a 5- to 7-membered saturated or partially unsaturated heterocyclic ring which may contain 1 to 3 heteroatoms selected from the group consisting of oxygen, sulfur and nitrogen or
a 5- or 6-membered heteraromatic ring system which may contain 1 to 4 heteroatoms selected from the group consisting of oxygen, nitrogen and sulfur,
wherein the phenyl and the heteroaromatic ring may be bonded via an oxygen or a sulfur atom,
wherein each R³ group is unsubstituted or substituted with one or more substituents selected from the group consisting of —R^{a}, —C(=G)R^{a}, —C(=G)OR^{a}, —C(=G)NR^{a}₂, —C(=G)[N═SR^{a}₂], —C(═NOR^{a})R^{a}, —C(═NOR^{a})NR^{a}₂, —C(═NNR^{a}₂)R^{a}, —OC(=G)-OC(=G)OR^{a}, N═SR^{a}₂, —NR^{a}C(=G)R^{a}, —N[C(=G)R^{a}]2, —NR^{a}C(=G)OR^{a}, —C(=G)NR^{a}—NR^{a}₂, —C(=G)NR^{a}—NR^{a}[C(=G)R^{a}], —NR^{a}—C(=G)NR^{a}₂, —NR^{a}—NR^{a}C(=G)R^{a}, —NR^{a}—N[C(=G)R^{a}]₂, —N[(C=G)R^{a}]—NR^{a}₂, —NR^{a}—NR^{a}[(C=G)GR^{a}], —NR^{a}[(C=G)NR^{a}₂, —NR^{a}[C═NR^{a}]R^{a}, —NR^{a}(C═NR^{a})NR^{a}₂, —O—NR^{a}₂, —O—NR^{a}(C=G)R^{a}, —SO₂NR^{a}₂, —NR^{a}SO₂R^{a}, —S(═O)R^{a}, —S(═O)₂R^{a}, —SO₂OR^{a}, and —OSO₂R^{a}; wherein
G is oxygen or sulfur;
R^{a} is each independently halogen, cyano, nitro, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₈-halocycloalkenyl, ORⁱ, SRⁱ, S(═O)Rⁱ, S(═O)₂Rⁱ, NRⁱR^{j}, —S(═O)₂NRⁱR, C(═O)Rⁱ, C(═O)ORⁱ, C(═O)NRⁱR^{j}, C(═NORⁱ)R^{j}, —NRⁱC(=G)R^{j}, —N[C(=G)Rⁱ]2, —NRⁱC(=G)OR^{j}, —C(=G)NRⁱ—NR^{j}₂, —SO2NRⁱR^{j}, —NRⁱSO₂R^{j}, or SiRⁱ_{y}R^{j}_{3-y}; wherein y is an integer of 0 to 3; or
phenyl or a 5- to 6-membered heteraromatic ring which may contain 1 to 4 heteroatoms selected from oxygen, nitrogen and sulfur, wherein the carbon atoms in phenyl or in the heteroaromatic ring may be substituted with 1 to 5 halogens;
Rⁱ, R^{j} are each independently hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₆-cycloalkyl, C₃-C₈-halocycloalkyl, C₃-C₆-cycloalkenyl, C₃-C₆-halocycloalkenyl, C₁-C₆-alkoxy, C₂-C₆-alkenyloxy, C₂-C₆-alkynyloxy, C₁-C₆-haloalkoxy, C₂-C₆-haloalkenyloxy; and
x is 2, 3, 4, 5, 6, or 7.

25. A method for controlling insects, acarids or nematodes comprising contacting the insect, acarid or nematode or their food supply, habitat, breeding ground or their locus with a pesticidally effective amount of a composition comprising a compound of claim 17.

26. A method of protecting growing plants from attack or infestation by insects, acarids or nematodes comprising applying to the foliage of the plants, or to the soil or water in which they are growing, a pesticidally effective amount of a composition comprising a compound of claim 17.

27. The method of claim 25, wherein said compound is applied in an amount of from 5 g/ha to 2000 g/ha.

28. A method for treating, controlling, preventing or protecting an animal against infestation or infection by parasites which comprises orally, topically or parenterally administering or applying to the animal a parasiticidally effective amount of a composition comprising a compound of claim 17 or its veterinarily acceptable salt.

29. A composition comprising a pesticidally or parasiticidally active amount of a compound of claim 17 and an agronomically or veterinarily acceptable carrier.

30. A process for the preparation of a composition comprising a parasiticidally active amount of a compound of claim 17 and a veterinarily acceptable carrier said process comprises combining a parasiticidally effective amount of said compound or its veterinarily acceptable salt and a veterinarily acceptable carrier.
